(19) United States
(12) Patent Application Publication
(10) Pub. No.: US 2006/0247216 A1
(43) Pub. Date: Nov. 2, 2006

(54) Steroid compounds comprising superoxide dismutase mimic groups and nitric oxide donor groups, and their use in the preparation of medicaments

(76) Inventor: Abdullah I. Haj-Yehia, Neve Shalom (IL)

Correspondence Address: MORRISON & FOERSTER LLP, 755 PAGE MILL RD, PALO ALTO, CA, 94304-1018, US

(21) Appl. No.: 10/532,390

(22) PCT Filed: Oct. 24, 2003
(86) PCT No.: PCT/IL03/00878

§ 371 Date: Apr. 27, 2006

Related U.S. Application Data
(60) Provisional application No. 60/421,272, filed on Oct. 25, 2002.

(51) Int. Cl. A61K 31/655; A61K 31/58; A61K 31/56

(52) U.S. Cl. 514/149; 514/169; 514/176

(57) ABSTRACT

The present invention relates to multifunctional steroid compounds combining a steroid component with SOD mimic component and optionally also with NO donor component, and to their use in treating and preventing disorders associated with oxidative stress and free radical injury, or disorders in which treatment with steroids is indicated, whereas such combination increases the efficacy of treatment and reduces side effects associated with steroid treatment. The invention further relates to methods and devices for administering the compounds.

### FIELD OF THE INVENTION

The present invention relates to multifunctional steroid compounds that are capable of acting both as nitric oxide donors and as scavengers of reactive oxygen species such as superoxide, and which are useful in the treatment of conditions the pathogenesis of which involves oxidative stress and free radical injury (e.g., respiratory, inflammatory, and autoimmune disorders).

### BACKGROUND OF THE INVENTION

The relationship between reactive oxygen species (ROS) and nitric oxide (NO) plays a detrimental role in the modulation of many biological processes including aging, atherosclerosis, hypertension, diabetes mellitus, degenerative disorders, carcinogenesis, ischemia-reperfusion tissue injury, and acute and chronic inflammatory disorders. This is especially true in the case of inflammatory disorders of the respiratory system where oxidative stress exerted by ROS has been shown to significantly participate in the pathogenesis of, for example, adult respiratory distress syndrome (ARDS), emphysema, asthma, bronchopulmonary dysplasia, chronic obstructive pulmonary disease, and interstitial pulmonary fibrosis (Roche et al. (1989) Lancet 520-524; Soloperto et al. (1991) Am. J. Physiol. 260:L530-538; Montefort et al. (1992) Clin. Exp. Allergy 22:511-520).

NO (nitric oxide) is formed from the amino acid L-arginine by several forms of NO synthases, and plays a role in a number of physiological functions, including the relaxation of airway smooth muscle. NO formed in endothelial cells in response to chemical agonists and to physical stimuli plays a key role in regulation of vascular tone, platelet aggregation and adhesion, as well as modulating smooth muscle proliferation (Haj-Yehia et al. (2000) Drug Development Res. 50:528-536). NO overproduction has also been associated with numerous disease states (WO 99/66918). The production of NO is generally increased during inflammatory diseases such as rheumatoid arthritis, atherosclerosis, multiple sclerosis and asthma (Nathan (1992) FASEB J. 6:3051-3064; Gatson et al. (1994) Am. J. Respir. Crit. Care Med. 149:538-551; White et al. (1994) Proc. Natl. Acad. Sci. U.S.A. 91:1044-1048; Dweik et al. (1998) J. Clin. Invest. 101:660-666). These disorders are often referred to as oxidative stress-mediated diseases, where even higher increases in the production of superoxide and other ROS accompany the elevated production of NO.

The eventual fate of NO is oxidation to nitrite (NO₂⁻) and nitrate (NO₃⁻), which are both end-products of NO metabolism under normal conditions. However, under oxidative stress conditions, besides the depletion of the natural antioxidant capacity, the major metabolic pathway of NO involves reaction with superoxide, resulting in the formation of a highly potent ROS, peroxynitrite. Peroxynitrite is an extremely hazardous ROS capable of interrupting many physiological functions (Radi et al. (1991) J. Biol. Chem. 266:4244-4250; Beckman et al. (1990) Proc. Natl. Acad. Sci. U.S.A. 87:1620-1624; Beckman et al. (1993) Biochem. Soc. Trans. 21:330-334). NO levels have been shown to be increased in the asthmatic airways (Kaminsky et al. (1999) J. Allergy Clin. Immunol 104(4)I:747-754). The role of NO in the respiratory system has been studied (Tamaoki et al. (1995) Am. J. Physiol. 268(6)I:C1342-C1346). NO has also been used in the treatment of asthmatics, though such treatments demonstrated a great deal of inter- and intra-individual variability (WO 01/32202).

Publications disclosing nitric oxide donor compounds or compounds which promote the synthesis of nitric oxide include WO 98/42661, WO 99/37616, WO 00/31060, WO 97/34871, WO 00/35434, WO 99/62509, WO 97/25984, WO 00/67754, WO 9961018, WO 99/61430, WO 97/31654, WO 96/32946, WO 00/53191, WO 00/49993, WO 00/61604, U.S. Pat. Nos. 6,248,895 and 6,232,331 and Wolf et al. (1998) J. Neurosurg. 89:279-288. Publications disclosing nitric oxide scavenger compounds include WO 98/55453, U.S. Pat. Nos. 6,369,071 and 6,455,542.

The endothelium, in addition to producing NO, also produces superoxide (SO) anion and other reactive oxygen species (ROS) under physiological conditions. Despite SO being a reducing agent that is itself incapable of initiating oxidative reactions, SO is considered the most important source of oxidative stress. Compounds for the removal of SO are described in the art, including WO 96/39409, U.K. Pat. App. No. 2349385A, Krishna et al., (1998) J. Med. Chem, 41, 3477-3492. Publications disclosing additional bioantioxidants include Tat'yanenko et al., (1996) Pharm. Chem. J. 30(6), 361-36.

Many disease states, including diabetes mellitus and various cardiovascular diseases, are associated with oxidative stress and endothelial dysfunction. Nitroglycerin (GTN) has been used for the treatment of various types of myocardial ischemia Because of its pathogenic nature (chronicity with acute exacerbation), prophylactic and acute treatments are necessary to prevent complications with potentially fatal outcomes (>25% death for acute MI). However, the phenomenon of tolerance to the anti-anginal effects of GTN and to all other existing organic nitrates is of a special clinical significance. In particular, early development of tolerance to the drug is by far the most serious drawback of nitrate therapy.

A number of respiratory disorders have been recognized. Many of which have overlapping and interacting etiologies. The majority of these disorders are characterized by acute pulmonary vasoconstriction or bronchoconstriction. Inflammation and edema are also often associated with respiratory disorders such as asthma, respiratory distress syndrome (child or adult), bronchitis, pneumonia and others.

Various compounds and treatments for respiratory disorders are disclosed in the art, for example, in U.S. Pat. Nos. 6,299,863, 6,124,319, 6,197,762, 6,254,882, 6,083,993, 5,824,669, 5,821,259, RE 37,116E, WO 97/34871, WO 01/32202, WO 99/40787, WO 95/30641 and Australian Patent No. 733202.

Much progress has been made in our understanding of the role of the antioxidant enzymes, especially those involved in neutralizing superoxide (i.e., superoxide dismutase, SOD), in mediating the tissue resistance against oxidative stress and free radical injury.

Most current therapies for asthma aim either to affect the immune system (e.g. steroids) alone and/or to augment the physiological response of the lung to overcome the bronchoconstriction accompanying the disease (e.g., bronchodilators). However, none of these therapeutic modalities has been shown to adequately affect the natural course of the disease or its outcome as evident by the still high incidence of morbidity and mortality associated with asthma (Juniper et al. (1995) Am. J. Respir. Crit. Care Med. 151:66-70). This is conceivable since none of the current therapies address the multifactorial nature of the disease. In essence, however, many oxidative stress-mediated diseases like, for example, asthma, can be described as a disorder initiated by a yet unexplained hypersensitivity response of the trachepobronchial tree to allergens (allergic) that initiates an activation of the immune system (immunologic) to produce local inflammation (inflammatory) that results in bronchoconstriction of the involved tissue. As explained above, this simplified sequence of events leading to symptoms is accompanied by a significant increase of ROS production (oxidative stress). It has been recently reported that steroid therapy alone can lead to an increased production of superoxide, which in turn causes airway damage and hastens the progression of respiratory diseases. For a candidate drug to be effective both for prevention and acute treatment of such a disease, it has to adequately address each event of the sequence.

The development of inhaled steroids which are effective without significant systemic effects has been a major advance in the treatment of asthma. As many as 80% of patients depend on systemic steroids which may be managed with inhaled steroids. There is no clear advantage for any currently available inhaled steroids. Aggressive dosing of inhaled steroids is being advocated as a means of decreasing systemic steroid doses, although acute adrenal insufficiency may result, and when used for prolonged periods, osteoporosis may be of concern (Wong et al. (1992) BMJ 304:1415-22). Inhaled steroids are indicated in any patient requiring continuous P2-agonists or even as first line therapy according to some authors (Lam et al. (1990) Chest 98:44-52; Haahtela et al. (1991) NEJM 325:388-92).

Various compounds and treatments for cardiovascular disorders are disclosed in the art, for example, in U.S. Pat. Nos. 6,444,702, 6,417,207, 6,255,296, 6,051,571, 6,440,961, 6,429,219, 6,423,724, 6,248,895, 6,218,417, 5,780,495, 5,700,947, 5,621,000, 6,040,341, 5,861,426, and 6,242,432, and WO 00/49993. Publications disclosing compounds for the treatment of male impotence include U.S. Pat. No. 6,211,233.

Similarly, compounds and treatments for migraines are disclosed in the art, for example, U.S. Pat. Nos. 6,458,840, 6,458,830, 6,444,702, 6,376,550, 6,414,505, 6,403,627, 6,355,689, 6,331,553, 6,265,441, 6,423,724, and 6,455,549.

Various compounds and treatments for sinus tachycardia are disclosed in the art, for example, U.S. Pat. No. 6,100,297.

Compounds and treatments for hypertension are disclosed in the art, for example, U.S. Pat. Nos. 6,440,961, 6,429,219, 6,423,724, 6,214,817, and 6,455,542.

Various compounds and treatments for the symptoms of hyperthyroidism are also disclosed in the art, for example, U.S. Pat. Nos. 6,110,959, 6,121,309, and 6,437,165.

There is a need for improved drugs for the treatment of respiratory disorders such as asthma, and other disorders in which treatment with steroids is indicated.

### SUMMARY OF THE INVENTION

Multifunctional steroid compounds are provided, and compositions comprising multifunctional steroid compounds, for the prophylaxis and/or treatment of conditions the pathogenesis of which involves oxidative stress and free radical injury, disorders in which treatment with steroids or their analogs is indicated, or disorders in which treatment with a smooth muscle relaxant is indicated, (e.g., respiratory, inflammatory, and autoimmune disorders). Also provided are methods of using the multifunctional steroid compounds and multifunctional steroid compositions described herein for the prophylaxis and/or treatment of respiratory disorders, respiratory distress or related disorders or symptoms thereof, including but not limited to COPD, asthma, respiratory distress syndrome (child or adult), pneumonia, chronic bronchitis or emphysema. Further, the multifunctional steroid compounds and compositions described herein may be used in the prophylaxis and/or treatment of other disorders in which treatment with steroids is indicated (e.g., allergic conditions, arthritis, skin conditions, fertility conditions, reproductive disorders, inflammatory bowel diseases, neurodegenerative disorders, etc.).

The multifunctional steroid compounds described herein are characterized in comprising at least one superoxide dismutase (SOD) mimic component and a steroid component, and optionally at least one NO donor component. The compounds may include at least one NO donor component and at least one SOD mimic component linked to a steroid component. In other embodiments, functional steroid compounds are provided that include at least one SOD mimic component linked to a steroid component, which can be made and used as described herein for multifunctional steroid compounds.

This invention relates to a multifunctional steroid compound of formula 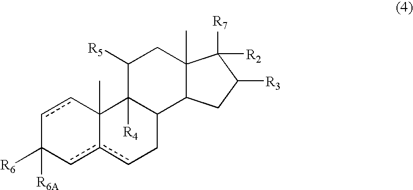
optical isomers thereof, salts thereof, and solvates thereof;
wherein [symbol] is a single or double bond, with the proviso that two double bonds are not adjacent;
R² is —H, —ONO, —ONO₂, —SNO, —OH, —CH₃, —NONOate, —OC(O)R⁸ wherein R⁸ is C₁-C₅ alkyl or 5- or 6-member heteroaryl, or or R² and R⁷ together form a substituted N-oxide free radical;
R³ is —H, —OH, or —H₃, or R² and R³ together form a heterocyclic ring;
R⁴ is —H or halogen;
R⁵ is —H, ═O, —ONO, —ONO₂, —SNO, —NONOate or a substituted N-oxide free radical;
R⁶ is ═O, —ONO, —ONO₂, —SNO, —NONOate;
R^{6A}, if present, is —H, or R⁶ and R^{6A} together form a substituted N-oxide free radical;
R⁷ is —H, —ONO, —ONO₂, —SNO, —NONOate, or a substituted N-oxide free radical wherein the nitrogen of the N-oxide group in the substituted N-oxide free radical is within a 5- or 6-member ring, which ring is optionally substituted by —OCOCH₂-PEG wherein said PEG may by optionally coupled to another steroid compound, and which ring is further optionally substituted by or one or more independently selected C₁-C₅ alkyl groups which may be further independently substituted by a group selected from an NO donor component, —SR¹¹, -halogen, and —OC(O)R¹³ wherein R¹¹ is C₁-C₅ alkyl and wherein R¹³ is C₁-C₅ alkyl or 5- or 6-member heteroaryl, or R² and R⁷ together form a substituted N-oxide free radical; and wherein NO donor is a group comprising one of —ONO₂, —ONO, —SNO, and —NONOate, and wherein the nitrogen of the N-oxide group in the substituted N-oxide free radical is within a 5- or 6-member ring substituted by one or more independently selected C₁-C₅ alkyl groups which may be may be further independently substituted by an NO donor component.

This invention also relates to a dimer steroid compound in which PEG links two, preferably identical, steroid structures, preferably selected from Ia to Id, IIa to IId, IIa to IIId, and IVa to IVd 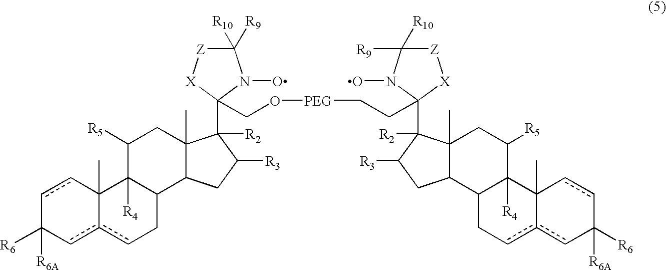
wherein the R², R³, R⁴, R⁵, R⁶, and R^{6A} are as defined above;
R⁹ and R¹⁰ are independently, linear or branched C₁-C₅ alkyl groups, or substituted linear or branched C₁-C₅ alkyl groups wherein the alkyl group is independently substituted by an NO donor or —OC(O)R¹⁴, wherein R¹⁴ is C₁-C₅ alkyl, or 5- or 6-member heteroaryl;
X is —CH—, —O— or —S—;
Z is —CH₂— or —CH₂—CH₂—;
and PEG is a polyethylene glycol of a molecular weight preferably from about 100 to about 4000.

The multifunctional steroid compounds include, but are not limited to the multifunctional steroid compounds of formulae I (Ia-Id), II (IIa-IId), III (IIIa-IIId), IV (IVa-IVd), V (Va-Vd), and VI (VIa-VId).

Accordingly, in certain embodiments, the multifunctional steroid compound includes a compound of formulae I, wherein

R² is —H, or —ONO₂;

R³ is —H, —OH, or —CH₃;

R⁴ is —H, —F or —Cl;

R⁵ is —H, ═O, or —ONO₂;

R⁶ is ═O, or —ONO₂ and R^{6A}, if present, is —H, or R⁶ and R^{6A} together form a substituted N-oxide free radical selected from the group consisting of substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical and substituted thiazinyloxy N-oxide free radical; and

R⁷ is H or —ONO₂ or a substituted N-oxide free radical selected from the group consisting of substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical and substituted thiazinyloxy N-oxide free radical, or

R² and R⁷ together form a substituted N-oxide free radical,

wherein at least one of R², R⁵, R⁶, or R⁷ comprises an NO donor; and

wherein at least one of R⁶/R⁶A or R⁷ comprises a substituted N-oxide free radical.

In other embodiments, the multifunctional steroid compounds include a compound according to formulae III, wherein
R¹ is —H, —SNO or —NO₂;
R² is —H, or —ONO₂;
R³ is —H, H, or —CH₃;
R⁴ is —H, —F or —Cl;
R⁵ is —H, ═O, or —ONO₂;
R⁶ is ═O or —NO₂ and R^{6A}, if present, is —H, or R⁶ and R^{6A} together form a substituted N-oxide free radical selected from the group consisting of substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical and substituted thiazinyloxy N-oxide free radical;
R⁹ and R¹⁰ are independently C₁-C₂ alkyl;
X is —O— or —CH₂—; and
Z is —CH₂—;
wherein at least one of R¹, R², R⁵, or R⁶ comprises at least one NO donor.

In particular embodiments of the multifunctional steroid compounds of formulae I or III, R⁶ and R^{6A} together from a N-oxide free radical selected from the group consisting of substituted 3-oxazolidinyloxy free radicals.

In some embodiments of the multifunctional steroid compounds of formulae I or III, R⁶═O.

In particular embodiments of the multifunctional steroid compounds of formulae I or III, the ratio of N-oxide free radical: NO donor group is 1:1 or 2:1.

In some embodiments of the multifunctional steroid compounds, the multifunctional steroid compounds may include compounds 1-23, as shown in FIGS. 1, 2, 3, 4 and 5.

In particular embodiments is provided a multifunctional steroid compound as described herein, including compounds according to formulae I (Ia-Id), II (IIa-IId), III (IIIa-IIId), IV (IVa-IVd), V (Va-Vd), or VI (VIa-VId), and a pharmaceutically acceptable excipient. In some embodiments, the compounds of formulae I (Ia-Id), II (IIa-IId), III (IIIa-IIId), IV (IVa-IVd), V (Va-Vd), or VI (VIa-VId) do not include an NO donor group.

This invention is directed to the use of compounds of formulae (4), (5), and I to VI in the preparation of a medicament for treating and preventing a disorder selected from the group consisting of asthma, chronic bronchitis, bronchiectasis, bronchospasms, emphysema, pneumonia, Chronic Obstructive Pulmonary Diseases (COPDs), bronchial hyperreactivity, respiratory distress syndrome or Chronic Obstructive Airway Disease (COADs), allergic conditions, arthritis, autoimmune hematologic disorders, systemic lupus erythematosus, systemic dermatomyositis, thrombocytopenia, psoriasis, contact dermatitis, atopic dermatitis, exfoliative dermatitis, acne, hirsutism, erythema nodosum, inflamed cysts, discoid lupus, bullous diseases, collagen vascular diseases, malignancies, neoplastic disease, trauma, shock, acute and chronic inflammatory conditions, sarcoidosis, Sweet's disease, graft-versus-host disease, multiple sclerosis, Alzheimer diseases, Parkinson's diseases, amyotrophic lateral sclerosis, convulsive disorders, AIDS-dementia, disorders related to learning, disorders related to olfaction, disorders related to nociception, cerebral edema, migraine, ophthalmic disorders, chronic adrenal insufficiency, congenital adrenal hyperplasia, gastrointestinal diseases, hepatic diseases, inflammatory bowel disease, Crohn's disease, ulcerative colitis, renal disease, gastric secretory and peristaltic functions, drug and disease-induced neuropathies and nephropathies, pathological uterine contractions, sinus tachycardia, ischaemic heart disease, angina pectoris, myocardial infarction, congestive heart failure, atherosclerosis, rheumatic disorders, hypertension, arrhythmia, hyperthyroidism, cellular defense impairment, hypercholestemia, Reaven's Syndrome, vasculitis, arteritis, endothelial dysfunction-induced diseases, diabetes mellitus, insulin-resistance and glucose intolerance in diabetes, ischemia-reperfusion tissue injury, chemotaxis and phagocytic impairment in immunological disorders, aging-mediated changes, cerebrovascular diseases, thyrotoxicosis, aggregation disorders, fertility conditions and reproductive disorders, menopause, ovarian dysfunction, testicular dysfunction, and penile erection.

Certain aspects of the multifunctional steroid compounds described herein include an inhalation device comprising a multifunctional steroid compound, an inhaler and pharmaceutically acceptable carrier or aerosolizer. In some embodiments, the multifunctional steroid compound is a compound according to formulae I (Ia-Id), II (IIa-IId), III (IIIa-IIId), IV (IVa-IVd), V (Va-Vd), or VI (VIa-VId).

Another aspect of the multifunctional steroid compound comprises a kit for the treatment of a respiratory condition in an individual in need thereof, comprising the inhalation device as described above, packaging and instructions for use.

In another aspect of the multifunctional steroid compounds includes a method for treating a respiratory condition in an individual in need thereof, comprising administering an effective amount of a multifunctional steroid compound as described herein to said individual. In some embodiments, the multifunctional steroid compound is a compound according to formulae I (Ia-Id), II (IIa-IId), III (IIIa-IIId), IV (IVa-IVd), V (Va-Vd), or VI (VIa-VId). In certain embodiments, compounds according to formulae I (Ia-Id), II (IIa-IId), III (IIIa-IIId), IV (IVa-IVd), V (Va-Vd), or VI (VIa-VId), are provided where the compound includes an SOD mimic component but does not include an NO donor group.

In particular embodiments, the multifunctional steroid compound is administered orally.

In some embodiments, the compound is administered by inhalation.

In particular embodiments, the respiratory condition is asthma, chronic obstructive pulmonary disease, bronchial hyperreactivity, adult respiratory distress syndrome, emphysema, bronchopulmonary dysplasia, or interstitial pulmonary fibrosis.

Another embodiment includes a method of treating a condition in an individual in need thereof comprising administering an effective amount of a compound of a multifunctional steroid compound to said individual, wherein the condition is selected from the group consisting of allergic conditions, skin conditions, fertility conditions, reproductive disorders, inflammatory bowel diseases and multiple sclerosis. In certain embodiments, the multifuctional steroid compound is a compound according to formulae I (Ia-Id), II (IIa-IId), III (IIIa-IIId), IV (IVa-IVd), V (Va-Vd), or VI (VIa-VId).

In some of the embodiments of the methods described herein, the compound is administered orally. In others, the compound is administered topically.

In some embodiments of the methods as described herein, the condition is multiple sclerosis.

In other embodiments, the condition is a skin condition such as psoriasis, atopic dermatitis, or contact dermatitis. The compound may be administered topically.

In another embodiment is a multifunctional steroid compound comprising a steroid component, at least one superoxide dismutase (SOD) mimic component and at least one nitric oxide donor component.

In some embodiments, the steroid component is a steroid component of beclomethasone, budesonide, fluticasone, mometasone, dexamethasone, clobetasone, or betamethasone.

In some embodiments, the steroid component is a steroid component of beclomethasone, budesonide, prednisone, prednisolone, or fluticasone.

In certain embodiments, the steroid component is a steroid component of mometasone, dexamethasone, clobetasone, prednisone, prednisolone, or betamethasone.

In some embodiments, the steroid component is a steroid component of prednisone, prednisolone, or dexamethasone.

In particular embodiments, the compound comprises two SOD mimic components.

In some embodiments, the at least one nitric oxide donor component is independently —ONO, —ONO₂, —SNO or —NONOate.

In another embodiments, the at least one nitric oxide donor component is independently —ONO₂, or —SNO. In others, —ONO₂.

In certain embodiments, the at least one SOD mimic component is a substituted N-oxide free radical in which the nitrogen of the N-oxide group of the substituted N-oxide free radical is within a 5- or 6-member ring.

In other embodiments, the at least one substituted N-oxide free radical is independently selected from the group consisting of pyrrolidinyloxy free radicals, piperidinyloxy free radicals, oxazolidinyloxy free radicals, oxazinyloxy free radicals, thiazolidinyloxy free radicals and thiazinyloxy free radicals.

In certain embodiments, the substituted N-oxide free radical is a substituted 3-oxazolidinyloxy free radical.

In some embodiments, the compound comprises at least two nitric oxide donor components.

In some embodiments the compound comprises two nitric oxide donor components and two SOD mimic components.

In particular embodiments, the ratio of NO donor component:SOD mimic component of 1:1, 2:1 or 1:2.

In some embodiments, is a composition is provided comprising a multifunctional steroid compound and a pharmaceutically acceptable excipient, in pharmaceutically acceptable form. In certain embodiments, the multifunctional steroid compound is a compound according to formulae I (Ia-Id), II (IIa-IId), III (IIIa-IIId), IV (IVa-IVd), V (Va-Vd), or VI (VIa-VId).

In certain methods of the methods of described herein, the multifunctional steroid compound is administered once or twice daily.

In some methods of the methods of described herein, the condition is multiple sclerosis or inflammatory bowel disease. In certain embodiments, the multifunctional steroid compound is administered orally or intravenously.

In some methods of the methods of described herein, the condition is an allergic condition, such as rheumatoid arthritis, osteoarthritis, allergic rhinitus, asthma, or atopic dermatitis.

### BRIEF DESCRIPTION OF THE DRAWINGS

FIG. 1 shows exemplary compounds 1-8.

FIG. 2 shows exemplary compounds 9-16.

FIG. 3 shows exemplary compounds 17-18.

FIG. 4 shows exemplary compounds 19-21.

FIG. 5 shows exemplary compounds 22-23.

FIG. 6 shows a scheme for the synthesis of exemplary dimer compound D.

FIG. 7 shows a scheme for the synthesis of exemplary dimer compound H.

### DETAILED DESCRIPTION OF THE INVENTION

Provided are multifunctional steroid compounds for the treatment of respiratory and other disorders treated by steroid administration (e.g. allergic conditions, autoimmune conditions, skin conditions (including psoriasis, atopic dermatitis and contact dermatitis), multiple sclerosis , inflammatory bowel disease, fertility conditions (e.g., testicular dysfunction, ovarian dysfunction, menopause), etc.). The multifunctional steroid compound includes a steroid component, a superoxide dismutase (SOD) mimic component and a nitric oxide donor component. Thus, in one embodiment, a steroid is provided in modified form and includes a superoxide dismutase (SOD) mimic component and a nitric oxide donor component capable of releasing NO in a charged or neutral form. The steroid component may be linked to at least one superoxide dismutase (SOD) mimic component and at least one nitric oxide donor component. Exemplary steroids include, but are not limited to androsterone, epiandrosterone, progesterone, testosterone, pregnenolone, cortisone, hydrocortisone, dexamethasone, prednisone, prednisolone, beclomethasone and budesonide. In certain conditions the steroids from which the steroid component is selected is a hormonal steroid (e.g., estrogen, progesterone, testosterone and designed analogues thereof (e.g. estradiol)). Where the condition intended to be treated is a respiratory disorder or acute allergic reaction, the steroid component may be a steroid component of beclomethasone, budesonide, prednisone, prednisolone, or fluticasone. Where the condition intended to be treated is a skin disorder (e.g., psoriasis) or inflammatory disorder (e.g., rheumatoid arthritis, multiple sclerosis, inflammatory bowel disease (e.g., ulcerative colitis), vasculitis (e.g., Takayasu's and Kawasaki's diseases, etc.)), the steroid component may be a steroid component of mometasone, dexamethasone, clobetasone, prednisone, prednisolone, or betamethasone. Where the condition intended to be treated is a disorder associated with increased intracranial pressure or brain edema (e.g., after injury or secondary to malignancy), the steroid component may be a steroid component of prednisone, prednisolone, or dexamethasone. The nitric oxide donors include —ONO, ONO₂, —SNO and —NONOate. The SOD mimic component is, for example, a substituted N-oxide free radical, such as, for example a substituted pyrrolidine N-oxide free radical, substituted piperidine N-oxide free radical or substituted oxazolidine N-oxide free radical.

In one embodiment, the invention relates to nitrosated or nitrosylated steroid-derived SOD mimic compounds which can optionally be substituted with at least one —NO, —SNO, or —ONO₂ moiety, or substituted with a group that donates, transfers, or releases nitric oxide in either a neutral or a charged form.

The multifunctional steroid compounds described herein offer a new strategy for the treatment of asthma and other inflammatory conditions that can affect not only the clinical symptoms of the disease, but also its pathogenesis, natural course and outcome.

The beneficial therapeutic effects of the multifunctional steroid compounds described herein, without being limited to any theory, may be attributed to their simultaneous multi-mechanistic actions, possibly comprising synergism, as steroids (immunosuppressant, anti-inflammatory, anti-allergic), SOD-mimics (antioxidant and anti-inflammatory that provide additional cellular protection), and as NO donors (antioxidant, anti-proliferative, cellular protectant with potent smooth muscle relaxing properties). These properties are useful for adequate prevention and treatment of acute episodes of inflammatory disorders involving allergy, immune stimulation and proliferation, depletion of natural antioxidants, and bronchoconstriction, as is the case in, for example, asthma.

The multifunctional steroid compounds, and compositions comprising the multifunctional steroid compounds, may be used in methods of treating respiratory disorders including asthma, bronchitis, emphysema, bronchospasms, pneumonia, bronchial hyperreactivity, respiratory distress syndrome and other ailments in patients with oxidative stress-mediated conditions. The multifunctional steroid compounds, compositions comprising the multifunctional steroid compounds and methods described herein are also directed to avoiding adverse effects, development of tolerance (e.g. desensitization) or hypersensitivity on repeated administration. The multifunctional steroid compounds and compositions comprising the multifunctional steroid compounds as described herein may also be used in the manufacture of medicaments for the treatment of respiratory and other conditions in which treatment with steroids is indicated.

The multifunctional steroid compounds, and compositions comprising the multifunctional steroid compounds, may be used in methods of treating conditions where treatment with steroids (including designed analogues) is indicated. Such conditions include, but are not limited to: respiratory disorders (e.g., asthma, chronic bronchitis, bronchiectasis, bronchospasms, emphysema, Chronic Obstructive Pulmonary Diseases (COPDs), bronchial hyperreactivity, respiratory distress syndrome or Chronic Obstructive Airway Disease (COADs), the treatment of allergic conditions (e.g., rhinitis and sinusitis), arthritis (e.g. rheumatoid or osteo arthritis), autoimmune conditions (e.g. autoimmune destruction of erythrocytes, autoimmune hematologic disorders, systemic lupus erythematous, graft-vs.-host disease, etc.), cerebral edema, chronic adrenal insufficiency, congenital adrenal hyperplasia, gastrointestinal diseases, hepatic diseases, inflammatory bowel disease, malignancies, multiple sclerosis, neoplastic disease, ocular diseases, ophthalmic disorders, transplantation including bone marrow and organ transplantation, skin conditions (e.g. psoriasis, contact dermatitis, atopic dermatitis, exfoliative dermatitis, acne, hirsutism, erythema nodosum, inflamed cysts, discoid lupus, bullous diseases, collagen vascular diseases, sarcoidosis, Sweet's disease), renal disease, rheumatic disorders, sarcoidosis, systemic dermatomyositis, cancer, and thrombocytopenia.

The use of steroids for the treatment of the above-listed conditions are known to those of skill in the art (see, for example Goodman & Gillman, supra; Remington: The Science and practice of Pharmacy 20th Ed. (2000) Lippincott Williams and Wilkins, Ed. K. E. Hoover, Merck Index, Sanders et al. Am. J. Respir. Crit. Care. Med., (1995) 151: 1725-33) and the use of the multifunctional steroid compounds described herein in the treatment of these conditions has the benefit of increasing the efficacy of the treatment while decreasing the side effects associated with steroid treatment, and lowering toxicity.

The multifunctional steroid compounds of the present invention may be employed in the treatment of conditions associated with endothelial dysfunction or oxidative stress including diabetes mellitus, cardiovascular diseases (such as ischaemic heart disease, angina pectoris, myocardial infarction, congestive heart failure, atherosclerosis (e.g., ateriosclerosis), hypertension (e.g., pulmonary, systemic, ocular or pregnancy-induced) and arrhythmia), vasculitis, arteritis (e.g., temporal arteritis), respiratory disorders (e.g., asthma, chronic bronchitis, bronchiectasis, bronchospasms, emphysema, Chronic Obstructive Pulmonary Diseases (COPDs), bronchial hyperreactivity, respiratory distress syndrome or Chronic Obstructive Airway Disease (COADs)), trauma, shock (hypovolumic, neurogenic or septic), neurotoxicity, neurodegenerative and neurological disorders (including Alzheimer and Parkinson's diseases, amyotrophic lateral sclerosis, multiple sclerosis, convulsive (seizure) disorders, AIDS-dementia and disorders which involve processes of learning, olfaction, nociception and memory), disorders of gastric acid and other secretory and peristaltic functions of the alimentary system (including relaxation and peristalsis of the intestinal tract (including sphincters)), drug and disease-induced neuropathies and nephropathies, pathological (premature) and physiological uterine contractions, migraine, sinus tachycardia, the symptoms of hyperthyroidism, cellular defense impairment, acute and chronic inflammatory conditions, diabetes mellitus (including the complications thereof, e.g., hypercholestemia, hypertension, atherosclerosis or Reaven's Syndrome, otherwise known as Syndrome-X), endothelial dysfunction-induced diseases, insulin-resistance and glucose intolerance in diabetes, ischemia-reperfusion tissue injury, chemotaxis and phagocytic impairment in immunological disorders, aging and aging-mediated changes (e.g., premature balding, senescence-associated changes in skin and appearance), cerebrovascular diseases, thyrotoxicosis, aggregation disorders, fertility conditions and reproductive disorders (e.g., menopause, ovarian dysfunction, testicular dysfunction, penile erection and the treatment of male impotence). The compounds of the present invention can also be used in the treatment of allergic conditions, arthritis (e.g. rheumatoid or osteo arthritis), autoimmune conditions (e.g. autoimmune destruction of erythrocytes, autoimmune hematologic disorders, systemic lupus erythematosus, graft-vs.-host disease, etc.), cerebral/brain edema, increased intracranial pressure (e.g., as associated with injury or secondary to malignancy, etc.) chronic adrenal insufficiency, congenital adrenal hyperplasia, gastrointestinal diseases, hepatic diseases, inflammatory bowel diseases (e.g., Crohn's disease and ulcerative colitis), vasculitis (e.g., Takayasu's and Kawasaki's diseases, etc.), malignancies, multiple sclerosis, neoplastic disease, ocular diseases, ophthalmic disorders (e.g., cataracts, retinopathy, glaucoma, corneal disease, etc.), transplantation including bone marrow and organ transplantation, skin conditions (e.g. psoriasis, contact dermatitis, atopic dermatitis, exfoliative dermatitis, acne, hirsutism, erythema nodosum, inflamed cysts, discoid lupus, bullous diseases, collagen vascular diseases, sarcoidosis, Sweet's disease), renal disease, rheumatic disorders, sarcoidosis, systemic dermatomyositis, cancer, and thrombocytopenia.

The use of the multifunctional steroid compounds described herein may be of particular use in the treatment of allergic conditions, including skin conditions, for example, psoriasis, contact dermatitis, atopic dermatitis; multiple sclerosis; inflammatory bowel disease; neurodegenerative disorders (e.g. multiple sclerosis, etc.); fertility conditions and reproductive disorders, for example, menopause, ovarian dysfuntion, testicular dysfunction; inflammatory bowel diseases (e.g., Chron's disease or ulcerative colitis); and respiratory disorders, as, for example, asthma, COPD, ARDS, etc.

The multifunctional steroid compounds and compositions comprising the multifunctional steroid compounds as described herein may also be used in the manufacture of medicaments for the treatment of disorders in which treatment with steroids (including designed analogues) is indicated. These include where the treatment with hormonal steroids is indicated (e.g., ovarian dysfunction, testicular dysfunction, menopause).

The multifunctional steroid compounds, and compositions comprising the multifunctional steroid compounds, described herein not only provide a source of nitric oxide, which acts in the regulation of airway smooth muscle, but in acting as an antioxidant scavenger of superoxide anion and other reactive oxygen species give rise to both a direct benefit derived from removal of injurious superoxide anion and other reactive oxygen species and a benefit in protecting both ambient and endogenous and liberated exogenous NO from inactivation by superoxide anion and other reactive oxygen species, while the steroid component has a steroid function such as an anti-inflammatory and/or immunomodulating effect. See, for example: Hart Chest 15: 1407-1417 (1999); Dweski Thorax. 55 (Suppl 2): 551-553. (2000); Muntuschi et al. Am. J. Resp. Crit. Care Med. 160(1): 216-220 (1999); Benjamin et al. The Lancet 351: 1317-1319 (1998); Benjamin et al Am. J. Respir. Care Med. 149: 538-551 (1994); Hilliwell Oxford University Press Pp: 1-685. (1999); Chabot et al. Eur. Respir. J. 11: 745-757 (1998); Rahman Free Rad. Biol. Med. 21: 669-681 (1996); Kanazawa et al. Chest 100: 1319-22 (1991); Sanders et al. Am. J. Respir. Crit. Care Med. 151: 1725-33 (1995); Saleh et al. FASEB J. 12: 929-937 (1998).

As used herein, the term “multifunctional steroid compound” refers to a compound containing a steroid component and at least one SOD mimic component, and optionally at least one NO donor component. The components may be linked, for example directly, indirectly and/or via a sharing of atoms, as described herein. In one embodiment, a known steroid is chemically modified to form the multifunctional steroid compound. The use of the term “multifunctional steroid compound” is not intended to necessarily require that the compound was formed by chemical modification of a steroid, since the synthesis would not necessarily involve a starting material that was a steroid that is further modified, and other routes of synthesis are contemplated. Rather, a “multifunctional steroid compound” is meant to be a molecule that not only includes a steroid component with anti-inflammatory and/or immunomodulating activity or other steroid activity, but also the additional functionality of the NO and donor SOD mimic components. The steroid component is the component with the activity of a steroid, and may be the component that results after modification of a steroid to include the NO donor component and SOD mimic component. Thus, in one embodiment, multifunctional steroid compounds are provided that are a steroid in a modified form wherein they include an NO donor component and a SOD mimic component.

The multifunctional steroid compound may comprise at least one group that affords SOD-mimic activity and added anti-inflammatory action, and at least one —NO, —SNO, or —ONO₂ moiety that confers on the SOD-mimic steroid an additional relaxant effect with all other beneficial biological actions expected from anNOdonor.

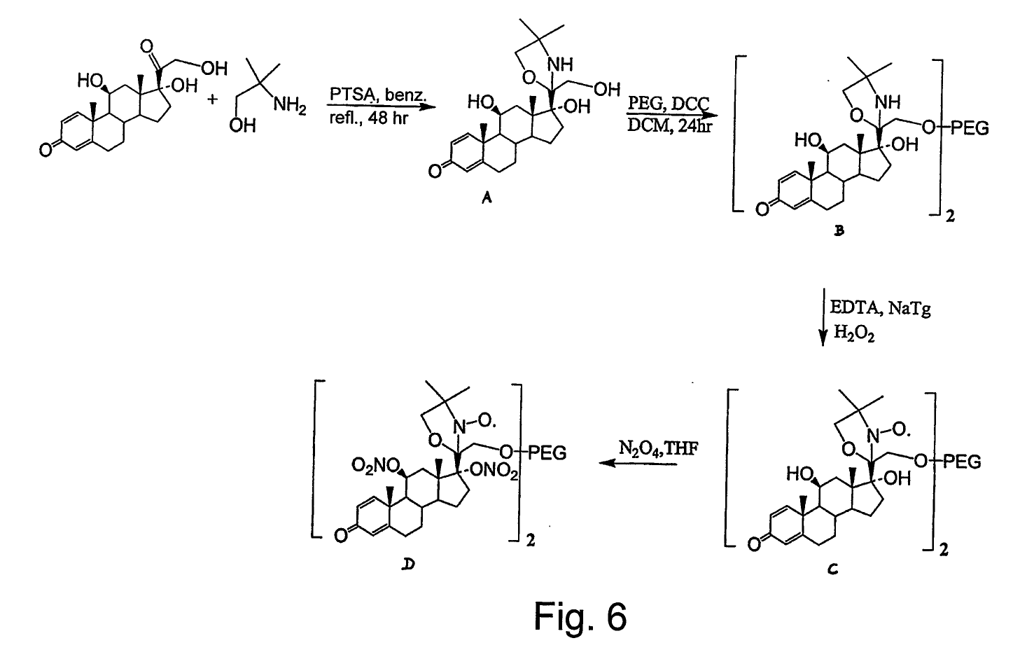

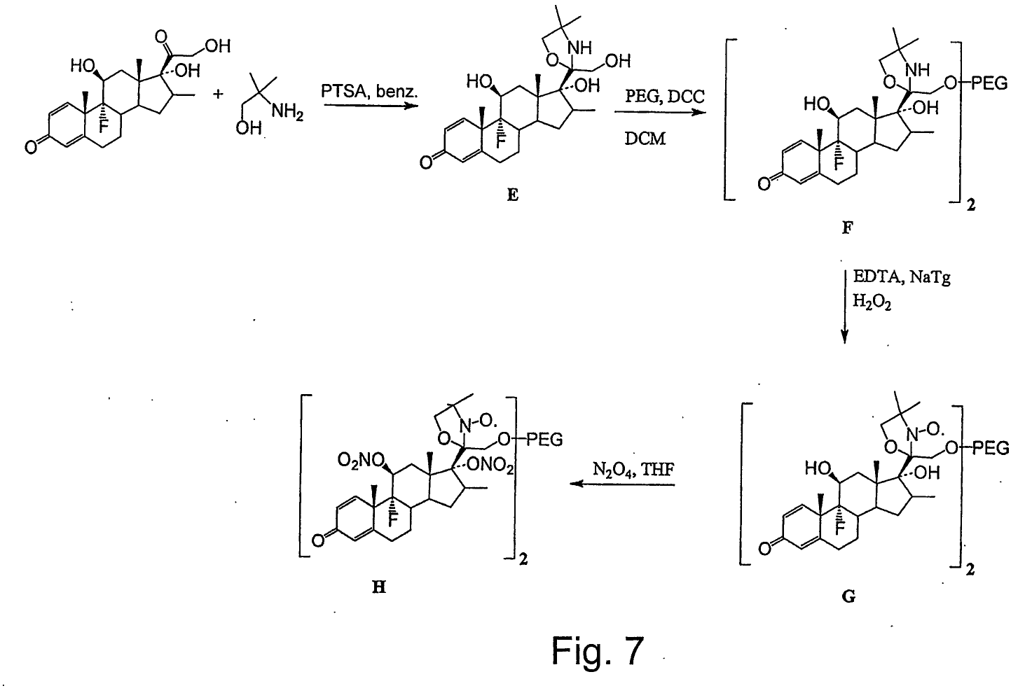

In another embodiment, a multimer is provided that includes a steroid component modified with a SOD mimic component, such as a substituted oxazoladinyl free radical connected, for example, via a hydroxyl group, to one end of a polyethylene glycol (PEG) or other spacer. The other terminus of the spacer, such as PEG, then may be covalently bonded to a second steroid component modified with a SOD-mimic component. The multimer optionally is further substituted with at least one —ONO, —SNO, or —ONO₂ moiety, or a moiety that donates, transfers, or releases nitric oxide in either a neutral or a charged form. Examples are shown in FIGS. 6 and 7.

A spacer like PEG is a well known cell permeable, non-toxic, non-mutagenic molecule that favorably affects the polarity of the final product allowing its easy introduction into a wide variety of pharmaceutical formulations.

Examples of contemplated steroids from which steroid components may be selected include, but are not limited to, androsterone, epiandrosterone, progesterone, testosterone, pregnenolone, cortisone, hydrocortisone, dexamethasone, prednisone, prednisolone, beclomethasone and budesonide. Examplary hormonal steroids from which steroid components may be selected include, estrogens (e.g., estradiol), progesterone, androgens (e.g., testosterone) and designed and natural analogues thereof.

NO Donors

Groups that can act as nitric oxide donors are capable of acting as a source of nitric oxide (NO). The nitric oxide donor component is, for example, an —ONO₂ (organic), —ONO (inorganic), —SNO, or —NONOate group. In particular embodiments the NO donor component is —ONO₂ or —SNO. The NO donor component, for example, donates, transfers, or releases nitric oxide in either a neutral or a charged form. The nitric oxide donor component may comprise any group capable of acting as a source of nitric oxide (NO) in a charged or uncharged form, including nitrosonium (NO⁺), nitroxyl (NO⁻) or nitric oxide (NO.).

In particular embodiments of the multifunctional steroid compounds, the compounds may comprise more than one NO donor component, for example, at least one, at least two, at least three or at least four NO donor components. The multifunctional steroid compound may include one or more of the same or different NO donor components.

Superoxide Dismutase Mimics

The multifunctional steroid compound may include an antioxidant that preferentially scavenges, or reacts with, superoxide, which is termed a “superoxide dismutase mimic” component (“SOD-mimic”) or “superoxide dismutase mimetic” component (“SOD-mimetic). The reactive oxygen species superoxide (O₂⁻) is considered biologically undesirable, while nitric oxide, may be biologically beneficial. Thus, the SOD mimic component preferably does not react with, or scavenge, nitric oxide. In some embodiments, the SOD mimic component is a substituted N-oxide free radical moiety. As used herein, the SOD mimic component itself is not intended to be a group capable of donating nitric oxide. Further, the SOD mimic component is provided in addition to the steroid component of the multifunctional steroid compound.

The multifunctional steroid compounds described herein may include one or more SOD mimic component. In certain embodiments, the compounds as described herein may comprise more than one SOD mimic component, for example at least one, at least two, at least three or at least four SOD mimic components.

As used herein, the term “alkyl” includes branched or unbranched hydrocarbon chains, for example, including about 1 to about 5 carbons, or 1-10, 1-5,1-3 or 1-2 carbons, such as methyl, ethyl, n-propyl, iso-propyl, n-butyl, sec-butyl, iso-butyl, tert-butyl, octa-decyl and 2-methylpentyl. Alkyl may also include cyclic alkyl groups, for example, including about 5-8 carbons, such as cyclopentyl, cyclohexyl, cycloheptyl, or cycloctyl. Alkyl can be optionally substituted with one or more functional groups such as hydroxyl, bromo, fluoro, chloro, iodo, mercapto or thio, cyano, alkylthio, aryl, carboxyl, carbalkoyl, alkenyl, nitro, amino, alkoxyl, amido, an NO donor component, and the like in the form of substituted alkyl. A cyclic alkyl group may be substituted with a straight or branched chain alkyl group.

The term “aryl” includes a chain of carbon atoms which form at least one aromatic ring having for example between about 6-14 carbon atoms, such as phenyl, naphthyl, anthracenyl, and azulenyl.

The aryl optionally may be substituted with one or more functional groups such as hydroxyl, bromo, fluoro, chloro, iodo, mercapto or thio, cyano, cyanoamido, alkylthio, heterocycle, aryl, heteroaryl, carboxyl, carbalkoyl, alkyl, alkenyl, nitro, amino, alkoxyl, amido, NO donor components, and the like.

The term “heteroaryl” includes a ring system including one or more aromatic rings and containing one or more heteroatoms, N, O, or S, in the aromatic ring. Heteroaryl groups can be unsubstituted or may be substituted for example as described for alkyl and aryl groups. Examples of heteroaryl groups include, but are not limited to, pyridinyl, pyrazinyl, pyrimidinyl, benzothialozyl, pyrazolyl, benzoxazolyl, imidazolyl, pyrrolyl, thiadiazolyl, oxazolyl, isoxazolyl, pyridazinyl, triazolyl, thiazolyl, isothiazolyl, thiophenyl, furanyl, and quinolinyl.

In particular embodiments, the SOD mimic component may be a substituted N-oxide free radical, wherein the nitrogen of the substituted N-oxide free radical is within a 3-, 4-, 5-, 6- or 7-member ring, wherein the ring may be optionally substituted with, for example, straight or branched chain C₁-C₅ alkyl groups (e.g. methyl, ethyl or propyl), alkoxy groups, and groups capable of donating NO in a charged or neutral form as described herein. In certain embodiments, the ring containing the N-oxide free radical is 5- or 6-member.

The ring containing the nitrogen of the substituted N-oxide free radical is preferably substituted at positions alpha to the nitrogen of the N-oxide free radical. In particular embodiments the N-oxide free radical is fully substituted at positions alpha to the nitrogen of the substituted N-oxide free radical, and may optionally be substituted at other positions on the ring. Exemplary substituents for the alpha positions include alkyl, e.g., methyl, ethyl, or one or more carbon atom of the steroid component, e.g. a saturated carbon atom (see compounds 1-8). In one embodiment, the positions alpha to the nitrogen are disubstituted, e.g. with dimethyl groups. Exemplary substituents for other ring positions include NO donor components. The alkyl groups alpha to the nitroxide may be further substituted with NO donor components, e.g. as in structures 1e and 1f.

In certain other embodiments the ring comprising the nitrogen of the N-oxide free radical may also be substituted with an additional heteroatom, for example, —O— or —S—. (see structures 1a, 1b and 1d, below). Exemplary SOD mimics from which the SOD mimic components may be selected include, but are not limited to, substituted N-oxide free radicals such as substituted pyrrolidinyloxy free radicals (e.g. PROXYL), substituted piperidinyloxy free radicals (e.g. TEMPO), substituted oxazolidinyloxy free radicals (e.g. DOXYL), substituted oxazinyloxy free radicals, substituted thiazolidinyloxy free radicals and substituted thiazinyloxy free radicals.

The SOD mimic component may be a substituted oxazinyloxy N-oxide free radical (e.g. 1a, where X=O), or substituted thiazolidinyloxy free radicals (e.g. 1b, where X=S) or substituted thiazinyloxy free radical (e.g. 1a, where X=S). In particular embodiments, X is —S— or —O—. In other embodiments the SOD mimic component comprises a 5-member ring where X is —CH₂— (e.g. PROXYL).

In certain embodiments, the SOD mimics from which the SOD mimic component(s) may be selected may be a substituted piperidinyloxy free radical (e.g. TEMPO), substituted 3-pyrrolidin-1-yloxy free radical (e.g. PROXYL), or substituted oxazolidinyloxy free radical (e.g. DOXYL).

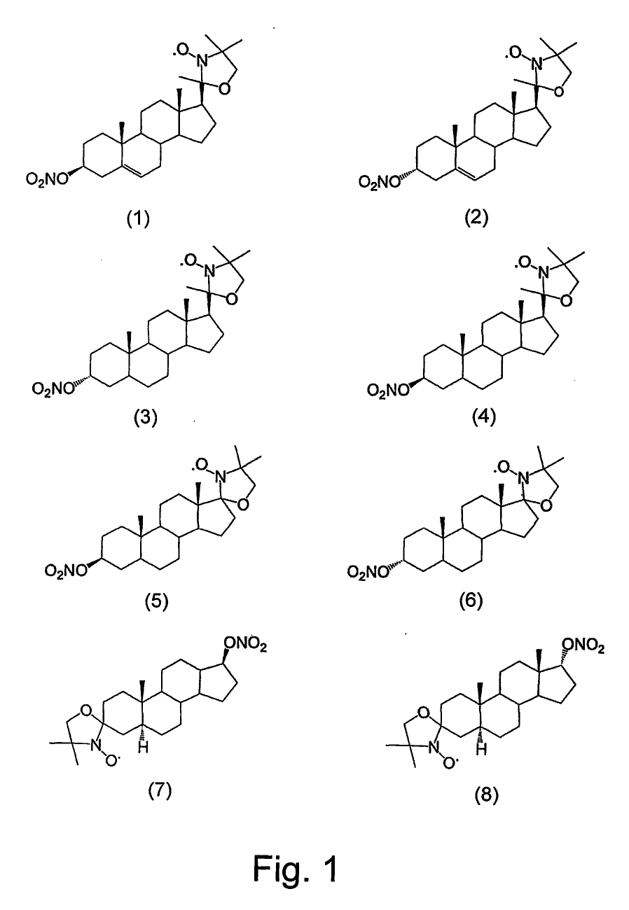

Examples of substituted N-oxide free radicals which may be incorporated into the multifunctional steroid compounds include substituted oxazolidinyloxy free radical moieties (1d, below). In structures 1a-1b, 1e, and 1f below, X is for example —S—, —CH₂— or —O—. The SOD mimic component may be linked to the steroid component for example, directly, or indirectly, via a linker (e.g. through an alkyl substituent group), or via a sharing of atoms. The TEMPO, DOXYL, and PROXYL moieties may share atoms with the steroid component, e.g., compounds 1-8, where one or more methyl group of the “DOXYL” exist as saturated carbons within the steroid ring. 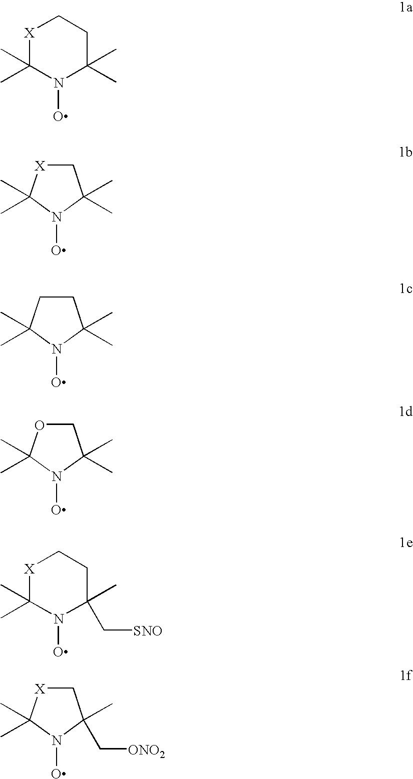
The ring containing the nitrogen of the substituted N-oxide free radical may be linked to the steroid component directly via a carbon-carbon bond, indirectly via a linker, or via sharing of atoms, for example via sharing of one or two carbons as, for example, in compounds 5-8 in FIG. 1.

In certain embodiments, the SOD mimic component, may also be independently substituted with one or more C₁-C₃ alkyl groups, hydroxy groups, amino groups (—NH₂), mercapto (—SH₂) and groups capable of donating NO in a charged or neutral form. In one embodiment, where the SOD mimic component includes a substituted N-oxide where the nitrogen of the substituted N-oxide is contained within a ring, the N-oxide-containing ring may be substituted at a position for example either annular (attached to the ring) or non-annular to the ring. For example, as in structures 1e and 1f, above, an alkyl substituent may be further substituted by an NO donor (non-annular substitution of the N-oxide-containing ring).

The multifunctional steroid compound may include one or more of the same or different SOD mimic components. In particular embodiments, the multifunctional steroid compound includes one, two, or three SOD-mimic components, which may be independently chosen.

Steroids

The steroid component of any of a variety of steroids used in the treatment of respiratory and other conditions in which treatment with steroids is indicated can be present in the multifunctional steroid compounds. Steroids include naturally occurring steroids and synthetic analogues thereof. In one embodiment, a known steroid (including steroids designed as analogues), is provided in modified multifunctional form and includes a nitric oxide donor component and a SOD mimic component. In some embodiments, the steroid is capable of exerting an anti-inflammatory effect through the reduction in concentration, distribution, chemoattraction, and function of peripheral leukocyte and inhibition of phospholipase A2. Preferred are steroids which may be functionalized with NO donor components and SOD mimic components using reactive functional groups already present on the steroid.

Steroids are indicated in the treatment of a variety of conditions, such as not limited to: respiratory disorders (e.g., asthma, chronic bronchitis, bronchiectasis, bronchospasms, emphysema, Chronic Obstructive Pulmonary Diseases (COPDs), bronchial hyperreactivity, respiratory distress syndrome or Chronic Obstructive Airway Disease (COADs), the treatment of allergic conditions, arthritis (e.g. rheumatoid or osteo arthritis), autoimmune conditions (e.g. autoimmune destruction of erythrocytes, autoimmune hematologic disorders, systemic lupus erythematosus, graft-vs.-host disease, etc.), cerebral edema, chronic adrenal insufficiency, congenital adrenal hyperplasia, gastrointestinal diseases, hepatic diseases, inflammatory bowel disease, malignancies, multiple sclerosis, neoplastic disease, ocular diseases, ophthalmic disorders, transplantation including bone marrow and organ transplantation, skin conditions (e.g. psoriasis, contact dermatitis, atopic dermatitis, exfoliative dermatitis, acne, hirsutism, erythema nodosum, inflamed cysts, discoid lupus, bullous diseases, collagen vascular diseases, sarcoidosis, Sweet's disease), renal disease, rheumatic disorders, sarcoidosis, systemic dermatomyositis, cancer, and thrombocytopenia.

The use of steroids for the treatment of the above-listed conditions are known to those of skill in the art (see, for example Goodman & Gillman, supra; Remington: The Science and practice of Pharmacy 20th Ed. (2000) Lippincott Williams and Wilkins, Ed. K. E. Hoover, Merck Index; Sanders et al. Am. J. Respir. Crit. Care. Med., (1995) 151: 1725-33) and the use of the multifunctional steroid compounds described herein in the treatment of these conditions has the benefit of increasing the efficacy of the treatment while decreasing the side effects associated with steroid treatment, and lowering toxicity.

The multifunctional steroid compounds of the present invention may also be employed in the treatment of conditions associated with endothelial dysfunction or oxidative stress including diabetes mellitus, cardiovascular diseases (such as ischaemic heart disease, angina pectoris, myocardial infarction, congestive heart failure, atherosclerosis, hypertension (e.g., pulmonary, systemic, ocular or pregnancy-induced) and arrhythmia), respiratory disorders (e.g., asthma, chronic bronchitis, bronchiectasis, bronchospasms, emphysema, Chronic Obstructive Pulmonary Diseases (COPDs), bronchial hyperreactivity, respiratory distress syndrome or Chronic Obstructive Airway Disease (COADs)), trauma, shock (hypovolumic, neurogenic or septic), neurotoxicity, neurodegenerative and neurological disorders (including Alzheimer and Parkinson's diseases, amyotrophic lateral sclerosis, multiple sclerosis, convulsive (seizure) disorders, AIDS-dementia and disorders which involve processes of learning, olfaction, nociception and memory), disorders of gastric acid and other secretory and peristaltic functions of the alimentary system (including relaxation and peristalsis of the intestinal tract (including sphincters)), drug and disease-induced neuropathies and nephropathies, pathological (premature) and physiological uterine contractions, migraine, sinus tachycardia, the symptoms of hyperthyroidism, cellular defense impairment, acute and chronic inflammatory conditions, diabetes mellitus (including the complications thereof, e.g. hypercholestemia, hypertension, atherosclerosis (e.g., arteriosclerosis) or Reaven's Syndrome, otherwise known as Syndrome-X), vasculitis, arteritis (e.g., temporal arteritis), endothelial dysfunction-induced diseases, insulin-resistance and glucose intolerance in diabetes, ischemia-reperfusion tissue injury, chemotaxis and phagocytic impairment in immunological disorders, aging and aging-mediated changes (e.g., premature balding, senescence-associated changes in skin and appearance), cerebrovascular diseases, thyrotoxicosis, aggregation disorders, fertility conditions and reproductive disorders (e.g., menopause, ovarian dysfunction, testicular dysfunction, penile erection and the treatment of male impotence). The compounds of the present invention can also be used in the treatment of allergic conditions, arthritis (e.g. rheumatoid or osteo arthritis), autoimmune conditions (e.g. autoimmune destruction of erythrocytes, autoimmune hematologic disorders, systemic lupus erythematosus, graft-vs.-host disease, etc.), cerebral edema, chronic adrenal insufficiency, congenital adrenal hyperplasia, gastrointestinal diseases, hepatic diseases, inflammatory bowel diseases (e.g., Crohn's disease and ulcerative colitis), malignancies, multiple sclerosis, neoplastic disease, ocular diseases, ophthalmic disorders (e.g., cataracts, retinopathy, glaucoma, corneal disease, etc.), transplantation including bone marrow and organ transplantation, skin conditions (e.g. psoriasis, contact dermatitis, atopic dermatitis, exfoliative dermatitis, acne, hirsutism, erythema nodosum, inflamed cysts, discoid lupus, bullous diseases, collagen vascular diseases, sarcoidosis, Sweet's disease), renal disease, rheumatic disorders, sarcoidosis, systemic dermatomyositis, cancer, and thrombocytopenia.

The use of the multifunctional steroid compounds described herein may be of particular use in the treatment of allergic conditions, including skin conditions, for example, psoriasis, contact dermatitis, atopic dermatitis; multiple sclerosis; inflammatory bowel disease; fertility conditions and reproductive conditions, for example, menopause, ovarian dysfuntion, testicular dysfunction; and respiratory disorders, as, for example, asthma, COPD, ARDS, etc.

Steroids (including designed analogues) are classed as corticosteroids, including glucocorticosteroids and mineralosteroids, and hormones. Hormonal steroids can be further classed as estrogens (e.g. estradiol), progesterones, or androgens (e.g., testosterone). There are both designed and naturally occurring anologues of these steroids which are contemplate within the scope of the present invention. Steroids can be additionally categorized as low, intermediate and high potency. Those containing an aromatic ring structure are generally higher potency than those without an aromatic ring. Similarly, those containing halogens are also usually of higher potency. Steroid with both an aromatic ring and a halogen atom have the highest potency. (Goodman & Gilman's The Pharmaceutical Basis of Therapeutics, Ed. Hardman, J. G. and Limbird, L. E., 10th Ed., 2001, McGraw-Hill, Medical Publishing Division). In particular embodiments, the multifunctional steroid compounds contain halogenated aromatic steroids as the steroid component. In other embodiments the steroid component is an aromatic non-halogenated steroid. In certain other embodiments, the steroids are chosen from the classes of corticosteroids (including glucocorticosteroids), mineralosteroids or hormones.

Exemplary steroids from which the streroid component is selected include steroids (including designed analogues) used in the treatment of respiratory and other disorders, such as corticosteroids (e.g. beclamethasone, triamcinolone, flunisolide, fluticasone, budesonide); and glucocorticoids (e.g. 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone-25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

Other exemplary steroids from which the steroid component of the multifunctional steroid compound may be selected include the hormone steroids including, estrogens, progesterones and androgens, particularly estradiol, testosterone and progesterone, and designed and natural analogues thereof. Where the steroid is a hormonal (also referred to a “sex hormone”) steroid, the multifunctional steroid compound may be of particular use in the treatment of reproductive disorders and fertility conditions, such as, menopause, ovarian dysfunction, and male impotence (e.g. testicular dysfunction). These multifunctional compounds may also be used in the treatment of premature baldness.

Steroids and their uses in treatment of respiratory and other disorders may also be described in the art, for example, Harrison's Principles of Internal Medicine, 13th Ed., Vol. 2, Ch. 335, pp:1973-1975; Holland & Taylor (1991) J. Fam. Pract. 31:512-519; Skorodin (1993) Arch. Inter. Med. 153:814-824; Swartz et al. 1(1978) Drugs 16:238-255; Goodman & Gilman's The Pharmaceutical Basis of Therapeutics, Ed. Hardman, J. G. and Limbird, L. E., 10th Ed., 2001, McGraw-Hill, Medical Publishing Division.

In particular embodiments, the steroid from which the steroid component is selected is androsterone, epiandrosterone, progesterone, testosterone, pregnenolone, cortisone, hydrocortisone, dexamethasone, prednisone, or prednisolone. In other embodiments, the steroid is androsterone, epiandrosterone, progesterone, testosterone, pregnenolone, cortisone, hydrocortisone, dexamethasone, prednisone, prednisolone, beclomethasone or budesonide.

In certain other embodiments the steroid may be beclomethasone, budesonide, fluticasone, mometasone, dexamethasone, clobetasone, or betamethasone.

Exemplary steroids are compounds 17-23 and compounds shown in below.

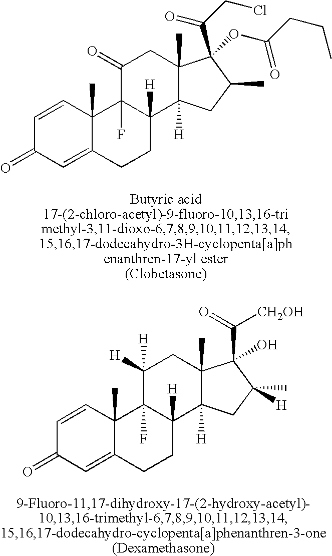

Multifunctional Steroid Compounds Comprising a Nitric Oxide Donor and SOD Mimic

The multifunctional steroid compounds described herein are characterized in comprising at least one NO donor component, at least one superoxide dismutase (SOD) mimic component and a steroid component. The compounds may include at least one NO donor component and at least one SOD mimic component linked to a steroid component. The term “linked” as used herein is intended to include direct or indirect linkages and shared atoms between any of the NO donor component, SOD mimic component and steroid component. The components may be linked in any order, for example, the SOD mimic component may be linked to both the NO donor component and the steroid component, or the SOD mimic component may be linked only to the steroid component while the steroid component is also linked to the NO donor component.

Also included within the scope of the invention are salts of the compounds disclosed herein and stereoisomers thereof. The compounds of the present invention contain one or more asymmetric atoms and may exist in diastereomeric, racemic and optically active forms. All such compounds and compositions comprising these compounds are contemplated to be within the scope of this invention. Therefore, where a compound is chiral, the separate enantiomers, substantially free of the other, are included within the scope of the invention. Thus, one enantiomer may be in, for example, 95% or more purity. Further included are all mixtures of enantiomers or diastereomers.

Optically active forms of the compounds can be prepared using any method known in the art, including by resolution of the racemic form by recrystallization techniques, by chiral synthesis, extraction with chiral solvents, or by chromatographic separation using a chiral stationary phase. Examples of methods to obtain optically active materials include transport across chiral membranes, a technique whereby a racemate is placed in contact with a thin membrane barrier. The concentration or pressure differential causes preferential transport across the membrane barrier. Separation occurs as a result of the non-racemic chiral nature of the membrane which allows only one enantiomer of the racemate to pass through. Chiral chromatography, including simulated moving bed chromatography, is used in one embodiment. A wide variety of chiral stationary phases are commercially available.

Steroids (including designed analogues) are available commercially as either α or β enantiomerically pure products. Regardless of the stereochemistry, the steroid will have a steroid function, such as an anti-inflammatory activity, however, the stereochemistry of the particular steroid component may affect the characteristics of binding of the steroid to the receptor and therefore may concomitantly have an effect on the potency of the steroid. All stereoisomers are within the scope of this invention, including those disclosed herein.

Since superoxide anion is an available and continuously-formed by-product generated through normal metabolic processes, and since its elimination is mediated either by dismutation by the enzyme SOD or via its reaction with NO to form the potentially hazardous peroxynitrite, without being limited to any theory, the compounds are believed to be capable of simultaneously and favorably affecting both components; the NO and O₂⁻. By virtue of the steroid activity, NO donation and superoxide scavenging properties being simultaneously delivered by the same molecule, the compounds of the present invention can increase the level of NO and reduce levels of superoxide thereby avoiding high levels of peroxynitrite and oxidant metabolites thereof and consequently increasing the effectiveness of the steroid active agent.

Multifunctional steroid compounds of formulae (4) and (5) are provided by this invention. In preferred embodiments of this invention, multifunctional steroid compounds of formulae I-VI are provided. The beneficial therapeutic effects of compounds of these formulae may, without being limited by theory, be attributed to their simultaneous multi-mechanistic actions as steroids (e.g., immunosuppressant, anti-inflammatory, and/or anti-allergic), SOD mimics (antioxidant and anti-inflammatory that provide additional cellular protection), and as NO donors (antioxidant, anti-proliferative, cellular protectant with potent smooth muscle relaxing properties). These properties are most needed for adequate prevention and treatment of acute episodes of inflammatory disorders involving allergy, immune stimulation and proliferation, depletion of natural antioxidants, and bronchoconstriction, as is the case in, for example, asthma, as well as other pathologies involving immunoreaction, inflammation, oxidative stress and free radical injury.

In certain examples of the multifunctional steroid compounds, the compounds as described herein, e.g. compounds of formulae I-VI, have at least one NO donor component and at least one SOD mimic component (e.g., substituted N-oxide free radical). In particular embodiments, the ratio of NO donor components:substituted N-oxide free radical components is 1:1, 2:1 or 1:2. In certain embodiments there are one, two, three or four NO donor components. In particular embodiments there are one, two or three SOD mimic components. Where there is more than one SOD mimic component, the SOD mimic components may be the same or different. Where there is more than one NO donor component, the NO donor components may be the same.

Compounds described herein in one embodiment may include the core ring structures shown in structures 2a-2d below. The core ring structure may contain one or two double bonds as shown in structures 2b-2d. Or as shown in structure 2a, the core ring structure may not contain any double bonds. The core ring structure and the position of particular keto or hydroxyl functional groups on the core ring structure will vary depending on the steroid from which the steroid component is derived.

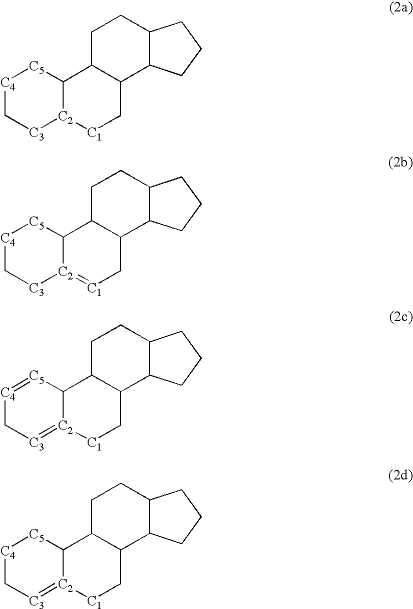

Optionally, in particular embodiments, the steroid core ring structure depicted above may be modified with regard to the position of the double bond(s) or by modification of functional groups prior to modification to include the NO donor and SOD mimic components. Such modifications prior to the attachment of NO donor and SOD mimic components are well within the skill of those in the art.

In one embodiment, compounds of formulae I (Ia-Id) are provided, as shown below.

In certain embodiments of formulae I (Ia-Id):

R² is —H, —ONO, —ONO₂, —SNO, —OH, —CH₃, —NONOate, or —OC(O)R⁸;
wherein R⁸ is C₁-C₅ alkyl (e.g. C₁-C₃, methyl or ethyl), or 5- or 6-member heteroaryl (e.g. furan, pyrrole, thiazole, oxazole, thiophene, pyridine, imidazole, or pyran);

R³ is —H, —OH, or —CH₃; or
R² and R³ together form a heterocyclic ring;

R⁴ is —H or halogen (e.g., —F, —I, —Br or —C);
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups (e.g. C₁-C₃, methyl or ethyl);

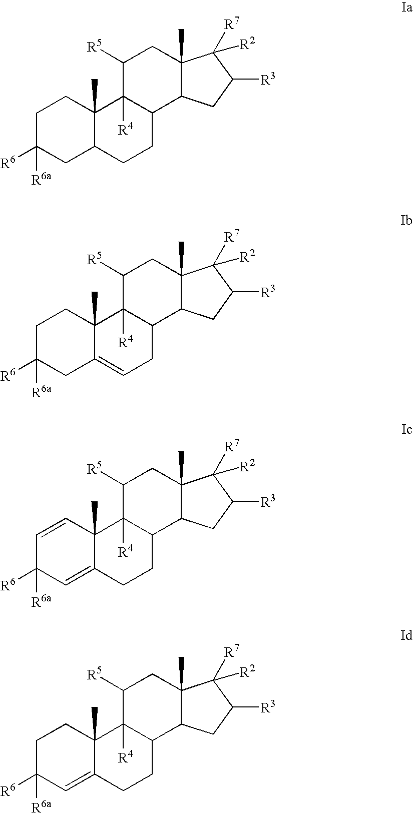

R⁶ is ═O, —ONO, —ONO₂, —SNO, —NONOate and R^{6A}, if present, is —H, or R⁶ and R^{6A} together form a substituted N-oxide free radical, wherein the nitrogen of the N-oxide group in the substituted N-oxide free radical is within a 5- or 6-member ring substituted by one or more independently selected C₁-C₅ alkyl groups (e.g. C₁-C₃, methyl or ethyl),
wherein the alkyl substituent group may be further independently substituted by an NO donor component (e.g. —ONO₂, —SNO), or OC(O)R¹²,
   wherein R¹² is C₁-C₅ alkyl (e.g. C₁-C₃), or 5- or 6-member heteroaryl (e.g. furan, pyrrole, thiazole, oxazole, thiophene, pyridine, imidazole, or pyran);

R⁷ is —H, —ONO, —ONO₂, —SNO, —NONOate, or a substituted N-oxide free radical, wherein the nitrogen of the N-oxide group in the substituted N-oxide free radical is within a 5- or 6-member ring optionally substituted by —OCOCH₂-PEG (e.g., PEG molecular weight from about 100 to about 4000 daltons), and/or one or more independently selected C₁-C₅ alkyl groups (e.g. C₁-C₃, methyl or ethyl),
wherein the alkyl substituent group may be further independently substituted by an NO donor component (e.g. —ONO₂, —SNO), —SR¹¹-halogen, or —OC(O)R¹³,
   wherein R¹¹ is C₁-C₅ alkyl (e.g. C₁-C₃) and halogen may be —F, —Cl, —I, —Br;
   wherein R¹³ is C₁-C₅ alkyl (e.g. C₁-C₃), or 5- or 6-member heteroaryl (e.g. furan, pyrrole, thiazole, oxazole, thiophene, pyridine, imidazole, or pyran), or
R² and R⁷ together form a substituted N-oxide free radical; and

wherein at least one of R², R⁵, R⁶, or R⁷ comprises an NO donor; and

wherein at least one of R⁵, R⁶, or R⁷ comprises a substituted N-oxide free radical.

In certain embodiments of formulae I (Ia-Id):

R² is —H, or —ONO₂;

R³ is —H, —OH, or —CH₃;

R⁴ is —H, —F or —Cl;

R⁵ is —H, ═O, or —ONO₂;

R⁶ is ═O, or —ONO₂ and R^{6A}, if present, is —H, or R⁶ and R^{6A} together form a substituted N-oxide free radical, e.g. substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical; and

R⁷ is —H, —ONO₂ or a substituted N-oxide free radical, e.g. substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical, or R² and R⁷ together form a substituted N-oxide free radical, and

wherein at least one of R², R⁵, R⁶, or R⁷ comprises an NO donor; and

wherein at least one of R⁵, R⁶, or R⁷ comprises a substituted N-oxide free radical.

In one example of formulae Ia, and Ib,
R², R³, R⁴, and R⁵ are —H;
R⁶ is —ONO₂ and R^{6A}, if present, is —H, or R⁶ and R^{6A} together form a substituted N-oxide free radical, e.g. substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical; and
R⁷ is —ONO₂ or a substituted N-oxide free radical, e.g. substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical, and

wherein at least one of R⁶, or R⁷ comprises an NO donor; and

wherein at least one of R⁶, or R⁷ comprises a substituted N-oxide free radical.

In one embodiment of Formula Ia, and Ib,
R², R³, R⁴, R⁵ and R^{6A} are —H;
R⁶ is —ONO₂; and
R⁷ is a substituted N-oxide free radical, e.g. substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical.

In one embodiment of Formula Ib, Ic, and Id,
R² and R⁵ are —ONO₂;
R³ is —H or CH₃;
R⁴ is —H, —F or —Cl;
R⁵ is ═O or —ONO₂;
R^{6A}, if present, is —H; and
R⁷ is a substituted N-oxide free radical, e.g. substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical.

In other examples of formulae Ib, Ic, and Id,
R² and R⁵ are —ONO₂;
R³ is —CH₃;
R⁴ is —F or —Cl;
R⁶ is ═O or —ONO₂;
R^{6A}, if present, is —H; and
R⁷ is a substituted N-oxide free radical, e.g. substituted pyrrolidinyloxy N-oxide free radical, substituted piperidinyloxy N-oxide free radical, substituted oxazolidinyloxy N-oxide free radical, or substituted oxazinyloxy N-oxide free radical.

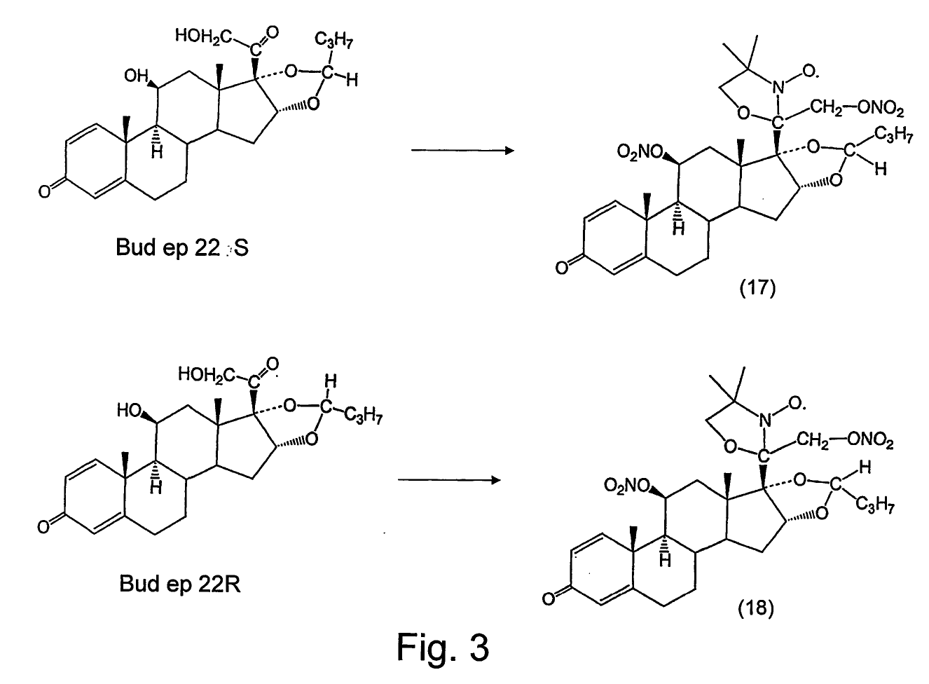

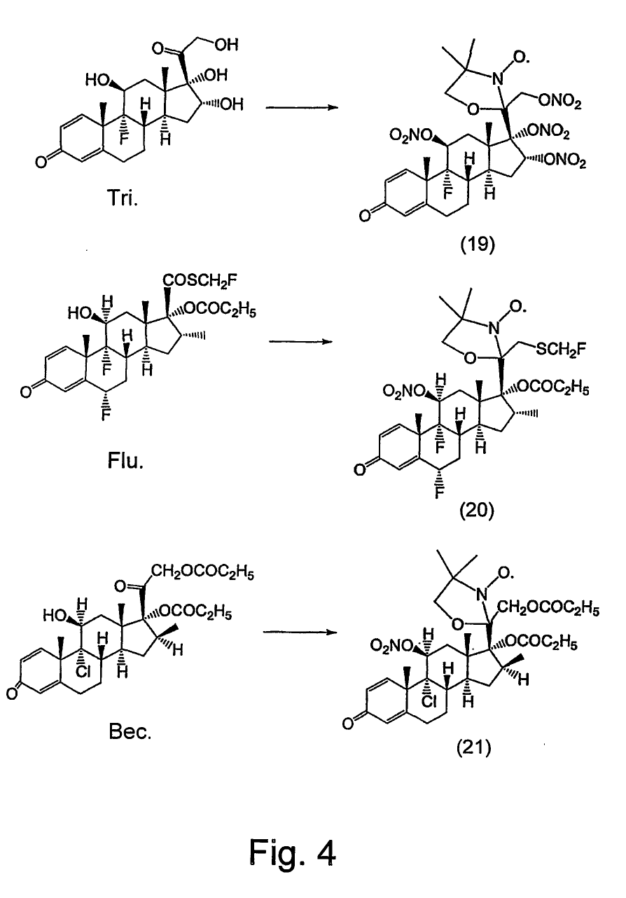

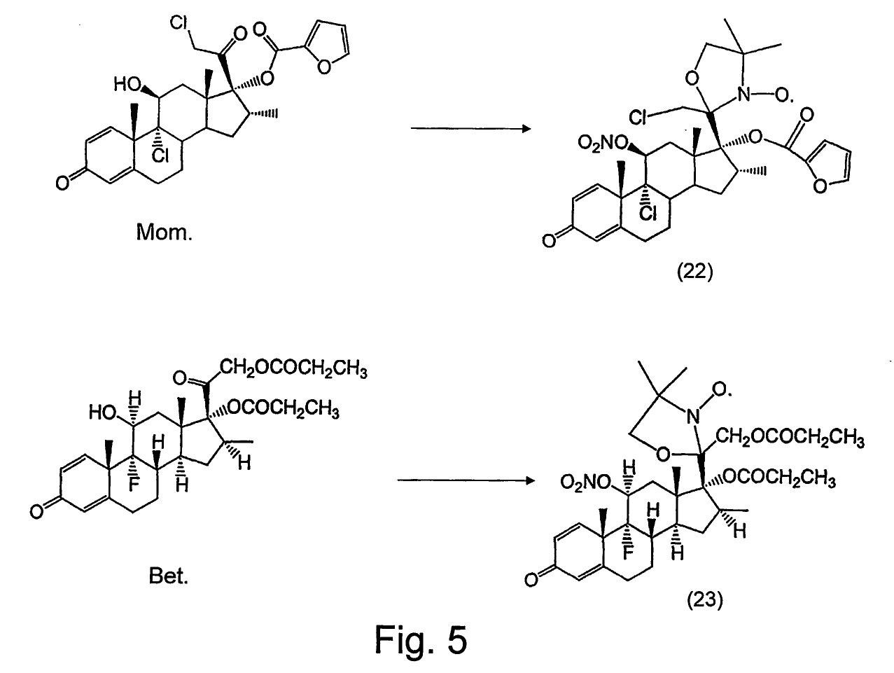

In particular embodiments of formulae I (Ia-Id), the multifunctional steroid compounds are as shown in FIGS. 1, 2, 3, 4, 5, 6 or 7. In certain embodiments of formulae I (Ia-Id), R² is —H, —ONO, —ONO₂, or —SNO, e.g., —H, or —ONO₂.

In one embodiments of formulae I (Ia-Id), R³ is —H.

In particular embodiments of formulae I (Ia-Id), R⁴ is —H, —Cl or —F, e.g., —H or —F.

In certain embodiments of formulae I (a-Id), R⁵ is —H, —ONO, —ONO₂, or —SNO. In other embodiments, R⁵ is —H, or —ONO₂.

In some embodiments of formulae I (Ia-Id), R⁶ is ═O, —ONO₂, —SNO, or a substituted N-oxide free radical, e.g., ═O, or —ONO₂.

In some embodiments of formulae I (Ia-Id), R⁷ is —H, —ONO₂, —ONO, or a substituted N-oxide free radical, e.g., —H, or —ONO₂.

In some embodiments of formulae I (Ia-Id), R⁸ is C₁-C₃ alkyl, e.g., methyl or ethyl.

In one embodiment, R⁸ is a 5- or 6-member heteroaryl (e.g. furan, pyrrole, thiazole, oxazole, thiophene, pyridine, imidazole, or pyran

In some embodiments of formulae I (Ia-Id), R¹², and R¹³, may be, independently, selected C₁-C₃ alkyl (e.g. methyl, ethyl or butyl) or furan.

In some embodiments of formulae II (Ia-Id), R¹¹ may be, independently, selected C₁-C₃ alkyl (e.g. methyl, ethyl or butyl) and halogen may be —F.

In particular examples of formulae I (Ia-Id), where R⁵, R⁶, or R⁷ includes a substituted N-oxide free radical where the nitrogen of the N-oxide group of the substituted N-oxide free radical is within a 5- or 6-member ring, the one or more substituted N-oxide free radicals may be, independently, for example, substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical (which are also described by structures 3a and 3b, below).

In other examples of formulae I (Ia-Id), where R⁵, R⁶/R^{6A}, or R⁷ include a substituted N-oxide free radical where the nitrogen of the N-oxide group of the substituted N-oxide free radical is within a 5- or 6-member ring, the one or more substituted N-oxide free radicals may independently be, for example a substituted 3-oxazolidinyloxy free radical.

In other examples of formulae I (Ia-Id), R², R⁵ and R⁶ may be, independently, —H, —NO₂ or —SNO.

In another embodiment, compounds of formulae II (IIa-IId) below are provided:

In one embodiment of formulae II (IIa-IId):

R² is —H, —ONO, —ONO₂, NO, —OH, —CH₃, —NONOate, or —OC(O)R⁸;
wherein R⁸ is C₁-C₅ alkyl (e.g. C₁-C₃, methyl or ethyl), or 5- or 6-member heteroaryl (e.g. furan, pyrrole, thiazole, oxazole, thiophene, pyridine, imidazole, or pyran);

R³ is —H, —OH, or —CH₃; or
R² and R³ together form a heterocyclic ring;

R⁴ is —H or halogen (e.g., —F, —I, —Br or —Cl);

R⁵ is —H, ═O, —ONO, —ONO₂, —SNO, —NONOate or a substituted N-oxide free radical; 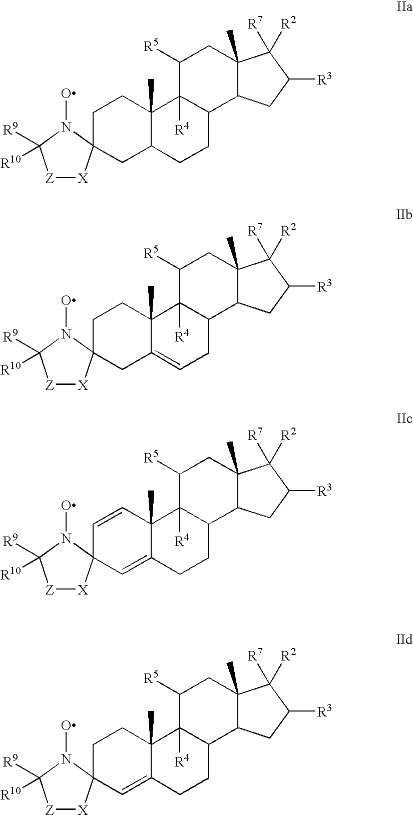
wherein the nitrogen of the N-oxide group in the substituted N-oxide free radical is within a 5- or 6-member ring substituted by one or more independently selected C₁-C₅ alkyl groups (e.g. C₁-C₃, methyl or ethyl);

R⁷ is —H, —ONO, —ONO₂, —SNO, —NONOate, or a substituted N-oxide free radical; wherein the nitrogen of the N-oxide group in the substituted N-oxide free radical is within a 5- or 6-member ring substituted by —OCOCH₂-PEG (e.g., PEG molecular weight from about 100 to about 4000 daltons), and/or one or more independently selected C₁-C₅ alkyl groups (e.g. C₁-C₃);
wherein the alkyl substituent group may be further independently substituted by an NO donor component (e.g. —ONO₂, —SNO), —SR¹¹-halogen, or —OC(O)R¹³;
   wherein R¹¹ is C₁-C₅ alkyl (e.g. C₁-C₃) and halogen may be —F, —Cl, —I, —Br;
wherein R¹³ is C₁-C₅ alkyl (e.g. C₁-C₃), or 5- or 6-member heteroaryl (e.g. furan, pyrrole, thiazole, oxazole, thiophene, pyridine, imidazole, or pyran);

R⁹ and R¹⁰ are independently, linear or branched C₁-C₅ alkyl groups (e.g. C₁-C₃, methyl or ethyl), or substituted linear or branched C₁-C₅ alkyl groups (e.g., C₁-C₃, methyl or ethyl) wherein the alkyl group is independently substituted by —ONO, —ONO₂, —SNO, —NONOate (e.g. —ONO, —ONO₂, —SNO) or —OC(O)R¹⁴;
wherein R¹⁴ is C₁-C₅ alkyl (e.g., C₁-C₃, methyl or ethyl), or 5- or 6-member heteroaryl (e.g. furan, pyrrole, thiazole, oxazole, thiophene, pyridine, imidazole, or pyran);
X is —CH₂—, —O— or —S—;
Z is —CH₂— or —CH₂—CH₂—; and
wherein at least one of R², R⁵, R⁷, R⁹ or R¹⁰ comprises an NO donor.

In one embodiment of formulae II (IIa-IId),

R² is —H, or —ONO₂;

R³ is —H, —OH, or —CH₃;

R⁴ is —H, —F or —Cl;

R⁵ is —H, ═O, or —ONO₂;

R⁷ is —ONO₂, or a substituted N-oxide free radical, e.g. substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical;

R⁹ and R¹⁰ may be, independently, selected C₁-C₂ alkyl (e.g., methyl or ethyl);

X is —O— or —CH₂—; and

Z is —CH₂—; and

wherein at least one of R², R⁵, or R⁷ comprises an NO donor.

In one embodiment of formulae II (IIa-IId),

R² and R⁵ are —H, —SNO or —ONO₂;

R³ and R⁴ are —H;

R⁷ is —ONO₂ or a substituted N-oxide free radical, e.g. substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical;

X is —O—;

Z is —CH₂—; and

R⁹ and R¹⁰ may be, independently, C₁-C₂ alkyl alkyl (e.g., methyl or ethyl);

where at least one of R², R⁵ and R⁷ comprises an NO donor.

In one embodiment of formulae II (IIa-IId),

R², R³, R⁴, and R⁵ are —H;

R⁷ is —ONO₂;

X is —O—;

Z is —CH₂—; and

R⁹ and R¹⁰ may be, independently, C₁-C₂ alkyl (e.g., methyl or ethyl).

In one embodiment of formulae II (IIa-IId), if R⁷ is —ONO₂, then R² is —H.

In another embodiment of formulae II (IIa-IId), if R² is —ONO₂, then R⁷ is a substituted N-oxide free radical.

Particular examples of formulae II (IIa) include compounds 7 and 8, as shown in FIG. 1.

In certain embodiments of formulae II (IIa-IId), R² is —H, —ONO, —ONO₂, or —SNO, e.g., —H, or —ONO₂.

In one embodiment of formulae II (Ia-IId), R³ is —H.

In particular embodiments of formulae II (IIa-IId), R⁴ is —H, —Cl or —F, e.g. —H or —F.

In certain embodiments of formulae II (IIa-IId), R⁵ is —H, —ONO, —ONO₂, or —SNO. In other embodiments, R⁵ is —H, or —ONO₂.

In some embodiments of formulae II (IIa-IId), R⁷ is —H, —ONO₂, —ONO, or a substituted N-oxide free radical, e.g., —H, or —ONO₂.

In some embodiments of formulae II (IIa-IId), R⁸ is C₁-C₃ alkyl, e.g., methyl or ethyl.

In one embodiment, R⁸ is a 5- or 6-member heteroaryl (e.g. furan, pyrrole, thiazole, oxazole, thiophene, pyridine, imidazole, or pyran.

In some embodiments of the formulae II (IIa-IId), R⁹ and R¹⁰ are independently, C₁-C₃ alkyl, e.g. methyl or ethyl.

In some embodiments of formulae II (IIa-IId), R¹², and R¹³, may be, independently, selected C₁-C₃ alkyl (e.g. methyl, ethyl or butyl) or furan.

In some embodiments of formulae II (IIa-IId), R¹¹ may be, independently, selected C₁-C₃ alkyl (e.g. methyl, ethyl or butyl) and halogen may be —F.

In particular embodiments of formulae II (IIa-IId), Z is —CH₂—.

In some embodiments of formulae II (IIa-IId), X is —O—, —CH₂—, or —S—, e.g., —O— or —CH₂—.

In particular examples of formulae II (IIa-IId), where R⁵ or R⁷ includes a substituted N-oxide free radical where the nitrogen of the N-oxide group of the substituted N-oxide free radical is within a 5- or 6-member ring, the one or more substituted N-oxide free radicals may independently be substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical and substituted thiazinyloxy N-oxide free radical (which may also be described by formulae 3a-3b, above). In other examples of formulae II (IIa-IId), where R⁵ or R⁷ includes a substituted N-oxide free radical where the nitrogen of the substituted N-oxide free radical is within a 5- or 6-member ring, the one or more substituted N-oxide free radicals may be, independently, substituted 3-oxazolidinyloxy free radical. In certain embodiments of Formula II (IIa-IId), R² and R⁵ may be, independently, —H, —ONO₂ or —SNO.

Further, compounds of formulae III (IIIa-IIId) are provided (below).

In one embodiment of formulae III (IIIa-IIId):

R¹ is —H, —OH, —OCOCH₂-PEG (e.g., PEG molecular weight from about 100 to about 4000 daltons); linear or branched C₁-C₂ alkyl, linear or branched C₁-C₂ alkyl substituted by —ONO, —ONO₂, —SNO, or —NONOate, (e.g. —ONO, —ONO₂, or —SNO) —SR¹¹-halogen, or —OC(O)R¹⁵;
wherein R¹¹ is C₁-C₅ alkyl (e.g. C₁-C₃) and halogen may be —F, —Cl, —I, —Br; 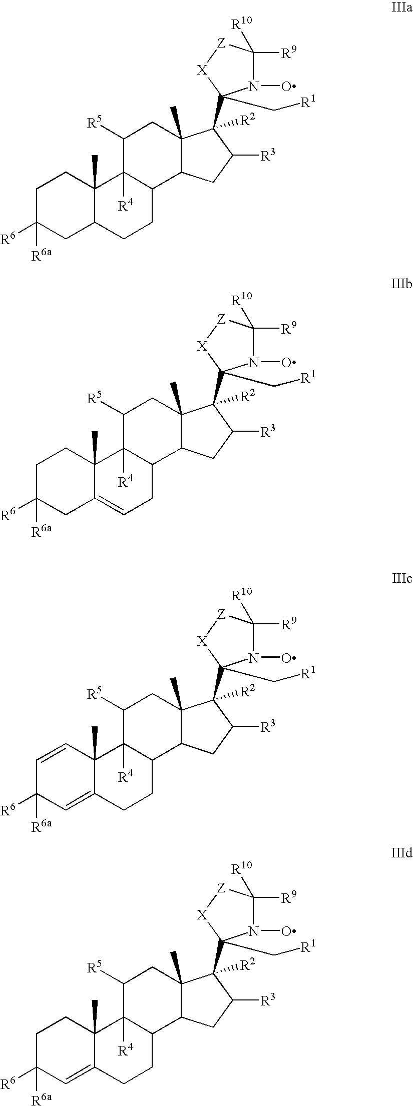
wherein R¹⁵ is C₁-C₅ alkyl (e.g., C₁-C₃, methyl, ethyl);

R² is —H, —ONO, —ONO₂, —SNO, —OH, —CH₃, —NONOate, or —OC(O)R⁸;
wherein R⁸ is C₁-C₅ alkyl (e.g. C₁-C₃), or 5- or 6-member heteroaryl (e.g. furan, pyrrole, thiazole, oxazole, thiophene, pyridine, imidazole, or pyran);

R³ is —H, —OH, or —CH₃; or

R² and R³ together form a heterocyclic ring;

R⁴ is —H or halogen (e.g., —F, —I, —Br or —Cl);

R⁵ is —H, ═O, —ONO, —ONO₂, —SNO, —NONOate or a substituted N-oxide free radical; wherein the nitrogen of the N-oxide group in the substituted N-oxide free radical is within a 5- or 6-member ring substituted by one or more independently selected C₁-C₅ alkyl groups (e.g. C₁-C₃, methyl, ethyl);

R⁶ is ═O, —ONO, —NO₂, —SNO, —NONOate and R^{6A}, if present, is —H, or R⁶ and R^{6A} together form a substituted N-oxide free radical, wherein the nitrogen of the N-oxide group in the substituted N-oxide free radical is within a 5- or 6-member ring substituted by one or more independently selected C₁-C₅ alkyl groups (e.g. C₁-C₃, methyl, ethyl);
wherein the alkyl substituent group may be further substituted by an NO donor component (e.g. NO₂, —SNO), or —OC(O)R¹²,
   wherein R¹² is C₁-C₅ alkyl (e.g. C₁-C₃, methyl or ethyl), or 5- or 6-member heteroaryl (e.g. furan, pyrrole, thiazole, oxazole, thiophene, pyridine, imidazole, or pyran);
R⁹ and R¹⁰ are independently, linear or branched C₁-C₅ alkyl groups (e.g., C₁-C₃, methyl or ethyl), or substituted linear or branched C₁-C₅ alkyl groups (e.g., C₁-C₃, methyl or ethyl) wherein the alkyl group is independently substituted by —ONO, —NO₂, —SNO, —NONOate (e.g. —ONO, —ONO₂, —SNO) or —OC(O)R¹⁴,
   wherein R¹⁴ is C₁-C₅ alkyl (e.g., C₁-C₃, methyl or ethyl);

X is —CH₂—, —O— or —S—;

Z is —CH₂— or —CH₂—CH₂—; and

wherein at least one of R¹, R², R⁵, R⁶, R⁹ or R¹⁰ comprises at least one NO donor.

In certain embodiments of formulae III (IIIa-IIId) above,

R¹ is —H, —SNO, or —NO₂;

R² is —H, or —NO₂;

R³ is —H, —OH, or —CH₃;

R⁴ is —H, —F or —Cl;

R⁵ is —H, ═O, or —ONO₂;

R⁶ is ═O or —NO₂ and R^{6A}, if present, is —H, or R⁶ and R^{6A} together form a substituted N-oxide free radical, e.g. substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical;

R⁹ and R¹⁰ may be, independently, C₁-C₂ alkyl, e.g., methyl or ethyl;

X is —O— or —CH₂—; and

Z is —CH₂—; and

wherein at least one of R¹, R², R⁵, or R⁶ comprises at least one NO donor.

In one embodiment of formulae III (IIIa-IIId),

R¹ is —H or —ONO₂;

R², R³, R⁴, and R⁵ are —H;

R⁶ is —NO₂ and R^{6A}, if present, is —H, or R⁶ and R^{6A} together form a substituted N-oxide free radical, e.g. substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical;

R⁹ and R¹⁰ may be, independently, C₁-C₂ alkyl, e.g., methyl or ethyl;

X is —O— or —CH₂—; and

Z is —CH₂—; and

wherein at least one of R¹ or R⁶ comprises at least one NO donor.

In one embodiment of formulae IIIa-IIId,

R¹, R², R³, R¹, R⁵ and R^{6A} are —H;

R⁶ is —ONO₂;

R⁹ and R¹⁰ may be, independently, C₁-C₂ alkyl, e.g., methyl or ethyl;

X is —O—; and

Z is —CH₂—.

In one embodiment of formulae IIIb, IIIc, and IIId as shown above,

R¹ is —H or —ONO₂;

R² and R⁵ are —ONO₂;

R³ is —H or —CH₃;

R⁴ is —H, —F or —Cl;

R⁶ is ═O or —ONO₂;

R^{6A}, if present, is —H;

R⁹ and R¹⁰ may be, independently, C₁-C₂ alkyl, e.g., methyl or ethyl;

X is —O—; and

Z is —CH₂—.

In other examples of formulae IIIb, IIIc, and IIId,

R¹, R² and R⁵ are —ONO₂;

R³ is —CH₃;

R⁴ is —F or —Cl;

R⁶ is ═O or —ONO₂;

R^{6A}, if present, is —H;

R⁹ and R¹⁰ may be, independently, C₁-C₂ alkyl, e.g., methyl or ethyl;

X is —O—; and

Z is —CH₂—.

In some embodiments of formulae III (IIIa-IIId), the multifunctional steroid compounds may include compounds 1-4 and 9-23 in FIGS. 1, 2, 3, 4, and 5.

In one embodiment of formulae III (IIIa-IIId), R¹ is —H, —OH, —SNO, —ONO, or —ONO₂, e.g. —SNO or —ONO₂.

In certain embodiments of Formula I (Ia-Id), R² is —H, —ONO, —ONO₂, or —SNO, e.g., —H, or —ONO₂.

In one embodiments of Formula I (Ia-Id), R³ is —H.

In particular embodiments of formulae III (IIIa-IIId), R⁴ is —H, —Cl or —F, e.g., —H or —F.

In certain embodiments of formulae III (IIIa-IIId), R¹ is —H, NO, —ONO₂, or —SNO. In other embodiments, R⁵ is —H, or —ONO₂.

In some embodiments of formulae III (IIIa-IIId), R⁶ is ═O, —ONO₂, —SNO, or a substituted N-oxide free radical, e.g., ═O, or —ONO₂.

In some embodiments of formulae III (IIIa-IIId), R⁸ is C₁-C₃ alkyl, e.g., methyl or ethyl.

In one embodiment, R⁸ is a 5- or 6-member heteroaryl (e.g. furan, pyrrole, thiazole, oxazole, thiophene, pyridine, imidazole, or pyran

In some embodiments of the formulae III (IIIa-IIId), R⁹ and R¹⁰ are independently, C₁-C₃ alkyl, e.g. methyl or ethyl.

In some embodiments of formulae III (IIIa-IIId), R¹², R¹⁴ and R¹⁵, may be, independently, selected C₁-C₃ alkyl (e.g. methyl, ethyl or butyl) or furan.

In some embodiments of formulae III (IIIa-IIId), R¹¹ may be, independently, selected C₁-C₃ alkyl (e.g. methyl, ethyl or butyl) and halogen may be —F.

In particular embodiments of formulae III (IIIa-IIId), Z is —CH₂—.

In some embodiments of formulae III (IIIa-IIId), X is —O—, —CH₂—, or —S—, e.g., —O— or —CH₂—.

In particular examples of formulae III (IIIa-IIId), where R⁵ or R⁶ include a substituted N-oxide free radical where the nitrogen of the N-oxide group of the substituted N-oxide free radical is within a 5- or 6-member ring, the one or more substituted N-oxide free radicals may be, independently, substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical (which may also be described by formulae 3a-3b, above).

In other examples of formulae III (IIIa-IIId), where R⁵ or R⁶ include a substituted N-oxide free radical where the nitrogen of the N-oxide group of the substituted N-oxide free radical is within a 5- or 6-member ring, the one or more substituted N-oxide free radicals may be, independently, substituted 3-oxazolidinyloxy free radical.

In other examples of formulae III (IIIa-IIId), R², R¹ and R⁶ may be, independently, —H, —ONO₂ or —SNO.

In one embodiment, compounds of formulae IV (IVa-IVd) below, also are provided.

In certain embodiments of formulae IV (IVa-IVd):
R¹ is —H, —OH, —OCOCH₂-PEG (e.g., PEG molecular weight from about 100 to about 4000 daltons); linear or branched C₁-C₂ alkyl; linear or branched C₁-C₂ alkyl substituted by —ONO, —NO₂, —SNO, or —NONOate, (e.g. —ONO, —NO₂, or —SNO), —SR¹¹— halogen, or —OC(O)R¹⁵;
wherein R¹¹ is C₁-C₅ alkyl (e.g. C₁-C₃) and halogen may be —F, —Cl, —I, —Br;
wherein R¹⁵ is C₁-C₅ alkyl (e.g., C₁-C₃, methyl, ethyl);
R² is —H, —ONO, —NO₂, —SNO, —OH, —CH₃, —NONOate, or —OC(O)R⁸, 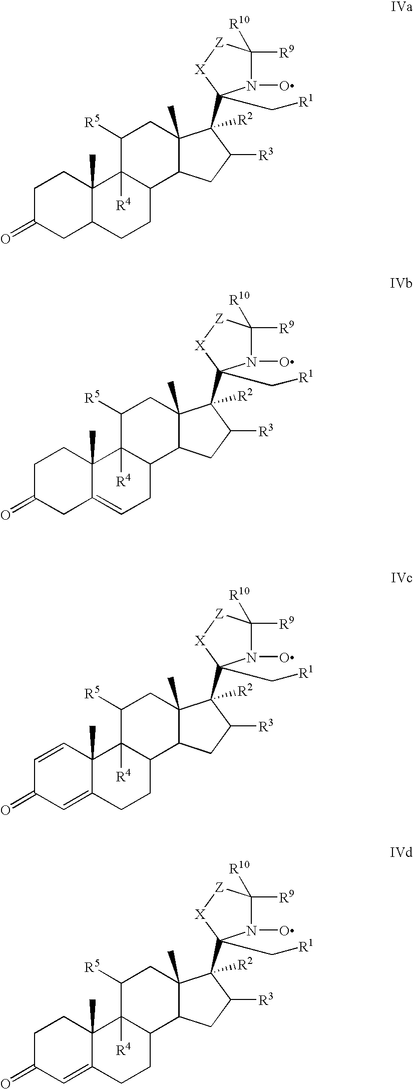
   wherein R⁸ is C₁-C₅ alkyl (e.g. C₁-C₃), or 5- or 6-member heteroaryl (e.g. furan, pyrrole, thiazole, oxazole, thiophene, pyridine, imidazole, or pyran);
R³ is —H, —OH, or —CH₃; or
   R² and R³ together form a heterocyclic ring;
R⁴ is —H or halogen (e.g., —F, —I, —Br or —C);
R⁵ is —H, ═O, —ONO, —ONO₂, —SNO, —NONOate or a substituted N-oxide free radical;
   wherein the nitrogen of the N-oxide group in the substituted N-oxide free radical is within a 5- or 6-member ring substituted by one or more independently selected C₁-C₅ alkyl groups (e.g. C₁-C₃, methyl or ethyl);
R⁹ and R¹⁰ are independently, linear or branched C₁-C₅ alkyl groups (e.g., C₁-C₃, methyl or ethyl), or substituted linear or branched C₁-C₅ alkyl groups (e.g., C₁-C₃, methyl or ethyl) wherein the alkyl group is independently substituted by —ONO, —ONO₂, —SNO, —NONOate (e.g. —ONO, —ONO₂, —SNO) or —OC(O)R¹⁴;
   wherein R¹⁴ is C₁-C₅ alkyl (e.g., C₁-C₃, methyl or ethyl);
X is —CH₂—, —O— or —S—;
Z is —CH or —CH₂—CH₂—; and
wherein at least one of R¹, R², R⁵, R⁹ or R¹⁰ comprises at least one NO donor.

In certain embodiments of formulae IV (IVa-IVd),

R¹ is —H, —SNO or —ONO₂;

R² is —H, or —ONO₂;

R³ is —H, —OH, or —CH₃;

R⁴ is —H, —F or —Cl;

R⁵ is —H, ═O, or —ONO₂;

R⁹ and R¹⁰ may be, independently, C₁-C₂ alkyl (e.g., methyl or ethyl);

X is —O— or —CH₂—; and

Z is —CH₂—; and
wherein at least one of R¹, R², and R⁵ comprises at least one NO donor.

In one embodiment of formulae IV (IVa-IVd),

R¹, R², and R⁵ are —H or —ONO₂;

R³ and R⁴ are —H;

R⁹ and R¹⁰ may be, independently, C₁-C₂ alkyl (e.g., methyl or ethyl);

X is —O— or —CH₂—; and

Z is —CH₂—; and

wherein at least one of R¹, R², and R⁵ comprises at least one NO donor.

In one embodiment of formulae IVb, IVc, and IVd,

R¹ and R² are —H or —NO₂;

R³ is —H or —CH₃;

R⁴ is —H, —F or —Cl;

R⁵ is —H, ═O or —ONO₂;

R⁹ and R¹⁰ may be, independently, C₁-C₂ alkyl (e.g., methyl or ethyl);

X is —O—; and

Z is —CH₂—; and

wherein at least one of R¹, R², and R⁵ comprises at least one NO donor.

In other examples of formulae IVb, IVc, and IVd,

R¹, R² and R⁵ are —ONO₂;

R³ is —CH₃;

R⁴ is —F or —Cl;

R⁹ and R¹⁰ may be, independently, C₁-C₂ alkyl (e.g., methyl or ethyl);

X is —O—; and

Z is —CH₂—.

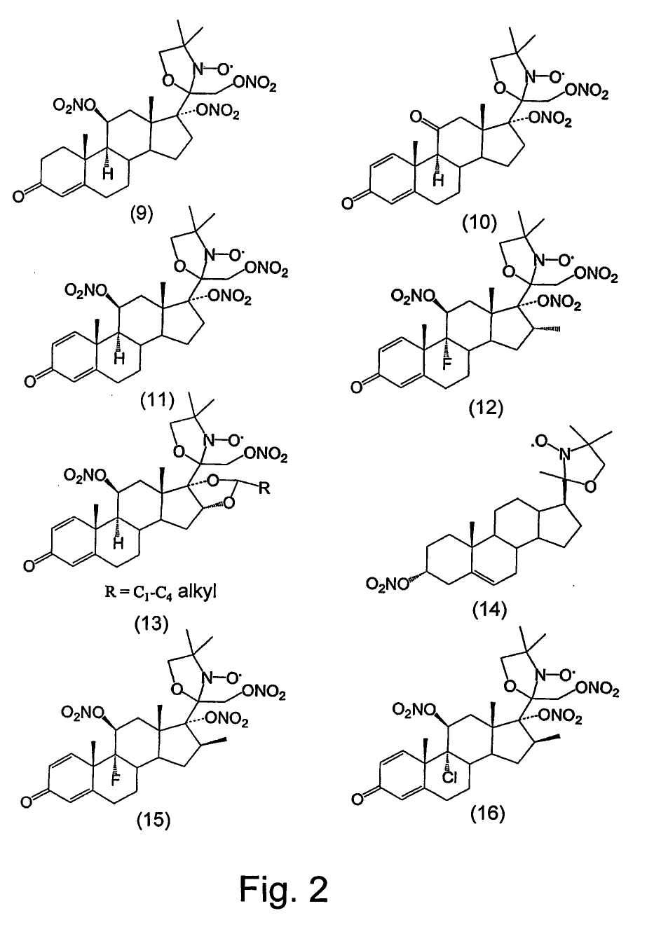

In particular embodiments of formulae IV (IVa-IVd), the multifunctional steroid compounds include compounds 9-23 in FIGS. 2, 3, 4 and 5.

In one embodiment of formulae IV (IVa-IVd), R¹ is —H, —OH, —SNO, —ONO, or —ONO₂, e.g., —SNO or —ONO₂.

In certain embodiments of formulae IV (IVa-IVd), R² is —H, —ONO, —ONO₂, or —SNO, e.g., —H, or —ONO₂.

In one embodiments of formulae IV (IVa-IVd), R³ is —H.

In particular embodiments of formulae IV (IVa-IVd), R⁴ is —H, —Cl or —F, e.g., —H or —F.

In certain embodiments of formulae IV (IVa-IVd), R⁵ is —H, —ONO, —ONO₂, or —SNO. In other embodiments, R⁵ is —H or —ONO₂.

In some embodiments of formulae IV (IVa-IVd), R⁸ is C₁-C₃ alkyl, e.g., methyl or ethyl.

In one embodiment, R⁸ is a 5- or 6-member heteroaryl (e.g. furan, pyrrole, thiazole, oxazole, thiophene, pyridine, imidazole, or pyran

In some embodiments of the formulae IV (IVa-IVd), R⁹ and R¹⁰ are independently, C₁-C₃ alkyl, e.g. methyl or ethyl.

In some embodiments of formulae IV (IVa-IVd), R¹², R¹⁴ and R¹⁵, may be, independently, selected C₁-C₃ alkyl (e.g. methyl, ethyl or butyl) or furan.

In particular embodiments of formulae IV (IVa-IVd), Z is —CH₂—.

In some embodiments of formulae IV (IVa-IVd), X is —O— or —CH₂—, e.g., —O—.

In particular examples of formulae IV (Iva-IVd), where R⁵ includes a substituted N-oxide free radical where the nitrogen of the substituted N-oxide free radical is within a 5- or 6-member ring, the one or more substituted N-oxide free radicals may be, independently, substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical (which are also described by formulae 3a-3b, above).

In other examples of formulae IV (IVa-IVd), where R⁵ includes a substituted N-oxide free radical where the nitrogen of the substituted N-oxide free radical is within a 5- or 6-member ring, the one or more substituted N-oxide free radicals may be, independently, substituted 3-oxazolidinyloxy free radical.

In other examples of formulae IV (IVa-IVd), R² and R⁵ may be, independently, —H, —ONO₂ or —SNO.

In other examples of formulae IV (IVa-IVd), R² and R⁵ may be, independently, —H, —ONO₂ or —SNO.

Compounds of formulae V (Va-Vd) below also are provided.

In one embodiment:

R³ is —H, —OH, or —CH₃;

R⁴ is —H, or halogen (e.g., —F, —I, —Br or —Cl);

R⁵ is —H, ═O, —ONO, —ONO₂, —SNO, —NONOate or a substituted N-oxide free radical; wherein the nitrogen of the N-oxide group in the substituted N-oxide free radical is within a 5- or 6-member ring substituted by one or more independently selected C₁-C₅ alkyl groups (e.g. C₁-C₃, methyl or ethyl);

R⁶ is ═O, —ONO, —ONO₂, —SNO, —NONOate and R^{6A}, if present, is —H, or R⁶ and R^{6A} together form a substituted N-oxide free radical,

wherein the nitrogen of the N-oxide group in the substituted N-oxide free radical is within a 5- or 6-member ring substituted by one or more independently selected C₁-C₅ alkyl groups (e.g. C₁-C₃, methyl or ethyl),
wherein the alkyl substituent group may be further substituted by an NO donor component (e.g. —ONO₂, —SNO), or —OC(O)R¹²,
   wherein R¹² is C₁-C₅ alkyl (e.g. C₁-C₃, methyl or ethyl), or 5- or 6-member heteroaryl (e.g. furan, pyrrole, thiazole, oxazole, thiophene, pyridine, imidazole, or pyran); 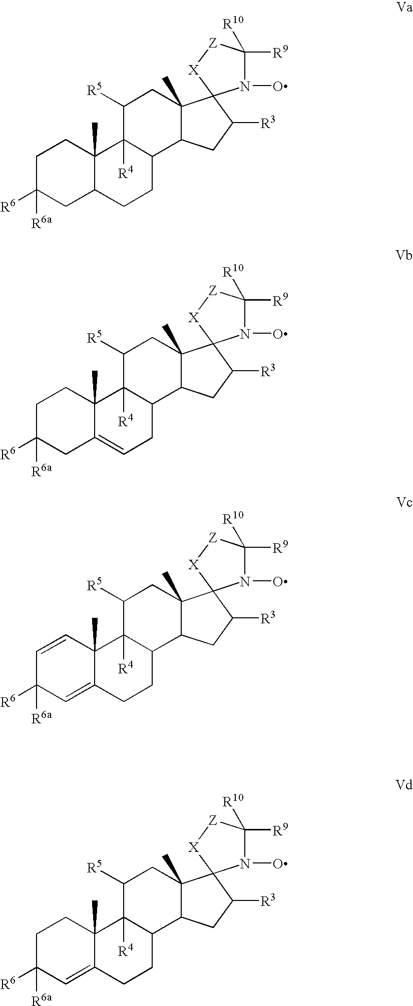
R⁹ and R¹⁰ are independently, linear or branched C₁-C₅ alkyl groups (e.g., C₁-C₃, methyl or ethyl), or substituted linear or branched C₁-C₅ alkyl groups (e.g., C₁-C₃, methyl or ethyl) wherein the alkyl group is independently substituted by —ONO, —ONO₂, —SNO, —NONOate (e.g. —ONO, —NO₂, —SNO) or —OC(O)R¹⁴,
   wherein R¹⁴ is C₁-C₅ allyl (e.g. C₁-C₃), or 5- or 6-member heteroaryl (e.g. furan, pyrrole, thiazole, oxazole, thiophene, pyridine, imidazole, or pyran);

X is —CH₂—, —O— or —S—;

Z is —CH₂— or —CH₂—CH₂—; and

wherein at least one of R⁵, R⁶, R⁹ or R¹⁰ comprises an NO donor.

In certain embodiments of formulae V (Va-Vd),

R³ is —H, —OH, or —CH₃;

R⁴ is —H, —F or —Cl;

R⁵ is —H, ═O, or —NO₂;

R⁶ is ═O, or —NO₂ and R^{6A}, if present, is —H, or R⁶ and R^{6A} together form a substituted N-oxide free radical, e.g. substituted pyrrolinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical; and

R⁹ and R¹⁰ may be, independently, selected C₁-C₂ alkyl;

X is —O— or —CH₂—; and

Z is —CH₂—; and

wherein at least one of R⁵ or R⁶ comprises an NO donor.

In one embodiment of formulae V (Va-Vd),

R⁵ is —H or —NO₂;

R³ and R⁴ are —H;

R⁶ is —ONO₂ or a 5-member substituted N-oxide free radical where the nitrogen of the substituted N-oxide group of the N-oxide free radical is within a 5- or 6-member ring;

R⁹ and R¹⁰ may be, independently, selected C₁-C₂ alkyl;

X is —O— or —CH₂—; and

Z is —CH₂—; and

wherein at least one of R⁵ or R⁶ comprises an NO donor.

In one embodiment of formulae Vb, Vc, and Vd,

R³ is —H or —CH₃;

R⁴ is —H, —F or —Cl;

R⁵ is —ONO₂;

R⁶ is ═O or —ONO₂;

R^{6A}, if present, is —H;

R⁹ and R¹⁰ may be, independently, selected C₁-C₂ alkyl;

X is —O— and

Z is —CH₂—.

In other examples of formulae Vb, Vc, and Vd,

R³ is —CH₃;

R⁴ is —F or —Cl;

R⁵ is —ONO₂;

R⁶ is ═O or —ONO₂;

R^{6A}, if present, is —H;

R⁹ and R¹⁰ may be, independently, selected C₁-C₂ alkyl;

X is —O—; and

Z is —CH₂—.

In one embodiments of formulae V (Va-Vd), R³ is —H.

In particular embodiments of formulae V (Va-Vd), R⁴ is —H, —Cl or —F, e.g., —H or —F.

In certain embodiments of formulae V (Va-Vd), R⁵ is —H, —ONO, —ONO₂, or —SNO. In other embodiments, R⁵ is —H, or —ONO₂.

In some embodiments of formulae V (Va-Vd), R⁶ is ═O, —ONO₂, —SNO, or a substituted N-oxide free radical, e.g., ═O, or —ONO₂.

In some embodiments of formulae V (Va-Vd), R⁸ is C₁-C₃ alkyl, e.g., methyl or ethyl.

In one embodiment, R⁸ is a 5- or 6-member heteroaryl (e.g. fran, pyrrole, thiazole, oxazole, thiophene, pyridine, imidazole, or pyran

In some embodiments of the formulae V (Va-Vd), R⁹ and R¹⁰ are independently, C₁-C₃ alkyl, e.g. methyl or ethyl.

In some embodiments of formulae V (Va-Vd), R¹¹ may be, independently, selected C₁-C₃ alkyl (e.g. methyl, ethyl or butyl) and halogen may be —F.

In some embodiments of formulae V (Va-Vd), R¹², R¹⁴ and R¹⁵, may be, independently, selected C₁-C₃ alkyl (e.g. methyl, ethyl or butyl) or furan.

In particular embodiments of formulae V (Va-Vd), Z is —CH₂—.

In some embodiments of formulae V (Va-Vd), X is —O—, —CH₂—, or —S—, e.g., —O— or —CH₂—.

In particular examples of formulae V (Va-Vd), where R⁵ or R⁶ include a substituted N-oxide free radical where the nitrogen of the substituted N-oxide free radical is within a 5- or 6-member ring, the one or more substituted N-oxide free radicals may be, independently, substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical or substituted thiazinyloxy N-oxide free radical (which are also described by formulae 3a-3b, above).

In other examples of formulae V (Va-Vd), where R⁵ or R⁶ include a substituted N-oxide free radical where the nitrogen of the substituted N-oxide free radical is within a 5- or 6-member ring, the one or more substituted N-oxide free radicals may be, independently, substituted 3-oxazolidinyloxy free radical.

In other examples of formulae V (Va-Vd), R⁵ and R⁶ may be, independently, —H, —ONO₂ or —SNO.

In one embodiment, provided are compounds of formulae VI (VIa-VId), below:

A preferred embodiment of the invention comprises two steroid structures linked by a PEG linker. In certain embodiments are provided compounds of formulae VI below (VIa-VId) where:

R² is —H, —ONO, —ONO₂, —SNO, —OH, —CH₃, —NONOate, or —OC(O)R⁸,
wherein R⁸ is C₁-C₅ alkyl (e.g. C₁-C₃), or 5- or 6-member heteroaryl (e.g. furan, pyrrole, thiazole, oxazole, thiophene, pyridine, imidazole, or pyran);

R³ is —H, —OH, or —CH₃; or
R² and R³ together form a heterocyclic ring;

R⁴ is —H or halogen (e.g., —F, —I, —Br or —Cl);

R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups (e.g. C₁-C₃, methyl or ethyl);

R⁹ and R¹⁰ are independently, linear or branched C₁-C₅ alkyl groups (e.g., C₁-C₃, methyl or ethyl), or substituted linear or branched C₁-C₅ alkyl groups (e.g. C₁-C₃, methyl or ethyl) wherein the alkyl group is independently substituted by —ONO, —ONO₂, —SNO, —NONOate (e.g. —ONO, —ONO₂, —SNO) or —OC(O)R¹⁴, 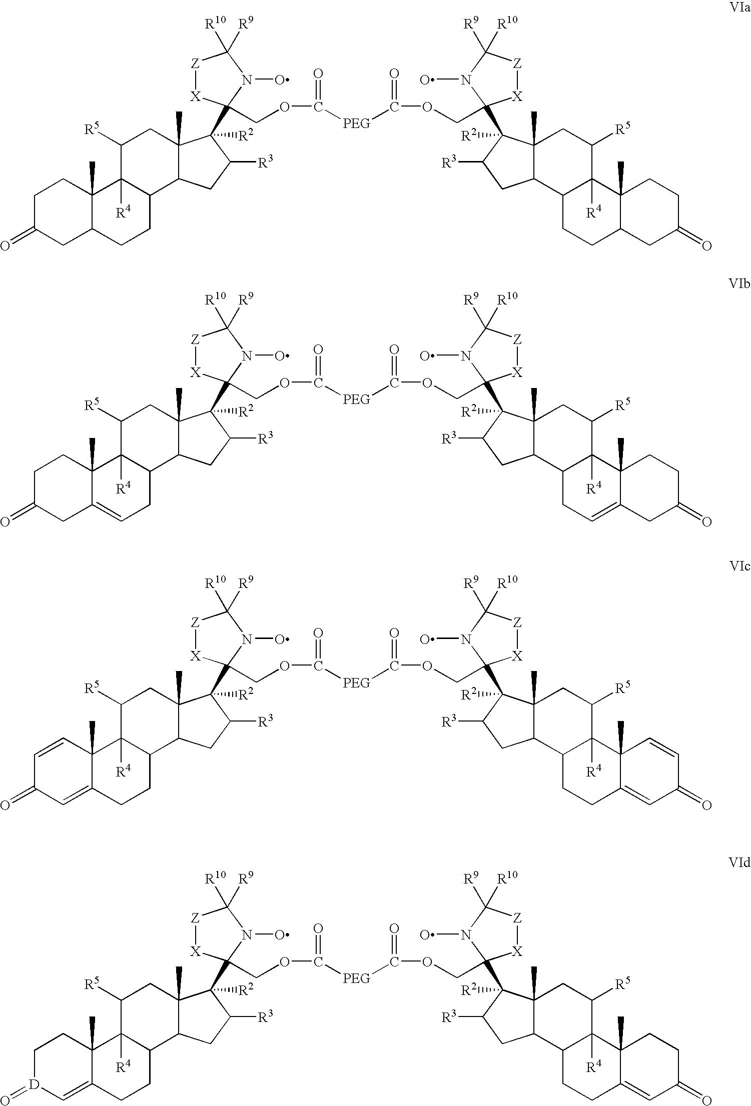
wherein R¹⁴ is C₁-C₅ alkyl (e.g., C₁-C₃, methyl or ethyl), or 5- or 6-member heteroaryl (e.g. furan, pyrrole, thiazole, oxazole, thiophene, pyridine, imidazole, or pyran);

X is —CH₂—, —O— or —S—;

Z is —CH₂— or —CH₂—CH₂—; and

wherein at least one of R², R⁵, R⁹ or R¹⁰ comprises at least one NO donor.

In certain embodiments of formulae VI (VIa-VId),

R² is —H or —ONO₂;

R³ is —H, —OH, or —CH₃;

R⁴ is —H, —F or —Cl;

R⁵ is —H, ═O, or ONO₂;

R⁹ and R¹⁰ may be, independently, C₁-C₂ alkyl (e.g., methyl or ethyl);

X is —O— or —CH₂—; and

Z is —CH₂—; and

wherein at least one of R² or R⁵ comprises at least one NO donor.

In one embodiment of formulae VI (VIa-VId),

R² and R⁵ are —H or —ONO₂;

R³ and R⁴ are —H;

R⁹ and R¹⁰ may be, independently, C₁-C₂ alkyl (e.g., methyl or ethyl);

X is —O— or —CH₂—; and

Z is —CH₂—; and

wherein at least one of R² or R⁵ comprises at least one NO donor.

In one embodiment of formulae VIb, VIc, and VId,

R² is —H or —ONO₂;

R³ is —H or —CH₃;

R⁴ is —H, —F or —Cl;

R⁵ is —H, ═O or —ONO₂;

R⁹ and R¹⁰ may be, independently, C₁-C₂ alkyl (e.g., methyl or ethyl);

X is —O—; and

Z is —CH₂—.

In other examples of formulae VIb, VIc, and VId,

R² and R⁵ are —ONO₂;

R³ is —CH₃;

R⁴ is —F or —Cl;

R⁹ and R¹⁰ may be, independently, C₁-C₂ alkyl (e.g., methyl or ethyl);

X is —O—; and

Z is —CH₂—.

In certain other embodiments of formulae VI (VIa-VId), R² is —H, —ONO₂, or —SNO.

In some embodiments of formulae VI (VIa-VId), R³ is —H.

In particular embodiments of formulae VI (VIa-VId), R⁴ is —H, —Cl or —F, e.g., —H or —F.

In certain embodiments of formulae VI (VIa-VId), R⁵ is —H, —ONO₂, or —SNO, e.g., —H, or —ONO₂.

In some embodiments of formulae VI (VIa-VId), R¹ and R¹⁴ may be, independently, C₁-C₃ alkyl (e.g., methyl or ethyl) or furan.

In some embodiments of formulae VI (VIa-VId), R⁹ and R¹⁰ are independently, C₁-C₃ alkyl (e.g., methyl or ethyl).

In particular embodiments of formulae VI (VIa-VId), Z is —CH₂—.

In some embodiments of formulae VI (VIa-VId), X is or —CH₂—.

In particular examples of formulae VI (VIa-VId), where R⁵ includes a substituted N-oxide free radical where the nitrogen of the N-oxide group of the substituted N-oxide free radical is within a 5- or 6-member ring, the N-xodie free radical is, independently, a substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazohdinyloxy N-oxide free radical or substituted thiazinyloxy N-oxide free radical (which are also described by formulae 3a-3b, above).

In other examples of formulae VI (VIa-VId), where R⁵ includes a substituted N-oxide free radical where the nitrogen of the N-oxide group of the substituted N-oxide free radical is within a 5- or 6-member ring, the one or more N-oxide free radical is, independently, substituted oxazolidinyloxy free radical.

In other examples of formulae VI (VIa-VId), R² and R⁵ may be, independently, —H, —ONO₂ or —SNO.

In particular embodiments of formulae VI (VIa-VId), the multifunctional steroid compounds include the compounds in FIGS. 6 and 7.

In certain examples of multifunctional steroid compounds of formulae I-V where R¹ is —OCO-PEG (e.g., PEG molecular weight from about 100 to about 4000 daltons), or R⁷ includes —OCO-PEG, a dimer of the compound may be formed. For example, formulae VI can be viewed as a dimer of formulae IV, in which the two monomers of formulae IV are linked by R¹. Exemplary dimer multifunctional steroid compounds are shown in FIGS. 6 and 7.

In formulae VI, the C═O on either side of the PEG moiety are functional groups incorporated onto the PEG moiety.

In particular embodiments of Formula I-VI, the one or more substituted N-oxide free radicals may be independently selected from the substituted 5- or 6-member rings as shown below by the general formula (structures 3a-3b):

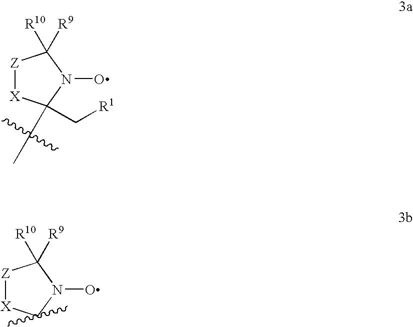

wherein X is —CH₂—, or —S—;

Z is —CH₂— or —CH₂—CH₂—; and

R¹ is —H, —OH, —OCOCH₂-PEG (e.g., PEG molecular weight from about 100 to about 4000 daltons); linear or branched C₁-C₂ alkyl, linear or branched C₁-C₂ alkyl substituted by —ONO, —ONO₂, —SNO, or —NONOate, (e.g., —ONO, —ONO₂, or —SNO) or —OC(O)R¹⁵,
wherein R¹⁵ is C₁-C₅ alkyl (e.g. C₁-C₃), or 5- or 6-member heteroaryl (e.g. furan, pyrrole, thiazole, oxazole, thiophene, pyridine, imidazole, or pyran); and

R⁹ and R¹⁰ are independently, linear or branched C₁-C₅ alkyl groups (e.g., C₁-C₃), or substituted linear or branched C₁-C₅ alkyl groups (e.g., C₁-C₃),
wherein the alkyl group is independently substituted by —ONO, —ONO₂, —SNO, —NONOate (e.g. —ONO, —ONO₂, —SNO) or —OC(O)R¹⁴,
   wherein R¹⁴ is C₁-C₅ alkyl (e.g. C₁-C₃), or 5- or 6-member heteroaryl (e.g. furan, pyrrole, thiazole, oxazole, thiophene, pyridine, imidazole, or pyran).

Where more than one nitroxide radical is present, the substituents alpha to the nitroxide may be independently selected.

As indicated by the wavy line, the substituted nitroxide may be attached to the steroid component by a carbon-to-carbon bond (3a) (see compounds 1-4) or by sharing of a carbon atom as shown in 3b, as, for example in compounds 5-8.

Synthesis of Multifunctional Steroid Compounds

Multifunctional steroid compounds may be synthesized as described herein using methods available in the art and standard techniques in organic chemistry, as described, for example, in March's Advanced Organic Chemistry: Reactions, Mechanisms, and Structure, 5th Edition (2000) M. B. Smith & J. March, John Wiley & Sons, New York, N.Y.; Organic Chemistry 6th Ed. (1992) R. Morrison & R Boyd, Benjamin Cummings, San Francisco; Swartz et al. (1978) Drugs 16:238-255.

Steroids comprising a non-aromatic keto moiety can be converted using a 2-amino-2-methylpropanol into a 2,2,5,5-tetra-substituted amide (pyrazoline), which can be oxidized to yield the free radical SOD mimic component of the multifunctional steroid compound. This amine functionalization of the alcohol is known in the art as a method for the protection of keto moieties, which, in this instance, is used to functionalize commercially available steroids with a desired SOD mimic component. The process of protecting and converting the keto moiety does not alter the other steroid functional groups. Optionally, orthogonal protecting strategies may also be used. Additionally, if desired, more than one keto moiety can be converted into SOD mimic components, which may be the same or different.

Following functionalization of the steroid with one or more SOD mimic components, any one or more of the hydroxyl functional groups present on the steroid can be converted via nitration or nitrosation into an NO donor component.

Additionally, the steroid so modified can be functionalized with, for example, polyethylene glycol (PEG) at position 21. If the PEG is mono-activated PEG then the multifunctional steroid compound will be PEG functionalized. If di-functionalized PEG is used, dimers of the multifunctional steroid compounds may be formed with PEG serving as a linker attached at position 21 of each steroid component. See for example, formulae VI, Examples 1 and 2 and FIGS. 6-7.

In one embodiment, steroid compounds have at least one —OH and at least one keto functional group which can be used to functionalize the compound with NO donor and SOD mimic components, respectively. The keto moieties may optionally be converted to sulphoxides and then protected by the mechanism to obtain thiazoline moieties. To obtain a 6-member ring containing an N-oxide free radical, a substituted 2-amino-butanol is used. Similarly, the substitution of the 2-aminoalcohol will alter the substitution of the ring containing the N-oxide free radical. For example, the use of 2-amino-2-hydroxymethylpropanol can be used to obtain a hydroxyl group on the ring containing the N-oxide free radical. If desired the hydroxyl group can be further functionalized as discussed above. These and similar modifications are well within the skill of those in the art.

Examples of multifunctional steroid compounds and their synthesis are comprised in FIGS. 1-7, in Examples 1-13, as well as in the following schemes.

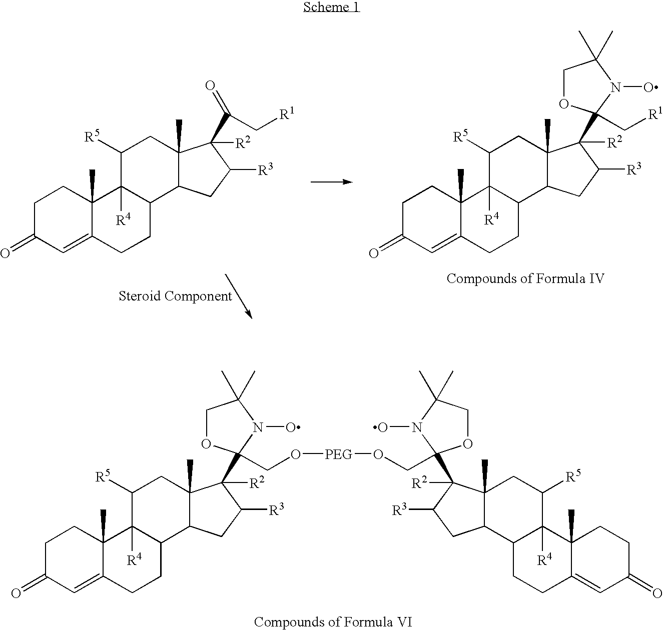

A mixture of one equivalent of the steroid, 5-10 equivalents of 2-amino 2-methylpropanol (higher ratio is used to facilitate dissolution of some steroids in benzene) and a catalytic amount of paratoluenesulfonic acid in benzene is refluxed for 24-48 hours using a Dean-Stark apparatus to remove water. After cooling to room temperature, the reaction mixture is washed with 5% sodium hydroxide solution, water and brine. The solvent is evaporated in a rotary evaporator and the crude product purified by column chromatography and recrystallized in the appropriate solvent (usually ether-hexane mixture). The product is dissolved in acetonitrile (DMSO may in certain cases be added to facilitate dissolution) and hydrogen peroxide is added with sodium tungstate/EDTA and worked up for the specific examples. The crude product is purified by chromatography and dissolved in dry tetrahydrofuran and cooled on an ice path. A stream of nitrogen tetroxide is passed through until the solution turns purple. The reaction flask is capped and the mixture stirred overnight at room temperature. The tetrahydrofuran is evaporated and the residue is dissolved in chloroform and washed repeatedly with 10% sodium bicarbonate solution, water and brine. The organic phase is evaporated and the product is purified by chromatography.

The compounds as described herein may be identified and characterized according to techniques known in the art, including nuclear magnetic resonance (NMR), mass spectrometry (MS) and electron paramagnetic resonance/electron spin resonance (EPR/ESR) as described in the data.

Respiratory Disorders

The present invention provides the compounds of the present invention for use in therapy.

The present invention further provides use of the compounds of the present invention in the manufacture of a medicament for the treatment of respiratory disorders.

Multifunctional steroid compounds are useful in treating a variety of respiratory disorders. Respiratory disorders include asthma, chronic bronchitis, bronchiectasis and emphysema, Chronic Obstructive Pulmonary Diseases (COPDs) or Chronic Obstructive Airway Disease (COADs). Further discussion of the use of treatments can be found in Skorodin (1993) Arch. Intern. Med 153:814.

COPDs are often characterized as being accompanied by chronic or recurrent obstruction to air flow within the lung. Increased resistance to air flow may result from narrowing or restriction of an airway at any level, including partial or complete obstruction from the trachea and larger bronchi to the terminal and respiratory bronchioles.

Another major class of pulmonary or respiratory diseases are often referred to as restrictive diseases, which maybe characterized by reduced expansion of lung parenchyma, with a reduced total lung capacity. Many pathologic conditions associated with respiratory disorders have both obstructive and restrictive components (Cotran et al., “Robbins Pathologic Basis of Disease” 4th Ed. 1989, W.B. Saunders Co., Philadelphia, Pa., pp 755-797).

Asthma may be characterized as a obstructive respiratory disorder characterized by increased responsiveness of the airway to various stimuli, which may potentiate spasmic constriction of the bronchial airways. Asthma can occur secondarily to a variety of stimuli (Cotran et al., “Robbins Pathologic Basis of Disease” 4th Ed. 1989, W.B. Saunders Co., Philadelphia, Pa., pp 755-797). Chronic asthma can also be considered to be predominantly an inflammatory disease with associated bronchospasm. The degree of reactivity and narrowing of the bronchi in response to stimuli is greater in asthmatics than in normal individuals. Persistent inflammation may be responsible for the bronchial hyperreactivity or airway hyperresponsiveness (AHR). Mucosal edema, mucus plugging and hypersecretion can be also present; and pulnonary parenchyma can be common. Asthmatics manifesting such imbalance usually have hyperactive bronchi and even without symptoms, bronchoconstriction may be present. Overt asthma attacks may occur when such individuals are subjected to various stresses, such as viral respiratory infection, exercise, emotional upset, nonspecific factors (e.g., changes in barometric pressure or temperature), inhalation of cold air or irritants (e.g., gasoline fumes, fresh paint and noxious odors, or cigarette smoke), exposure to specific allergens, and ingestion of aspirin or sulfites in sensitive individuals. Asthma is often characterized as “extrinsic” or “allergic”, where the asthmatic episode is precipitated by allergens (e.g. most commonly airborne pollens and molds, house dust, animal danders) or “nonallergic” or “intrinsic”, where symptomatic episodes seem to be triggered by non-allergenic factors (e.g. infection, irritants, emotional factors). In some individuals both allergenic and non-allergenic factors may be significant.

The compounds described herein can be used in the treatment of intrinsic and extrinsic asthma. They are especially applicable to the treatment of allergic or atopic (e.g. IgE-mediated) asthma or non-atopic asthma, as well as exercise induced asthma, occupational asthma, asthma induced following bacterial infection or drug, e.g. aspirin, ingestion and other non-allergic asthmas. The multifunctional steroid compounds may also be used in the treatment and/or prophylaxis of respiratory conditions such as chronic obstructive pulmonary or airways disease (COPD or COAD), chronic bronchitis, emphysema, respiratory distress syndrome (in child or adult), pneumonia, bronchial hyperreactivity, bronchiectasis, and airway hyperresponsiveness (AHR).

Asthma is often categorized as atopic (allergic), nonreaginic (where precipitating factor is a respiratory infection), pharmacologic (e.g. aspirin-sensitive or other drug sensitivity), occupational (e.g. chemical challenge from environmental stimuli), allergic bronchopolumonary aspergillosis (antigen challenge (e.g. spores)) (Cotran et al., “Robbins Pathologic Basis of Disease” 4th Ed. 1989, W.B. Saunders Co., Philadelphia, Pa., pp 755-797). The multifunctional steroid compounds discussed herein may be used in the treatment of each of these conditions or where combinations of factors are responsible for the clinical manifestation of the disorder.

Chronic bronchitis is a condition often associated with prolonged exposure to bronchial irritants and accompanied by mucus hypersecretion and certain structural changes in the bronchi. Usually associated with cigarette smoking, it is characterized clinically by chronic productive cough. Chronic obstructive bronchitis is often characterized as chronic bronchitis associated with extensive alterations of the small airways leading to clinically significant airways obstruction (Cotran et al., “Robbins Pathologic Basis of Disease” 4th Ed. 1989, W.B. Saunders Co., Philadelphia, Pa., pp 755-797).

The present invention is especially applicable in the treatment of respiratory conditions including, but not limited to, the respiratory disorders disclosed herein. As used herein, and as well-understood in the art, “treatment” is an approach for obtaining beneficial or desired results, including clinical results. For purposes of this invention, beneficial or desired clinical results can include one or more, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

Preferred are compounds that are potent and can be administered locally at very low doses, thus avoiding systemic adverse effects. Anti-inflammatory compounds also are preferred that possess cGMP stimulating activity via activation of guanylyl cyclase. Also preferred are compounds with potent antioxidant characteristics. The multifunctional steroid compounds with anti-superoxide activity and NO donor properties can exert a significant impact on the severity, control, and the natural course of respiratory diseases involving oxidative stress and free radical injury. Because of their multi-mechanistic actions, tolerance to their broncho-protective effect can preferably be avoided. The absence of tolerance can avoid the necessity of medium- to high-dose steroid therapy. The development of tolerance is disadvantageous since when administering a composition or drug in repeated dosage or over a period of time, the amount of the composition or the frequency of administration of the drug or composition given to the patient must be increased in order to achieve the same effect as the lower dosage given at an earlier time point in the course of treatment.

Other Disorders

The present invention provides the compounds of the present invention for use in therapy in conditions in which the use of steroids is indicated.

The present invention further provides use of the compounds of the present invention in the manufacture of a medicament for the treatment of conditions in which the use of steroids is indicated.

The multifunctional steroid compounds, and compositions comprising the multifunctional steroid compounds, may be used in methods of treating conditions where treatment with steroids (including designed analogues) is indicated, such as the respiratory disorders discussed above and, in addition, but not limited to, those discussed below. Such conditions include: allergic conditions, arthritis (e.g. rheumatoid or osteo arthritis), autoimmune conditions (e.g. autoimmune destruction of erythrocytes, autoimmune hematologic disorders, systemic lupus erythematosus, graft-vs.-host disease, etc.), cerebral edema, chronic adrenal insufficiency, congenital adrenal hyperplasia, gastrointestinal diseases, hepatic diseases, inflammatory bowel disease, malignancies, multiple sclerosis, neoplastic disease, ocular diseases, ophthalmic disorders, transplantation, including bone marrow and organ transplantation, skin conditions (e.g. psoriasis, contact dermatitis, atopic dermatitis, exfoliative dermatitis, acne, hirsutism, erythema nodosum, inflamed cysts, discoid lupus, bullous diseases, collagen vascular diseases, sarcoidosis, Sweet's disease), renal disease, rheumatic disorders, sarcoidosis, systemic dermatomyositis, cancer, vasculitis, arteritis (e.g., temporal arteritis), and thrombocytopenia The use of steroids for the treatment of the above-listed conditions are known to those of skill in the art (see, for example Goodman & Gillman, supra; Remington: The Science and practice of Pharmacy 20th Ed. (2000) Lippincott Williams and Wilkins, Ed. K. E. Hoover, Merck Index; Sanders et al. Am. J. Respir. Crit. Care. Med., (1995) 151: 1725-33) and the use of the multifunctional steroid compounds described herein in the treatment of these conditions has the benefit of increasing the efficacy of the treatment while decreasing the side effects associated with steroid treatment, and lowering toxcity. The use of the multifunctional steroid compounds described herein may be of particular use in the treatment of allergic conditions, including skin conditions, for example, psoriasis, contact dermatitis, atopic dermatitis; multiple sclerosis; inflammatory bowel disease; and respiratory disorders, as, for example, asthma.

Use of the multifunctional steroid compounds for the treatment of conditions such as psoriasis, inflammatory bowel disease and respiratory disorders is particularly contemplated.

As is known by those of skill in the art, a number of conditions, including some of those listed above, have interacting etiologies and progress over years. Certain conditions, such as diabetes mellitus or asthma, are closely associated with higher incidence of other conditions or complications and treating the root condition (e.g., diabetes, asthma) can elimate the appeance of the associated conditions (e.g., diabetic retinopathy, angina, artherosclerosis, endothelial dysfunction, neuropathy, etc.).

Similarly, many of the conditions for which treatment with steroids is indicated include in their etiology inflammation and the symptoms associated with these conditions can be treated with anti-inflammatory agents, such as the multifunctional steroid compounds described herein (e.g., asthma, artherosclerosis, arthritis, psoriasis, inflammatory bowel disease, etc.).

The vasodilator effect of the multifunctional steroid compounds is useful in numerous conditions, including, but not limited to, asthma, erectile dysfunction, angina, etc.

In addition, as discussed previously, hormonal (sometimes referred to as “sex steroids”) steroids, such as estrogen, progesterone, and testosterone (and designed analogues of these hormones), may be used as the steroid component of the multifunctional steroid compounds and used in the treatment of hormonal conditions, such as conditions associated with fertility, including, menopause, ovarian dysfunction, and testicular dysfunction; or to treat premature baldness. Often, fertility conditions have etiologies including oxidative stress which can also be treated with the multifunctional steroid compounds. Administration of the multifunctional steroid compounds where the steroid component is a hormonal steroid is preferably topical or via injection or suppository (e.g., urethral suppository).

As is discussed herein, the multifunctional steroid compounds are indeed multifunctional, and as discussed above, the skilled practioner will appreciate the use of these compounds in the treatment of a variety of conditions depending on the underlying etiology of the condition and/or the side effects or related conditions associated with the root condition.

Formulations and Dosage

The compounds can be provided in a variety of formulations and dosages. The compounds may be provided in a pharmaceutically acceptable form and/or in a salt form.

In one embodiment, the compounds are provided as non-toxic pharmaceutically acceptable salts. Suitable pharmaceutically acceptable salts of the compounds of this invention include acid addition salts such as those formed with hydrochloric acid, fumaric acid, p-toluenesulphonic acid, maleic acid, succinic acid, acetic acid, citric acid, tartaric acid, carbonic acid or phosphoric acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The pharmaceutically acceptable salts of the present invention may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope solvates of the multifunctional steroid compounds and salts thereof, for example, hydrates.

The multifunctional steroid compounds may have one or more asymmetric centres, and may accordingly exist both as enantiomers and as diastereoisomers. It is to be understood that all such isomers and mixtures thereof are encompassed within the scope of the present invention.

The multifunctional steroid compounds may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, urethral (e.g., urethral suppository) or topical routes of administration (e.g., gel, ointment, cream, aerosol, etc.) and may be formulated, alone or together, in suitable dosage unit formulations containing conventional non-toxic pharmaceutically acceptable carriers, adjuvants, excipients and vehicles appropriate for each route of administration. In addition to the treatment of warm-blooded animals such as mice, rats, horses, cattle, sheep, dogs, cats, monkeys, etc., the compounds of the invention may be effective in humans.

The pharmaceutical compositions for the administration of the multifunctional steroid compounds may conveniently be presented in dosage unit form and may be prepared by any of the methods well known in the art of pharmacy. The pharmaceutical compositions can be, for example, prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired therapeutic effect.

The pharmaceutical compositions containing the multifunctional steroid compound as active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. The pharmaceutical compositions of the invention may also be in the form of oil-in-water emulsions.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that may be employed are water, Ringer's solution and isotonic sodium chloride solution. The multifunctional steroid compounds may also be administered in the form of suppositories for rectal or urethral administration of the drug. In particular embodiments, the multifunctional steroids may be formulated as urethral suppositories, for example, for use in the treatment of fertility conditions, particularly in males, e.g., for the treatment of testicular dysfunction.

According to the invention, multifunctional steroid compounds can be used for manufacturing a composition or medicament, including medicaments suitable for rectal or urethral administration. The invention also relates to methods for manufacturing compositions including multifunctional steroid compounds in a form that is suitable for urethral or rectal adminstration, including suppositories.

For topical use, creams, ointments, jellies, gels, solutions or suspensions, etc., containing the multifunctional steroid compounds may be employed. In certain embodiments, the multifunctional steroid compounds may be formulated for topical administration with polyethylene glycol (PEG). These formulations may optionally comprise additional pharmaceutically acceptable ingredients such as diluents, stabilizers and/or adjuvants. In particular embodiments, the topical formulations are formulated for the treatment of allergic conditions and/or skin conditions including psoriasis, contact dermatitis and atopic dermatitis, among others described herein.

According to the invention, multifunctional steroid compounds can be used for manufacturing a composition or medicament, including medicaments suitable for topical administration. The invention also relates to methods for manufacturing compositions including multifunctional steroid compounds in a form that is suitable for topical administration.

According to the present invention, multifunctional steroid compounds can also be delivered by any of a variety of inhalation devices and methods known in the art, including, for example: U.S. Pat. No. 6,241,969; U.S. Pat. No. 6,060,069; U.S. Pat. No. 6,238,647; U.S. Pat. No. 6,335,316; U.S. Pat. No. 5,364,838; U.S. Pat. No. 5,672,581; WO96/32149; WO95/24183; U.S. Pat. No. 5,654,007; U.S. Pat. No. 5,404,871; U.S. Pat. No. 5,672,581; U.S. Pat. No. 5,743,250; U.S. Pat. No. 5,419,315; U.S. Pat. No. 5,558,085; WO98/33480; U.S. Pat. No. 5,364,833; U.S. Pat. No. 5,320,094; U.S. Pat. No. 5,780,014; U.S. Pat. Nos. 5,658,878; 5,518,998; 5,506,203; U.S. Pat. No. 5,661,130; U.S. Pat. No. 5,655,523; U.S. Pat. No. 5,645,051; U.S. Pat. No. 5,622,166; U.S. Pat. No. 5,577,497; U.S. Pat. No. 5,492,112; U.S. Pat. No. 5,327,883; U.S. Pat. No. 5,277,195; U.S. Pat. App. No. 20010041190; U.S. Pat. App. No. 20020006901; and U.S. Pat. App. No. 20020034477.

Included among the devices which may be used to administer particular examples of the multifunctional steroid compounds are those well-known in the art, such as, metered dose inhalers, liquid nebulizers, dry powder inhalers, sprayers, thermal vaporizers, and the like. Other suitable technology for administration of particular multifunctional steroid compounds includes electrohydrodynamic aerosolizers.

The abbreviations “MMAD” and “MMEAD” are well-known in the art, and stand for “mass median aerodynamic diameter” and “mass median equivalent aerodynamic diameter” respectively. The terms as used in the art are substantially equivalent. The “aerodynamic equivalent” size of a particle is the diameter of a unit density sphere which exhibits the same aerodynamic behavior as the particle, regardless of actual density or shape. MMAD is usually determined using a cascade impactor which measures-the particle size as a function of the aerodynamic behavior of the particle in a high velocity airstream. The median (50%) particle size is obtained from a linear regression analysis of the cumulative distribution data. In one embodiment, the inhalation device delivers small particles, e.g., less than about 10 μm MMAD.

In addition, the inhalation device is preferably practical, in the sense of being easy to use, small enough to carry conveniently, capable of providing multiple doses, and durable. Some specific examples of commercially available inhalation devices are Turbohaler (Astra, Wilmington, Del.), Rotahaler (Glaxo, Research Triangle Park, N.C.), Diskas (Glaxo, Research Triangle Park, N.C.), the Ultravent nebulizer (Mallinckrodt), the Acorn II nebulizer (Marquest Medical Products, Totowa, N.J.) the Ventolin metered dose inhaler (Glaxo, Research Triangle Park, N.C.), or the like. In one embodiment, multifunctional steroid compounds can be delivered by a dry powder inhaler or a sprayer.

As those skilled in the art will recognize, the formulation of multifunctional steroid compounds, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of multifunctional steroid compounds in the aerosol. For example, shorter periods of administration can be used at higher concentrations of multifunctional steroid compounds in the nebulizer solution. Devices such as metered dose inhalers can produce higher aerosol concentrations, and can be operated for shorter periods to deliver the desired amount of multifunctional steroid compounds in some embodiments. Devices such as dry powder inhalers deliver active agent until a given charge of agent is expelled from the device. In this type of inhaler, the amount of multifunctional steroid compounds in a given quantity of the powder determines the dose delivered in a single administration. The formulation of multifunctional steroid compound is selected to yield the desired particle size in the chosen inhalation device.

Dry powder generation typically employs a method such as a scraper blade or an air blast to generate particles from a solid formulation of multifunctional steroid compounds. The particles are generally generated in a container and then transported into the lung of a patient via a carrier air stream. Typically, in current dry powder inhalers, the force for breaking up the solid and air flow is provided solely by the patient's inhalation. One suitable dry powder inhaler is the Turbohaler manufactured by Astra (Wilmington, Del.).

Formulations of multifunctional steroid compounds for administration from a dry powder inhaler may typically include a finely divided dry powder containing multifunctional steroid compounds, but the powder can also include a bulking agent, buffer, carrier, excipient, another additive, or the like. Additives can be included in a dry powder formulation of multifunctional steroid compounds, for example, to dilute the powder as required for delivery from the particular powder inhaler, to facilitate processing of the formulation, to provide advantageous powder properties to the formulation, to facilitate dispersion of the powder from the inhalation device, to stabilize to the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; or the like.

In some embodiments, a spray including multifunctional steroid compounds can be produced by forcing a suspension or solution of a particular multifunctional steroid compound through a nozzle under pressure. The nozzle size and configuration, the applied pressure, and the liquid feed rate can be chosen to achieve the desired output and particle size. An electrospray can be produced by an electric field in connection with a capillary or nozzle feed. Formulations of multifunctional steroid compounds suitable for use with a sprayer can include multifunctional steroid compounds in an aqueous solution at a concentration of about 1 mg to about 20 mg of multifunctional steroid compound per mL of solution. The formulation can include agents such as an excipient, a buffer, an isotonicity agent, a preservative, a surfactant, and/or zinc. Multifunctional steroid compounds can be administered by a nebulizer, such as jet nebulizer or an ultrasonic nebulizer. Typically, in a jet nebulizer, a compressed air source is used to create a high-velocity air jet through an orifice.

Formulations of multifunctional steroid compound suitable for use with a nebulizer, either jet or ultrasonic, typically include multifunctional steroid compound in an aqueous solution. The formulation can include agents such as an excipient, a buffer, an isotonicity agent, a preservative, a surfactant, and/or zinc. The formulation can also include an excipient or agent for stabilization of the multifunctional steroid compound, such as a buffer, a reducing agent, a bulk protein, or a carbohydrate. Bulk proteins, surfactants, carbohydrates and other additives are useful in formulating multifunctional steroid compounds and can be used.

In a metered dose inhaler (MDI), a propellant, multifunctional steroid compound, and any excipients or other additives are contained in a canister as a mixture including a liquefied compressed gas.

The present invention also relates to a pharmaceutical composition including multifunctional steroid compounds suitable for administration by inhalation. According to the invention, multifunctional steroid compounds can be used for manufacturing a composition or medicament, including medicaments suitable for administration by inhalation. The invention also relates to methods for manufacturing compositions including multifunctional steroid compounds in a form that is suitable for administration, including administration by inhalation. For example, a dry powder formulation can be manufactured in several ways, using conventional techniques, such as described in any of the publications mentioned above and incorporated expressly herein by reference, and for example, Baker, et al., U.S. Pat. No. 5,700,904, the entire disclosure of which is incorporated expressly herein by reference. Particles in the size range appropriate for maximal deposition in the lower respiratory tract can be made by micronizing, milling, or the like. And a liquid formulation can be manufactured by dissolving the multifunctional steroid compounds in a suitable solvent, such as water, at an appropriate pH, including buffers or other excipients.

As known by those of skill in the art, the preferred dosage of multifunctional steroid compounds will depend on the age, weight, general health and severity of the disorder of the individual being treated. Dosage may also need to be tailored to the sex of the individual and/or where administered by inhalation, the lung capacity of the individual. Dosage may also be tailored to individuals suffering from more than one disorder or those individuals who have additional conditions which affect lung capacity and the ability to breathe normally, for example, emphysema, bronchitis, pneumonia, respiratory infections, etc. Dosage, and frequency of administration of the multifunctional steroid compound, will also depend on whether the compounds are formulated for treatment of acute episodes of a disorder or for the prophylactic treatment of a disorder. For example, acute episodes of allergic conditions, including allergy-related asthma, dermatitis or other antigen-induced conditions. A skilled practitioner will be able to determine the optimal dose for a particular individual.

As known to those of skill in the art, treatment with steroids, for either or acute or chronic (prophylactic) treatment, attempts to use the lowest possible steroid dosage to achieve the necessary effect, that is, to relieve the symptoms being treated. An advantage of the multifunctional steroid compounds is that they may be administered in a dosage that is less than that of traditional steroids. The dosage of the multifunctional steroid compounds as described herein may be, for example, 5%-10% of the dosage of the steroid from which the steroid component is selected (or e.g., 3%, 5%, 10%, 15%, 20%, 25%, 50% of the dosage of the corresponding steroid component), if the steroid alone were administered. For example, in general, hydrocortisone is administered in dosages ranging from 25 mg to 60 mg per day. The multifunctional steroid compounds may be administered at a dosage of e.g., 50 micrograms per day to 6 mg per day, (e.g. 0.1 mg/day or 1.0 mg/day). Hydrocortisone optionally may be administered twice a day, depending on the factors as described herein. Prednisone, beclamethasone and dexamethasone are usually administered once a day. Belcamethasone and dexamethasone are generally administered in doses ranging from 2.5 mg/day to 60 mg/day. The multifunctional steroid compounds having this steroid component may be administered, for example, at a dosage of 0.1 to 1.0 mg/day, or 1.0 to 10.0 mg/day. The dosage range for steroid compounds may be the same for both oral and parenteral formulations, including formulations for inhalation. In general, adminstration of the multifunctional steroid compound may range, for example, from about 50 micrograms to 60 mg/day, e.g., 0.1 mg/day, 1.0 mg/day or 10 mg/day.

The duration of the dosage will depend upon multiple factors, including the general health and symptoms of the individual and the condition being treated. For example, if an acute condition is being treated, then the duration of treatment may be, for example, from 1 day to 15 days. If a chronic condition is being treated, the duration of treatment may be days, weeks, or months. In the case of inflammation, the lowest effective amount of the steroid is administered to until remission of the condition is attained. In chronic treatments, the lowest possible dosage to achieve abate the symptoms of the condition is administered. For certain acute conditions (e.g., temporal arteritis), high doses of unmodified traditional steroid may be administered for up to 10 days or two weeks, e.g., 80 mg/day. The multifunctional steroid compound may be administered at a lower dosage, e.g. of 0.1 mg/day to 10 mg/day, e.g., 0.1 mg/day, or 1 mg/day, or 10 mg/day. As with traditional steroid treatment known in the art, treatment with multifunctional steroid compounds as described herein may be tapered gradually to lower doses before ending the course of treatment.

Various formulations of the compounds and compositions described herein may be administered according to the variables. In particular, formulations for prophylactic treatment of conditions may be administered, daily, twice daily, thrice daily or four times daily and/or upon the occurrence of symptoms associated with the underlying condition. For respiratory disorders, such symptoms include wheezing, coughing, shortness of breath, tightness or pressure in the chest and the like. For skin conditions, symptoms include redness, swelling, itching, increased skin sensitivity (e.g. to heat, to cold or to sun, etc.), and/or outbreaks of lesions. It is contemplated that individuals who are using a prophylactic formulation may on occasion need to administer doses in response to acute episodes of symptoms. Administration includes any of the methods or routes as described herein.

The compounds as described herein may be administered to an individual in need thereof over a period of time consistent with treatment of the disorder from which the individual suffers. In the case of, for example, pneumonia or other periodic disorders, the treatment may be discontinued when the individual is no longer affected by the disorder or deemed to be no longer in need of the treatment by a skilled practitioner. Examples of such time periods include days, weeks or months. Where the condition is a congenital or chronic disorder such as multiple sclerosis, inflammatory bowel disease, psoriasis, asthma, emphysema, AHR, COPD, fertility condition (e.g., ovarian dysfunction, menopause, testicular dysfunction, etc.) and others, it is envisioned that the treatment with the compounds described herein will be administered for a period of weeks, months, years or decades. The methods as described herein also include the administration of combinations of the compounds as described herein, or combinations of the compounds described herein and other drugs used in the treatment of the disorders described herein or symptoms associated with these disorders.

Drug delivery devices, such as metered inhalation devices, may be used to deliver the compounds of the invention by inhalation.

Also provided are kits for administration of the multifunctional steroid compound or composition comprising at least one multifunctional steroid compound, that may include a dosage amount of at least one multifunctional steroid compound or a composition comprising at least one multifunctional steroid compound as disclosed herein. Kits may further comprise suitable packaging and/or instructions for use of the compound. Kits may also comprise a means for the delivery of the at least one multifunctional steroid compound or compositions comprising at least one multifunctional steroid compound, such as an inhaler, spray dispenser (e.g. nasal spray), syringe for injection or pressure pack for capsules, tables, suppositories, or other device as described herein.

In another aspect of the invention, kits for treating an individual who suffers from or is susceptible to the disorders described herein are provided, comprising a container comprising a dosage amount of an multifunctional steroid compound or composition as disclosed herein, and instructions for use. The container may be any of those known in the art and appropriate for storage and delivery of oral, intravenous, topical, rectal, urethral, or inhaled formulations.

Kits may also be provided that contain sufficient dosages of the multifunctional steroid compound or composition to provide effective treatment for an individual for an extended period, such as a week, 2 weeks, 3, weeks, 4 weeks, 6 weeks or 8 weeks or more.

The invention is further illustrated by the following nonlimiting examples.

All patents, patent applications and publications referred to herein are hereby incorporated herein by reference in their entirety.

### EXAMPLES

### Example 1

### Synthesis of PEG-di-[20-DOXYL-11,17-dinitrato-Prednisolonoate (compound D)

The synthesis is shown in FIG. 6.

Synthesis of intermediate A (FIG. 6): To a mixture of 3.6 g (10 mmol, 1 equivalent) of prednisolone and 2.22 g (25 mmol, 2.5 equivalents) of 2-amino-2-methylpropanol in benzene (100 ml), a catalytic amount of paratoluene sulfonic acid is added and the mixture is refluxed in Dein-Stark apparatus for 48 hr. After cooling, the benzene was evaporated to dryness and the solid residue is washed successively with distilled water and dried in a vacuum desiccator to give intermediate A as a white powder. NMR analysis (DMSOd, 400 MHz) show the expected singlets at 1.15 and 1.2 ppm (3H each) corresponding to the two methyl groups of the DOXYL group and two doublets at 3.25 ppm and 3.41 ppm (1H each) corresponding to the CH₂ of the DOXYL group. The hydroxyl hydrogen at position 17 was shifted, as expected, from 5.19 to 4.2 ppm.

Synthesis of B (FIG. 6): Four equivalents of dicyclohexyl-carbodiimide dissolved in dichloromethane are added dropwise to a vigorously stirred solution of 4 equivalents of A and one equivalent of poly(ethylene glycol) bis(carboxy methyl) ether (ca. 4000) dissolved in dichloromethane and cooled on an ice bath. When addition is complete, the reaction mixture is stirred for 24 hours at room temperature. Upon completion (TLC), the reaction mixture is filtered and the precipitated dicyclohexylurea is discarded. The solvent is evaporated to dryness and the solid residue is dissolved in hot ethanol. Upon cooling, intermediate B crystallizes as fine needles, filtered and washed with ether.

Synthesis of intermediate C (FIG. 6): To one equivalent of B, 10 equivalents of sodium tungstate dihydrate, and 10 equivalents of ethylene diamine tetraacetic acid (EDTA) disodium salt dissolved in 90 ml of distilled water, 10 ml of 30% hydrogen peroxide is added and the mixture is stirred in the dark at room temperature for one week. Water is removed by means of lyophilization and the residue is dissolved in ethanol. The precipitated salts are discarded by filtration. Ethanol is evaporated under reduced pressure and the crude residue is dissolved in a hot mixture of ethanol:ether (1:1) and cooled. The precipitated intermediate C is filtered, dried and collected as a pale yellow powder.

Synthesis of compound D (FIG. 6): Intermediate C is dissolved in dry tetrahydrofuran and the solution is cooled and saturated with nitrogen dioxide gas. The reaction flask is capped and stirred at room temperature overnight. The flask is first cooled, carefully opened, and the solvent is evaporated to dryness. The residue is triturated with ether, filtered and dried to give PEG-di-[20-DOXYL-11,17-dinitrato-prednisolonoate (D in FIG. 6) as a pale green solid.

The yield of all above reactions exceeded 90% and the structures and purity of all intermediates, as well as of the final product PEG-di-[20-DOXYL-11,17-dinitrato-prednisolonoate, compound D, were verified by NMR, MS and elemental analysis.

### Example 2

### Synthesis of PEG-di-[20-DOXYL-11,17-dinitrato-dexamethasonoate (compound H)

FIG. 7 illustrates the synthetic pathway for synthesis of compound H.

Synthesis of intermediate E (FIG. 7): To a mixture of 3.92 g (10 mmol, 1 equivalent) of dexamethasone and 2.22 g (25 mmol, 2.5 equivalents) of 2-amino-2-methylpropanol in benzene (100 ml), a catalytic amount of paratoluene sulfonic acid is added and the mixture is refluxed in a dean-stark apparatus for 48 hr. After cooling, the benzene was evaporated to dryness and the solid residue washed successively with distilled water and dried in a vacuum desiccator to give E as a white solid. NMR analysis (DMSOd₆, 400 MHz) show new singlets at 1.10 and 1.19 ppm (3H each) corresponding to the two methyl groups of the DOXYL group and two doublets appearing at 3.18 ppm and 3.43 ppm (1H each) corresponding to the CH₂ of the DOXYL group. The hydroxyl hydrogen at the position 17 was shifted from 4.97 to 4.15 ppm and the hydroxyl hydrogen at position 21 was shifted from 4.7 to 4.08 ppm.

Synthesis of intermediate F (FIG. 7): Four equivalents of dicyclohexyl-carbodiimide dissolved in dichloromethane are added dropwise to a vigorously stirred solution of 4 equivalents of E and one equivalent of poly(ethylene glycol) bis(carboxy methyl) ether (ca. 4000) dissolved in dicloromethane and cooled on an ice bath. When addition is complete, the reaction mixture is stirred for further 24 hours at room temperature. The reaction mixture is then filtered and the precipitated dicyclohexylurea is discarded. The solvent is evaporated to dryness and the solid residue is dissolved in hot ethanol. Product F precipitated as a white powder upon cooling, filtered and washed with ether.

Synthesis of intermediate G (FIG. 7): To 1 equivalent of F6, 10 equivalents of sodium tungstate, and 10 equivalents of ethylene diamine tetraacitic aid (EDTA) disodium salt dissolved in 90 ml of distilled water, 10 ml of 30% hydrogen peroxide is added and the mixture is stirred in the dark at room temperature for one week. Water is removed by means of lyophilization and the residue is dissolved in ethanol and the precipitated salts removed by filtration. The solvent was evaporated and the crude product was dissolved in hot mixture of ethanol:ether (1:1) and cooled. The precipitate was filtered, dried and collected as a pale yellow powder.

Synthesis of compound H (FIG. 7): Product G is dissolved in dry tetrahydrofuran and the solution is cooled and saturated with nitrogen dioxide gas. The reaction flask is capped and stirred at room temperature overnight. The flask is carefully opened and the solvent is evaporated to dryness. The residue is triturated with ether, filtered and dried to give the title compound PEG-di-[20-DOXYL-11,17-dinitrato-dexamethasonoate (H) as a highly pure pale green solid in 94% yield.

The yield of all above reactions exceeded 90% and the structures and purity of all intermediates, as well as of the final product PEG-di-[20-DOXYL-11,17-dinitrato-dexamethasonoate, were verified by NMR, MS and elemental analysis.

### Examples 3 and 4

### Synthesis of 20-DOXYL-3α-nitrato-5-pregnenoate (2) and 20-DOXYL-3β-nitrato-5-pregnenoate (1)

These compounds were synthesized according to the following sequence:

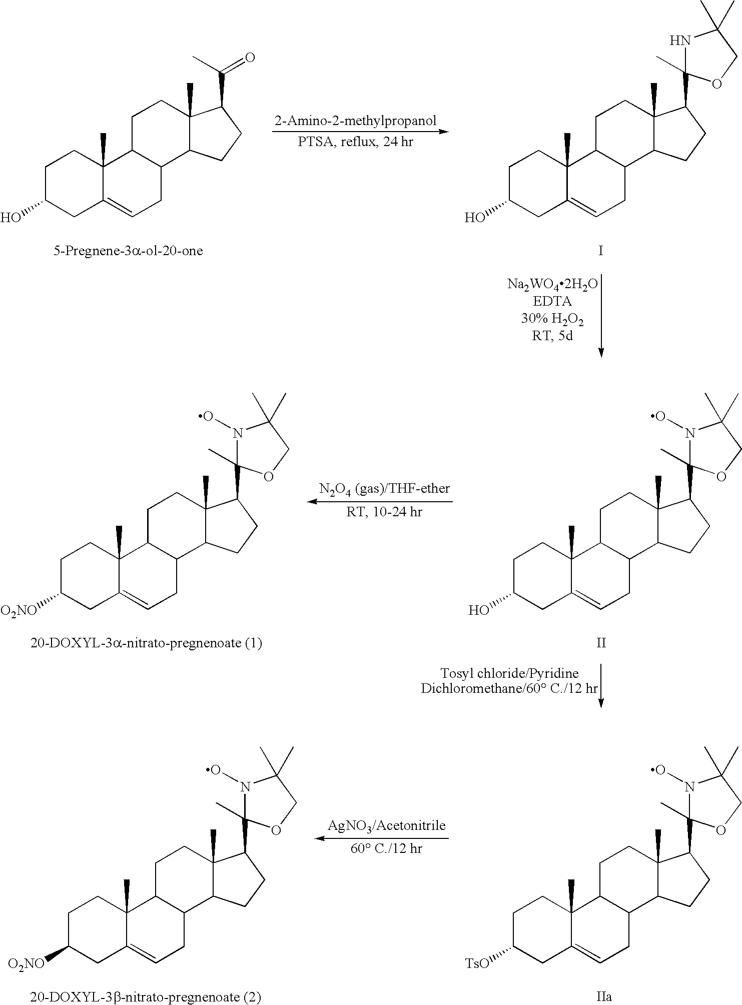

Synthesis of intermediate I: To a mixture of 3.16 g (10 mmol, 1 equivalent) of 5-pregnene-3α-ol-20-one (pregnenolone) and 2.22 g (25 mmol, 2.5 equivalents) of 2-amino-2-methylpropanol in benzene (100 ml), a catalytic amount of paratoluene sulfonic acid (PTSA) is added and the mixture is refluxed in Dein-Stark apparatus until starting material has disappeared (16-24 hr, TLC). After cooling, the benzene was evaporated to dryness and the solid residue is washed successively with distilled water and dried in a vacuum desiccator to give intermediate I as a white powder in 92% yield, which was used for the next step without further purification.

Synthesis of intermediate II: To one equivalent of intermediate I dissolved in 50 ml of methanol, 10 equivalents of sodium tungstate (NaTg) dihydrate, and 10 equivalents of ethylene diamine tetraacetic acid (EDTA) disodium salt dissolved in 40 ml of distilled water, 10 ml of 30% hydrogen peroxide is added in three portions and the mixture is stirred in the dark at room temperature for one week (the reaction is usually complete within 4-6 days). Water is removed by means of lyophilization and the residue is dissolved in minimal amount of chloroform. The precipitated salts are discarded by filtration and the solvent applied to a silica gel column and eluted with petroleum ether—ethyl acetate (4.5:0.5) to furnish a pure, pale-yellow powder in 84% yield.

Synthesis of intermediate IIa: To one equivalent of intermediate II dissolved in 50 ml of dichloromethanol and 3 ml of dry pyridine, 3 mol equivalents of p-toluene sulfonyl chloride in 50 ml of dichloromethane were added dropwise at room temperature. Once addition is complete, the mixture was stirred at 60° C. for 12 hr, or until the reaction is complete (TLC). The mixture is cooled and washed twice with water, twice with 1 N NaOH and once with saturated NaCl solution and dried over magnesium sulfate. The solvent was evaporated to dryness and the residue was used as such for the next step without further purification.

Synthesis of compound 1: Intermediate II is dissolved in dry tetrahydrofuran and the solution is cooled and saturated with nitrogen dioxide gas. The reaction flask is capped and stirred at room temperature overnight. The flask is first cooled, carefully opened, and the solvent is evaporated to dryness. The residue is dissolved in minimal amount of ether and applied to a silica gel column and eluted with hexane to furnish pure 20-DOXYL-3α-nitrato-5-pregnenoate (1) in 98% yield.

Synthesis of compound 2: Intermediate IIa (one equivalent) is dissolved in acetonitrile (if not freely soluble, small amount of DMSO is added) and 2.5 equivalents of silver nitrate are added in three portions at room temperature. The mixture is then heated at 60° C. for 8-12 hr, or until reaction is completed (TLC). The mixture is cooled and the precipitate filtered and discarded. The solvent is evaporated and the residue is dissolved in an ether-hexane mixture (80:20) and filtered again. The solvent is washed twice with water, once with 1 N NaOH, and once with saturated NaCl solution, dried over magnesium sulfate and condensed to ca., 10-15 ml. This residue was applied to a silica gel column and eluted with hexane to furnish pure 20-DOXYL-3β-nitrato-5-pregnenoate 2 in 78% yield (from intermediate II).

### Examples 5 and 6

### Synthesis of 20-DOXYL-3α-nitrato-5-pregnenoate (3) and 20-DOXYL-3β-nitrato-5-pregnenoate (4)

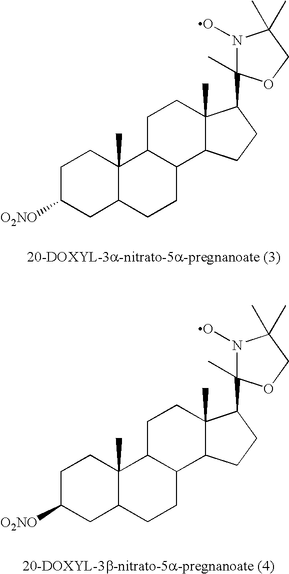

20-DOXYL-3α-nitrato-5α-pregnanoate 3 was synthesized in a similar overall yield and purity essentially as compound 1 above, except that the starting material was 5α-pregnene-3α-ol-20-one. The 3β-nitrato derivative 4 was prepared from the 5α-derivative utilizing the same synthetic pathway described for the conversion of II to 2 above (i.e., tosylation and nitration with conversion of configuration with silver nitrate) in a 73% yield.

### Examples 7 and 8

### Synthesis of 17-DOXYL-cis-androsteronoate-3β-nitrate (5) and 17-DOXYL-trans-androsteronoate-3α-nitrate (6)

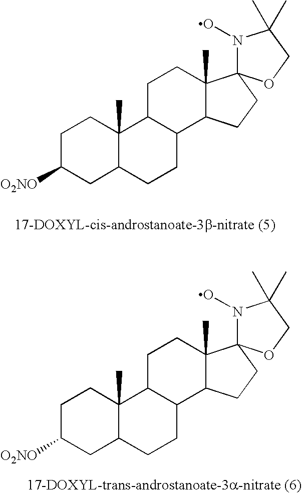

These compounds were synthesized in a similar overall yield and purity according to a sequence that is essentially the same as for compound 1 above with the starting (commercially available) materials cis-androsterone and trans-androsterone.

### Examples 9 and 10

### Synthesis of 3-DOXYL-5α-androstanoate-17β-nitrate (7) and 3-DOXYL-5α-androstanoate-17α-nitrate (8)

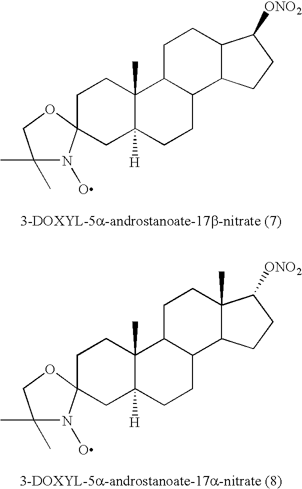

3-DOXYL-5α-androstanoate-17β-nitrate 7 was synthesized in a similar overall yield and purity essentially as compound 1 above starting from commercially available 5α-androstan-17β-ol-3-one (4,5-dihydrotestosterone). The 17α-nitrato derivative 8 was prepared from the 17β-ol-3-one derivative utilizing the same synthetic pathway described for the conversion of II to 2 above (i.e., tosylation and conversion of configuration via nitration with silver nitrate) in a 84% yield.

### Example 11

### Synthesis of 11,17,21-trinitrato-16-DOXYL dexamethasone (12)

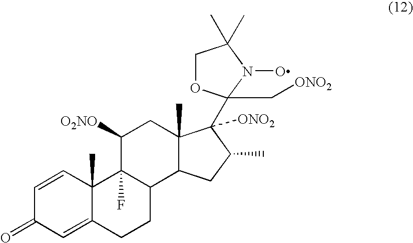

The detailed synthesis of compound 12 (11,17,21-trinitrato-16-DOXYL-dexamethasone) was carried out as described in the following scheme:

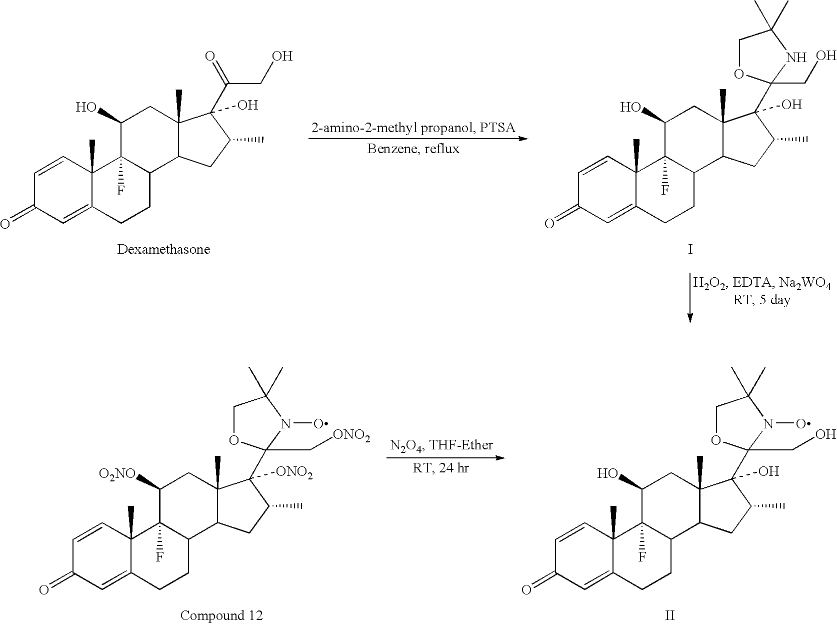

To a mixture of 3.92 g (10 mmol, 1 equivalent) of dexamethasone and 2.22 g (25 mmol, 2.5 equivalents) of 2-amino-2-methylpropanol in benzene (100 ml), a catalytic amount of paratoluene sulfonic acid (PTSA) was added and the mixture was refluxed in Dein-Stark apparatus until staring material has disappeared (48-72 hr, TLC). After cooling, the benzene was evaporated to dryness and the solid residue was washed successively with distilled water and dried in a vacuum diseccator to give intermediate I as a white powder in 92% yield, which was used for the next step without further purification.

Synthesis of intermediate II: To one equivalent of the intermediate dissolved in 50 ml of methanol, 10 equivalents of sodium tungstate (NaWO₄) dihydrate, and 10 equivalents of ethylene diamine tetraacetic acid (EDTA) disodium salt dissolved in 40 ml of distilled water, 10 ml of 30% hydrogen peroxide was added in three portions and the mixture was stirred in the dark at room temperature for one week (the reaction was usually complete within 4-6 days). Water was removed by means of lyophilization and the residue was dissolved in minimal amount of chloroform. The precipitated salts were discarded by filtration and the solvent applied to a silica gel column and eluted with petroleum ether—ethyl acetate (4.5:0.5) to furnish a pure, pale-yellow powder in 84% yield.

Synthesis of compound 12: Intermediate was dissolved in dry tetrahydrofuran and the solution was cooled and saturated with nitrogen dioxide gas. The reaction flask was capped and stirred at room temperature overnight. The flask was first cooled, carefully opened, and the solvent was evaporated to dryness. The residue was dissolved in minimal amount of ether and applied to a silica gel column and eluted with hexane to furnish pure compound 12 (11,17,21-trinitrato-16-DOXYL-dexamethasone) in 90% yield.

### Example 12

### Synthesis of 11,17,21-trinitrato-16-DOXYL-betamethasone) (15)

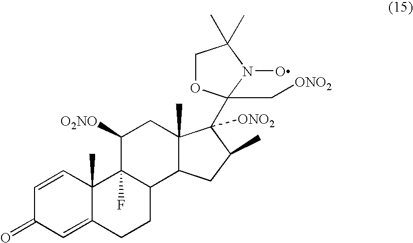

The detailed synthesis of this compound 15 was carried out as described in the following scheme:

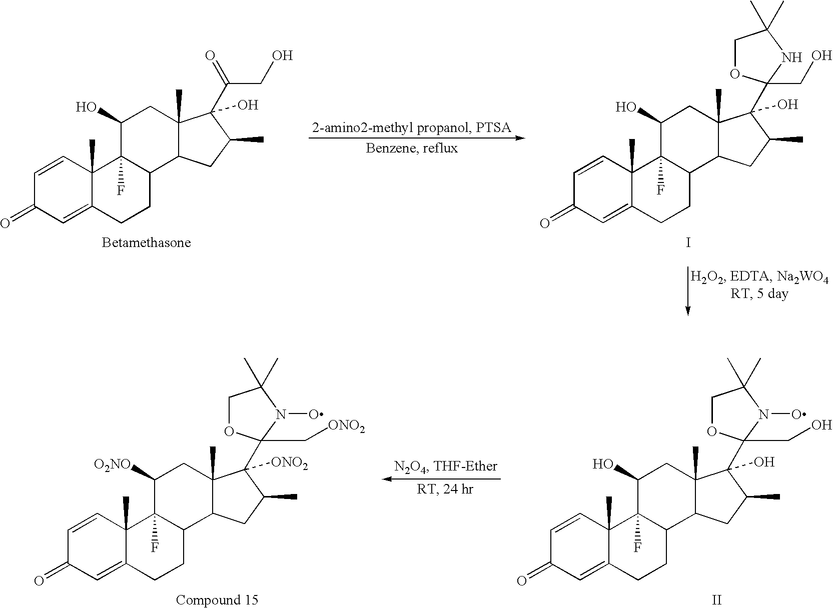

To a mixture of 3.92 g (10 mmol, 1 equivalent) of betamethasone and 2.22 g (25 mmol, 2.5 equivalents) of 2-amino-2-methylpropanol in benzene (100 ml), a catalytic amount of paratoluene sulfonic acid (PTSA) was added and the mixture was refluxed in Dein-Stark apparatus until starting material has disappeared (48-72 hr, TLC). After cooling, the benzene was evaporated to dryness and the solid residue was washed successively with distilled water and dried in a vacuum desiccator to give intermediate I as a white powder in 92% yield, which was used for the next step without further purification.

Synthesis of intermediate: To one equivalent of the intermediate dissolved in 50 ml of methanol, 10 equivalents of sodium tungstate (Na₂WO₄) dihydrate, and 10 equivalents of ethylene diamine tetraacetic acid (EDTA) disodium salt dissolved in 40 ml of distilled water, 10 ml of 30% hydrogen peroxide was added in three portions and the mixture was stirred in the dark at room temperature for one week (the reaction was usually complete within 4-6 days). Water was removed by means of lyophilization and the residue was dissolved in minimal amount of chloroform. The precipitated salts were discarded by filtration and the solvent applied to a silica gel column and eluted with petroleum ether—ethyl acetate (4.5:0.5) to furnish a pure, pale-yellow powder in 84% yield.

Synthesis of compound 15: Intermediate was dissolved in dry tetrahydrofuran and the solution was cooled and saturated with nitrogen dioxide gas. The reaction flask was capped and stirred at room temperature overnight. The flask was first cooled, carefully opened, and the solvent was evaporated to dryness. The residue was dissolved in minimal amount of ether and applied to a silica gel column and eluted with hexane to furnish pure compound 15 (11,17,21-trinitrato-16-DOXYL-betamethasone) in 90% yield.

### Example 13

### Synthesis of 11,17,21-trinitrato-16-DOXYL-beclomethasone (16)

The detailed synthesis of compound 16 was carried out as described in the following scheme:

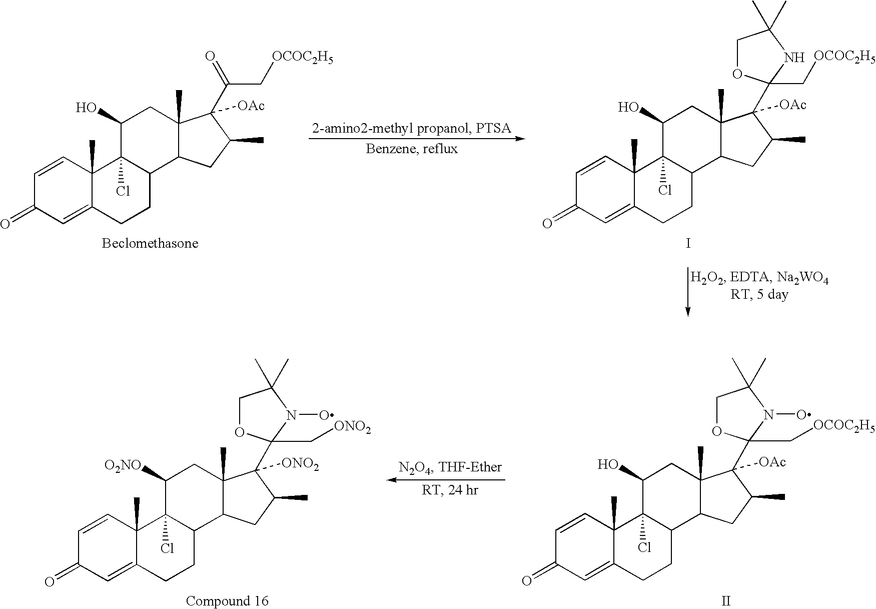

To a mixture of 3.92 g (10 mmol, 1 equivalent) of betamethasone and 2.22 g (25 mmol, 2.5 equivalents) of 2-amino-2-methylpropanol in benzene (100 ml), a catalytic amount of paratoluene sulfonic acid (PTSA) is added and the mixture is refluxed in Dein-Stark apparatus until starting material has disappeared (48-72 hr, TLC). After cooling, the benzene was evaporated to dryness and the solid residue is washed successively with distilled water and dried in a vacuum desiccator to give intermediate as a white powder in 92% yield, which was used for the next step without further purification.

Synthesis of intermediate II: To one equivalent of the intermediate dissolved in 50 ml of methanol, 10 equivalents of sodium tungstate (Na₂WO₄) dihydrate, and 10 equivalents of ethylene diamine tetraacetic acid (EDTA) disodium salt dissolved in 40 ml of distilled water, 10 ml of 30% hydrogen peroxide is added in three portions and the mixture is stirred in the dark at room temperature for one week (the reaction is usually complete within 4-6 days). Water is removed by means of lyophilization and the residue is dissolved in minimal amount of chloroform. The precipitated salts are discarded by filtration and the solvent applied to a silica gel column and eluted with petroleum ether—ethyl acetate (4.5:0.5) to furnish a pure, pale-yellow powder in 84% yield.

Synthesis of compound 16: Intermediate is dissolved in dry tetrahydrofuran and the solution is cooled and saturated with nitrogen dioxide gas. The reaction flask is capped and stirred at room temperature overnight. The flask is first cooled, carefully opened, and the solvent is evaporated to dryness. The residue is dissolved in minimal amount of ether and applied to a silica gel column and eluted with hexane to furnish pure compound 16 (11,17,21-trinitrato-16-DOXYL-beclomethasone) in 86% yield.

### Example 14

### Biological Activity

The potency and efficacy of the multifunctional steroid compounds are evaluated using a model of biological response for asthma as described below, where increased relaxation is an in vitro indication of increased efficacy.

Tissue Preparation

Male Hartley guinea pigs (500-600 g) are anesthetized by intraperitoneal injection of ketamine and xylazine (50 and 10 mg/kg, respectively). The heart and lungs are excised en bloc and tracheas are removed and placed in Krebs-Henseleit buffer composed of (mM): NaCl 118, KCl 5.4, NaH₂PO₄ 1.01, NaHCO₃ 25, MgSO₄ 0.69, CaCl₂ 2.32, glucose 11.1, pH 7.4. Tracheas are then dissected free from surrounding fat and connective tissue and cut into 1-2-mm thick rings. The tracheal rings are then placed in buffer and continuously gassed with 95% O₂ and 5% CO₂.

Relaxation Studies

Tracheal rings are suspended on stainless steel hooks in 10 ml of oxygenated (95% O₂, 5% CO₂) Krebs-Henseleit buffer at 37° C. and connected to transducer (Experimetria Model) for recording changes in isometric force. The tracheal rings are equilibrated for 60 min under a loads of 1 g and then primed twice by exposure to 100 μM methacholine. Tissues are contacted with methacholine, histamine, or leukotriene D₄ at concentrations determined to approximately generate 50% of maximal tone, after which cumulative relaxation-response curves are constructed. To construct these curves, the initial contraction is assigned a value of 100% and the bath concentration of the tested compound required to achieve 50% relaxation (i.e., IC₅₀) determined by linear interpolation. In some experiments, relaxation responses are determined in the presence of other drugs/agents or after rings had been pre-exposed to adenyl or guanylyl cyclase inhibitors for 30 min. Increased relaxation in tracheal rings exposed to multifunctional steroid compounds compared to controls indicates efficacy of the multifunctional steroid compounds as bronchorelaxants. This in vitro is predictive of in vivo efficacy for the treatment of respiratory disorders such as asthma

Cyclic Nucleotide Assays

Tracheal rings in Krebs-Heneseleit solution are exposed to a 5-100 μM of the tested compound or control for 30-90 seconds. Reaction is terminated by the addition of ice-cold 10% trichloroacetic acid and rapidly frozen in ethanol-saturated dry ice. In selected experiments, rings are pre-exposed to guanylyl cyclase inhibitors for 30 min. Tissue are then individually pulverized with a glass homogenizer and centrifuged at 8000×g for 5 min. The clear supernatant is extracted with water-saturated ether and assayed for cGMP by commercially available radioimmunoassay kits as described by the manufacturer. Stimulation of the production of cGMP is indicative of NO guanylyl cyclase pathway activity.

Abbreviations Used in Figures

“Bud ep 22S” is Budesonide epimer 22S,

“Tri” stands is Triamcinolone,

“Flu” is Fluticasone,

“Bec” is Beclomethasone,

“Mom” is Mometasone,

“Bet” is Betametasone,

“benz.” is benzene, and

“refl.” is reflux.

1. Use of a multifunctional steroid compound comprising
i) a steroid component,
ii) at least one SOD mimic component, and optionally
iii) at least one NO donor component in the preparation of a medicament.

2. Use of a multifunctional steroid compound according to claim 1, comprising
i) a steroid component,
ii) at least one SOD mimic component linked to said steroid component, and
iii) at least one NO donor component linked to said steroid component.

3. Use according to claim 1, wherein said steroid comprises cyclopenta[a]phenantrene, said SOD mimic component comprises an antioxidant reacting with superoxide, and said NO donor comprises a group capable of providing nitric oxide in a form selected from uncharged, free radical, and charged.

4. Use according to claim 1, wherein said SOD mimic component comprises a substituted N-oxide free radical.

5. Use according to claim 4, wherein the N-atom of said N-oxide is a member of 3 to 7 membered heterocyclic ring.

6. Use according to claim 2, wherein said NO donor component comprises a group selected from —ONO₂, —ONO, —SNO, and —NONOate.

7. Use of a multifunctional steroid compound according to claim 1 in the preparation of a medicament for treating or preventing a disorder selected from the group consisting of disorders associated with oxidative stress and free radical injury, disorders in which treatment with steroids or their analogs is indicated, and disorders in which treatment with a smooth muscle relaxant is indicated.

8. Use of a multifunctional steroid compound according to claim 1 in the preparation of a medicament for treating or preventing a disorder selected from the group consisting of respiratory, pulmonary, cardiovascular, inflammatory, and autoimmune disorders.

9. Use of a multifunctional steroid compound according to claim 1 in the preparation of a medicament for treating or preventing a disorder selected from the group consisting of asthma, chronic bronchitis, bronchiectasis, bronchospasms, emphysema, Chronic Obstructive Pulmonary Diseases (COPDs), bronchial hyperreactivity, respiratory distress syndrome or Chronic Obstructive Airway Disease (COADs), allergic conditions, arthritis, autoimmune hematologic disorders, systemic lupus erythematosus, systemic dermatomyositis, thrombocytopenia, psoriasis, contact dermatitis, atopic dermatitis, exfoliative dermatitis, acne, hirsutism, erythema nodosum, inflamed cysts, discoid lupus, bullous diseases, collagen vascular diseases, malignancies, neoplastic disease, trauma, shock, acute and chronic inflammatory conditions, sarcoidosis, Sweet's disease, graft-versus-host disease, multiple sclerosis, Alzheimer diseases, Parkinson's diseases, amyotrophic lateral sclerosis, convulsive disorders, AIDS-dementia, disorders related to learning, disorders related to olfaction, disorders related to nociception, cerebral edema, migraine, ophthalmic disorders, chronic adrenal insufficiency, congenital adrenal hyperplasia, gastrointestinal diseases, hepatic diseases, inflammatory bowel disease, Crohn's disease, ulcerative colitis, renal disease, gastric secretory and peristaltic functions, drug and disease-induced neuropathies and nephropathies, pathological uterine contractions, sinus tachycardia, ischaemic heart disease, angina pectoris, myocardial infarction, congestive heart failure, atherosclerosis, rheumatic disorders, hypertension, arrhythmia, hyperthyroidism, cellular defense impairment, hypercholestemia, Reaven's Syndrome, vasculitis, arteritis, endothelial dysfunction-induced diseases, diabetes mellitus, insulin-resistance and glucose intolerance in diabetes, ischemia-reperfusion tissue injury, chemotaxis and phagocytic impairment in immunological disorders, aging-mediated changes, cerebrovascular diseases, thyrotoxicosis, aggregation disorders, fertility conditions and reproductive disorders, menopause, ovarian dysfunction, testicular dysfunction, and penile erection.

10. Use of a multifunctional steroid compound according to claim 1, wherein said steroid compound has formula (4) 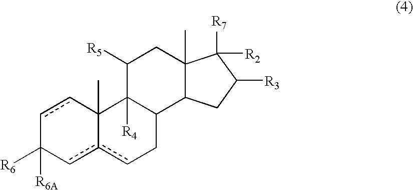
optical isomers thereof, salts thereof, and solvates thereof;
wherein [symbol] is a single or double bond, with the proviso that two double bonds are not adjacent;
R² is —H, —ONO, —ONO₂, —SNO, —OH, —CH₃, —NONOate, —OC(O)R⁸ wherein R⁸ is C₁-C₅ alkyl or 5- or 6-member heteroaryl, or R² and R⁷ together form a substituted N-oxide free radical;
R³ is —H, —OH, or —CH₃, or R and R³ together form a heterocyclic ring;
R⁴ is —H or halogen;
R⁵ is —H, ═O, —ONO, —ONO₂, —SNO, —NONOate or a substituted N-oxide free radical;
R⁶ is ═O, —ONO, —ONO₂, —SNO, —NONOate, and
R^{6A}, if present, is —H, or R⁶ and R^{6A} together form a substituted N-oxide free radical;
R⁷ is —H, —ONO, —ONO₂, —SNO, —NONOate, or a substituted N-oxide free radical wherein the nitrogen of the N-oxide group in the substituted N-oxide free radical is within a 5- or 6-member ring, which ring is optionally substituted by —OCOCH₂-PEG wherein said PEG may by optionally coupled to another steroid compound, and which ring is further optionally substituted by or one or more independently selected C₁-C₅ alkyl groups which may be further independently substituted by a group selected from an NO donor component, —SR¹¹, -halogen, and —OC(O)R¹³ wherein R¹¹ is C₁-C₅ alkyl and wherein R¹³ is C₁-C₅ alkyl or 5- or 6-member heteroaryl, or R² and R⁷ together form a substituted N-oxide free radical; and
wherein NO donor is a group comprising one of —ONO₂, —ONO, —SNO, and —NONOate, and wherein the nitrogen of the N-oxide group in the substituted N-oxide free radical is within a 5- or 6-member ring substituted by one or more independently selected C₁-C₅ alkyl groups which may be may be further independently substituted by an NO donor component.

11. Use according to claim 10, wherein said steroid compound has formula (5) 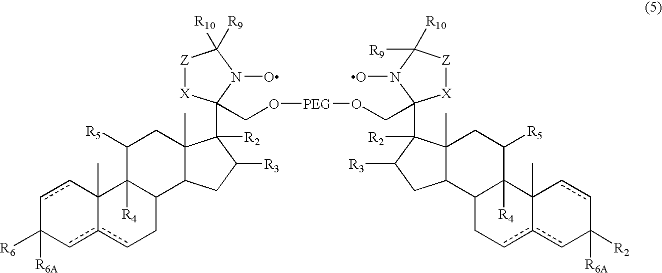
wherein the R², R³, R⁴, R⁵, R⁶, and R^{6A} are as defined in claim 10;
R⁹ and R¹⁰ are independently, linear or branched C₁-C₅ alkyl groups, or substituted linear or branched C₁-C₅ alkyl groups wherein the alkyl group is independently substituted by an NO donor or —OC(O)R⁴, wherein R¹⁴ is C₁-C₅ alkyl, or 5- or 6-member heteroaryl;
X is —CH₂—, —or —S—;
Z is —CH₂— or —CH₂—CH₂—;
and PEG is a polyethylene glycol of a molecular weight from about 100 to about 4000.

12. Use according to claim 10, wherein said steroid compound has a formula selected from Ia to Id (below) wherein
R² is —H, —ONO, —ONO₂, —SNO, —OH, —CH₃, —NONOate, or —OC(O)R⁸, wherein R⁸ is C₁-C₅ alkyl, or 5- or 6-member heteroaryl;
R³ is —H, —OH, or —CH₃, or R² and R³ together form a heterocyclic ring;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups;
R⁶ is ═O, —ONO, —ONO₂, —SNO, —NONOate, and
R^{6A}, if present, is —H, or R⁶ and R^{6A} together form a substituted N-oxide free radical, wherein the nitrogen of the N-oxide group in the substituted N-oxide free radical is within a 5- or 6-member ring substituted by one or more independently selected C₁-C₅ alkyl groups which may be may be further independently substituted by an NO donor component;
R⁷ is —H, —ONO, —ONO₂, —SNO, —NONOate, or a substituted N-oxide free radical, wherein the nitrogen of the N-oxide group in the substituted N-oxide free radical is within a 5- or 6-member ring optionally substituted by —OCOCH₂-PEG or one or more independently selected C₁-C₅ alkyl groups which may be further independently substituted by an NO donor component, —SR¹¹— halogen, or —OC(O)R¹³, wherein R¹¹ is C₁-C₅ alkyl, and wherein 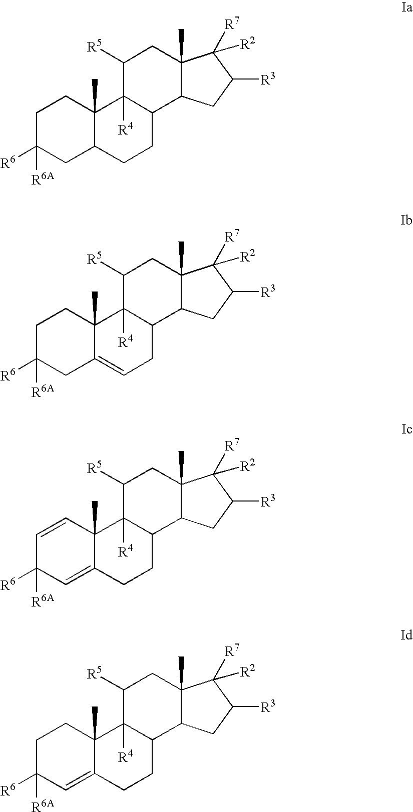
R¹³ is C₁-C₅ alkyl, or 5- or 6-member heteroaryl, or R² and R⁷ together form a substituted N-oxide free radical; and
NO donor is a group comprising one of —NO₂, —ONO, —SNO, and —NONOate;
wherein at least one of R², R⁵, R⁶, or R⁷ comprises an NO donor; and
wherein at least one of R⁵, R⁶, or R⁷ comprises a substituted N-oxide free radical.

13. Use according to claim 10, wherein said steroid compound has a formula selected from IIa to IId (below) wherein
R² is —H, —ONO, —ONO₂, —SNO, —OH, —CH₃, —NONOate, or —OC(O)R⁸, wherein R⁸ is C₁-C₅ alkyl, or 5- or 6-member heteroaryl;
R³ is —H, —OH, or —CH₃, or R² and R³ together form a heterocyclic ring;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups;
R⁷ is —H, —ONO, —ONO₂, —SNO, —NONOate, or a substituted N-oxide free radical, wherein the nitrogen of the N-oxide group in the substituted N-oxide free radical is within a 5- or 6-member ring substituted by —OCOCH₂-PEG or by one or more independently selected C₁-C₅ alkyl groups, wherein said alkyl group may be further independently substituted by an NO donor, —SR¹¹, -halogen, or —OC(O)R¹³, wherein R¹¹ is C₁-C₅ alkyl, and wherein R¹³ is C₁-C₅ alkyl or 5- or 6-member heteroaryl;
R⁹ and R¹⁰ are independently, linear or branched C₁-C₅ alkyl groups or substituted linear or branched C₁-C₅ alkyl groups wherein said alkyl group may be substituted by an NO donor or —OC(O)R¹⁴, wherein R¹⁴ is C₁-C₅ alkyl or 5- or 6-member heteroaryl; 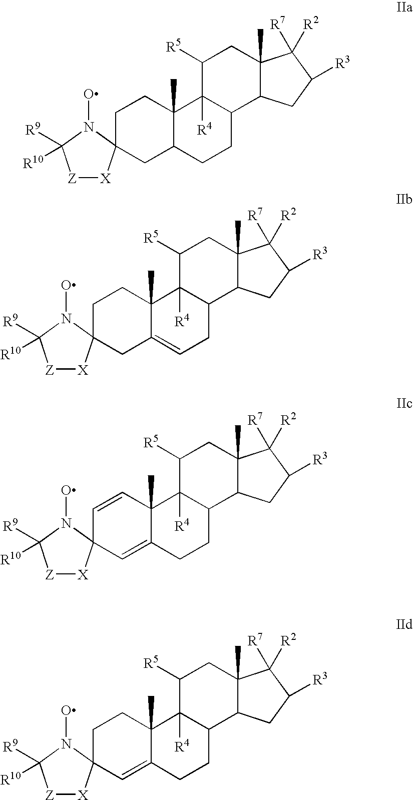
X is —CH₂—, —O— or —S—;
Z is —CH₂— or —CH₂—CH₂—;
NO donor is a group comprising one of —ONO₂, —ONO, —SNO, and —NONOate; and
wherein at least one of R², R⁵, R⁷, R⁹ or R¹⁰ comprises an NO donor.

14. Use according to claim 10, wherein said steroid compound has a formula selected from IIIa to IIId (below) wherein
R¹ is —H, —OH, —OCOCH₂-PEG, linear or branched C₁-C₅ alkyl, linear or branched C₁-C₅ alkyl substituted by an NO donor, —SR¹¹, -halogen, or —OC(O)R¹⁵, wherein R¹¹ is C₁-C₅ alkyl wherein R¹⁵ is C₁-C₅ alkyl;
R² is —H, —ONO, —ONO₂, —SNO, —OH, —CH₃, —NONOate, or —OC(O)R⁸, wherein R⁸ is C₁-C₅ alkyl, or 5- or 6-member heteroaryl;
R³ is —H, —OH, or —CH₃, or R and R³ together form a heterocyclic ring;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups;
R⁶ is ═O, —ONO, —ONO₂, —SNO, —NONOate, and R^{6A}, if present, is —H, or R⁶ and R^{6A} together form a substituted N-oxide free radical, wherein the nitrogen of the N-oxide group in the substituted N-oxide free radical is within a 5- or 6-member ring substituted by one or more independently selected C₁-C₅ alkyl groups, wherein said alkyl may be further substituted by an NO donor, or —OC(O)R¹², wherein R¹² is C₁-C₅ alkyl, or 5- or 6-member heteroaryl;
R⁹ and R¹⁰ are independently, linear or branched C₁-C₅ alkyl groups, or substituted linear or branched C₁-C₅ alkyl groups wherein said alkyl group is 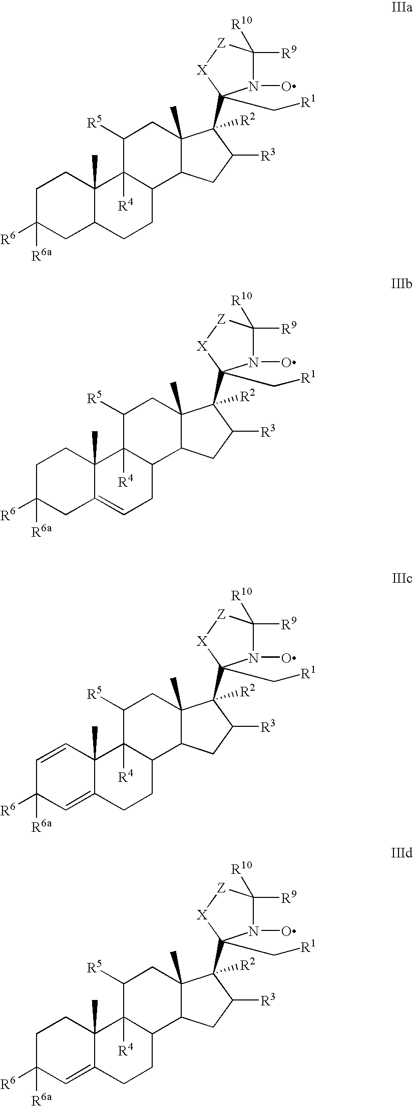
independently substituted by —ONO, —ONO₂, —SNO, —NONOate or —OC(O)R¹⁴, wherein R¹⁴ is C₁-C₅ alkyl;
X is —CH₂—, or —S—;
Z is —CH₂— or —CH₂—CH₂—;
wherein an NO donor is a group comprising one of —ONO₂, —ONO, —SNO, and —NONOate; and
wherein at least one of R¹, R², R⁵, R⁶, R⁹ or R¹⁰ comprises at least one NO donor.

15. Use according to claim 10, wherein said steroid compound has formula a formula selected from IVa to IVd (below) wherein
R¹ is —H, —OH, —OCOCH₂-PEG; linear or branched C₁-C₅ alkyl; linear or branched C₁-C₅ alkyl substituted by an NO donor, —SR¹¹, -halogen, or —OC(O)R⁵, wherein R¹¹ is C₁-C₅ alkyl, and wherein R¹⁵ is C₁-C₅ alkyl;
R² is —H, —ONO, —ONO₂, —SNO, —OH, —CH₃, —NONOate, or —OC(O)R⁸, wherein R⁸ is C₁-C₅ alkyl, or 5- or 6-member heteroaryl;
R³ is —H, H, or —CH₃, or R² and R³ together form a heterocyclic ring;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups; R⁹ and R¹⁰ are independently, linear or branched C₁-C₅ alkyl groups, or substituted linear or branched C₁-C₅ alkyl groups wherein the said group is independently substituted by an NO donor or —OC(O)R¹⁴,
wherein R¹⁴ is C₁-C₅ alkyl;
X is —CH₂—, —O— or —S—;
Z is —CH₂— or —CH₂—CH₂—; 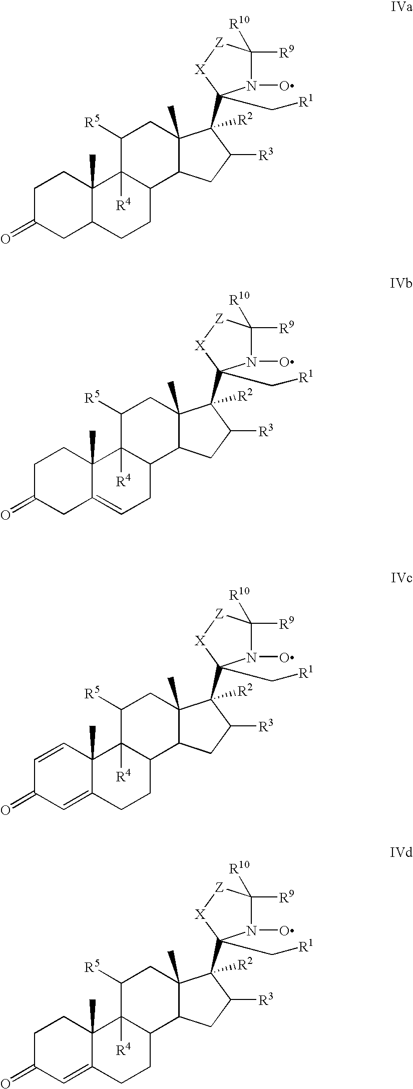
wherein an NO donor is a group comprising one of —ONO₂, —ONO, —SNO, and —NONOate; and
wherein at least one of R¹, R², R⁵, R⁹ or R¹⁰ comprises at least one NO donor.

16. Use according to claim 10, wherein said steroid compound has a formula selected from Va to Vd (below) wherein
R³ is —H, —OH, or —CH₃;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups;
R⁶ is ═O, —ONO, —ONO₂, —SNO, —NONOate,
and R^{6A}, if present, is —H, or R⁶ and R^{6A} together form a substituted N-oxide free radical, wherein the nitrogen of the N-oxide group in the substituted N-oxide free radical is within a 5- or 6-member ring substituted by one or more independently selected C₁-C₅ alkyl groups wherein said alkyl groups may be further substituted by an NO donor, or —OC(O)R¹², wherein R¹² is C₁-C₅ alkyl, or 5- or 6-member heteroaryl;
R⁹ and R¹⁰ are independently, linear or branched C₁-C₅ alkyl groups, or substituted linear or branched C₁-C₅ alkyl groups wherein the alkyl group is independently substituted by an NO donor or —OC(O)R¹⁴, wherein R¹⁴ is C₁-C₅ alkyl, or 5- or 6-member heteroaryl;
X is —CH₂—, —O— or —S—;
Z is —CH₂— or —CH₂—CH₂—;
wherein an NO donor is a group comprising one of —ONO₂, —ONO, —SNO, and —NONOate; and
wherein at least one of R⁵, R⁶, R⁹ or R¹⁰ comprises an NO donor.

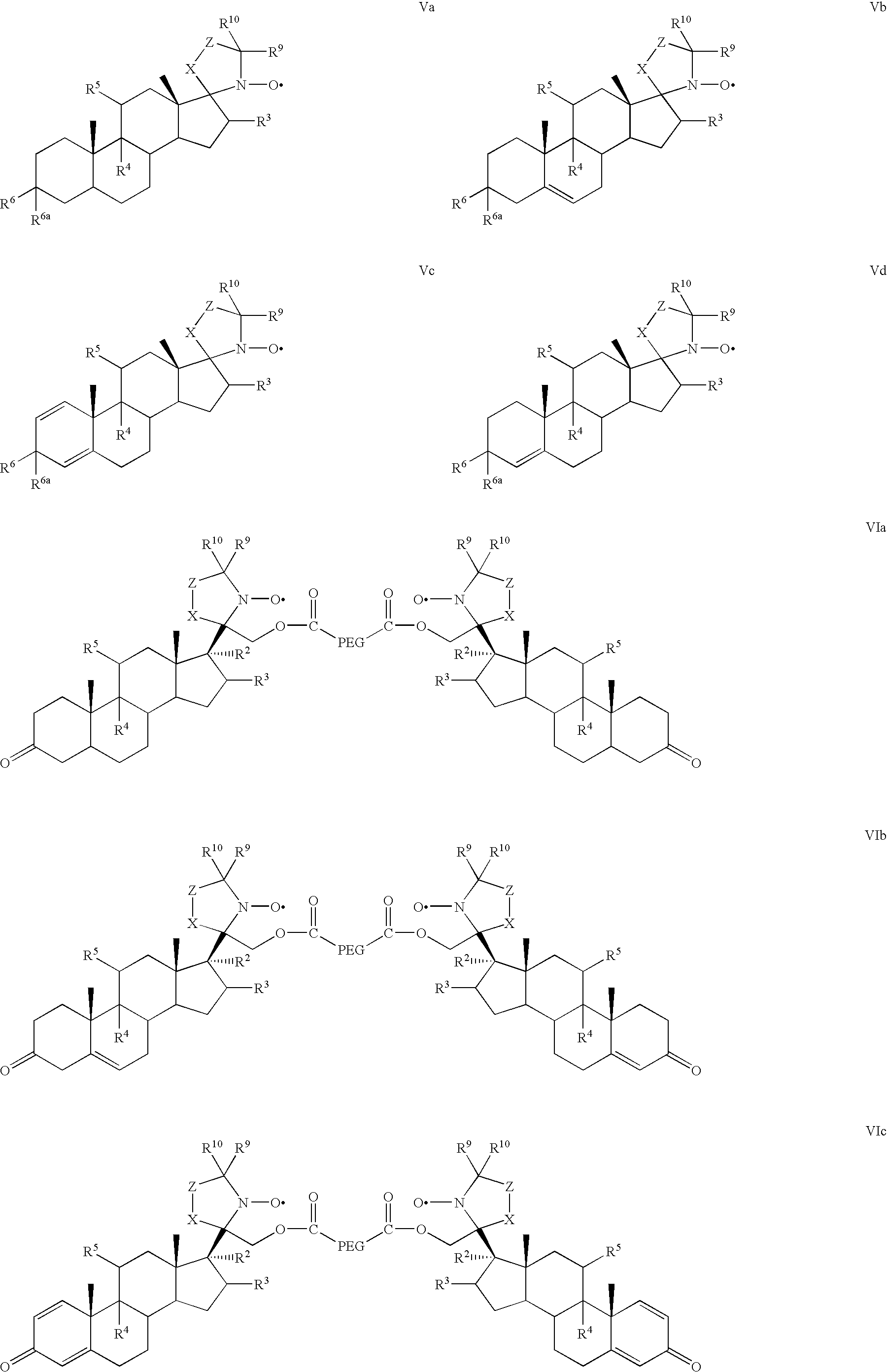

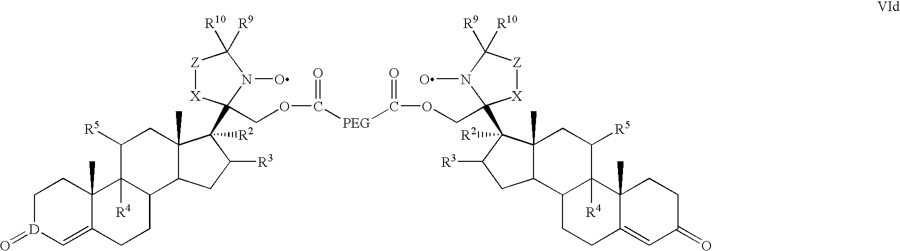

17. Use according to claim 11, wherein said steroid compound has a formula selected from VIa to VId (above) wherein
R² is —H, —ONO, —ONO₂, —SNO, —OH, —CH₃, —NONOate, or —OC(O)R⁸, wherein R⁸ is C₁-C₅ alkyl, or 5- or 6-member heteroaryl;
R³ is —H, —OH, or —CH₃, or R² and R³ together form a heterocyclic ring;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups;
R⁹ and R¹⁰ are independently, linear or branched C₁-C₅ alkyl groups, or substituted linear or branched C₁-C₅ alkyl groups wherein the alkyl group is independently substituted by an NO donor or —OC(O)R¹⁴, wherein R¹⁴ is C₁-C₅ alkyl, or 5- or 6-member heteroaryl;
X is —CH₂—, —O— or —S—;
Z is —CH₂— or —CH₂—CH₂—;
wherein an NO donor is a group comprising one of —ONO₂, —ONO, —SNO, and —NONOate; and
wherein at least one of R², R⁵, R⁹ or R¹⁰ comprises at least one NO donor.

18. Use according to claim 10 wherein R³ is —H, —OH, or —CH₃; R⁴ is —H, F, or Cl; and R⁵ is —H, ═O, or —ONO₂.

19. Use according to claim 11 wherein X is —CH₂— or —O—, Z is —CH₂—, and R⁹ and R¹⁰ are independently methyl or ethyl.

20. Use according to claim 10 wherein R² is —H or —ONO₂.

21. Use according to claim 10 wherein R⁶ is ═O, —ONO₂, and R^{6A}, if present, is —H, or R⁶ and R^{6A} together form a substituted N-oxide free radical selected from substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical and substituted thiazinyloxy N-oxide free radical.

22. Use according to claim 10 wherein R⁷ is —ONO₂ or a substituted N-oxide free radical selected from substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical and substituted thiazinyloxy N-oxide free radical.

23. Use according to claim 10 wherein said N-oxide free radical is selected from the substituted 5- or 6-member rings of general formulae 3a and 3b 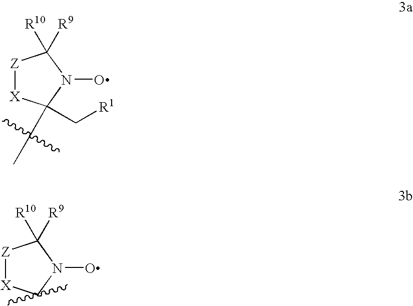
wherein X is —CH₂—, —O— or —S—;
Z is —CH₂— or —CH₂—CH₂—;
R¹ is —H, —OH, —OCOCH₂-PEG, linear or branched C₁-C₅ alkyl, linear or branched C₁-C₅ alkyl substituted by —ONO, —ONO₂, —SNO, or —NONOate or —OC(O)R¹⁵, wherein R¹⁵ is C₁-C₅ alkyl, or 5- or 6-member heteroaryl; and R⁹ and R¹⁰ are independently, linear or branched C₁-C₅ alkyl groups, or substituted linear or branched C₁-C₅ alkyl groups, wherein said alkyl group may be is independently substituted by —ONO, —ONO₂, —SNO, —NONOate or —OC(O)R¹⁴, wherein R¹⁴ is C₁-C₅ alkyl, or 5- or 6-member heteroaryl.

24. A multifunctional steroid compound comprising
i) a steroid component,
ii) at least one SOD mimic component, and
iii) at least one NO donor component, for use as a medicament.

25. A multifunctional steroid compound according to claim 24, wherein said steroid component is selected from corticosteroids, estrogens, progesterones, androgens, analogs thereof, and derivatives thereof.

26. A method of treating or preventing a disorder selected from the group consisting of asthma, chronic bronchitis, bronchiectasis, bronchospasms, emphysema, Chronic Obstructive Pulmonary Diseases (COPDs), bronchial hyperreactivity, respiratory distress syndrome or Chronic Obstructive Airway Disease (COADs), allergic conditions, arthritis, autoimmune hematologic disorders, systemic lupus erythematosus, systemic dermatomyositis, thrombocytopenia, psoriasis, contact dermatitis, atopic dermatitis, exfoliative dermatitis, acne, hirsutism, erythema nodosum, inflamed cysts, discoid lupus, bullous diseases, collagen vascular diseases, malignancies, neoplastic disease, trauma, shock, acute and chronic inflammatory conditions, sarcoidosis, Sweet's disease, graft-versus-host disease, multiple sclerosis, Alzheimer diseases, Parkinson's diseases, amyotrophic lateral sclerosis, convulsive disorders, AIDS-dementia, disorders related to learning, disorders related to olfaction, disorders related to nociception, cerebral edema, migraine, ophthalmic disorders, chronic adrenal insufficiency, congenital adrenal hyperplasia, gastrointestinal diseases, hepatic diseases, inflammatory bowel disease, Crohn's disease, ulcerative colitis, renal disease, gastric secretory and peristaltic functions, drug and disease-induced neuropathies and nephropathies, pathological uterine contractions, sinus tachycardia, ischaemic heart disease, angina pectoris, myocardial infarction, congestive heart failure, atherosclerosis, rheumatic disorders, hypertension, arrhythmia, hyperthyroidism, cellular defense impairment, hypercholestemia, Reaven's Syndrome, vasculitis, arteritis, endothelial dysfunction-induced diseases, diabetes mellitus, insulin-resistance and glucose intolerance in diabetes, ischemia-reperfusion tissue injury, chemotaxis and phagocytic impairment in immunological disorders, aging-mediated changes, cerebrovascular diseases, thyrotoxicosis, aggregation disorders, fertility conditions and reproductive disorders, menopause, ovarian dysfunction, testicular dysfunction, and penile erection, in a mammal in need thereof comprising administering to said mammal an effective amount of a a multifunctional steroid compound comprising
i) a steroid component,
ii) at least one SOD mimic component, and optionally
iii) at least one NO donor component.

27. A method according to claim 26, wherein said administration or treatment is selected from the group consisting of topical, oral, and parenteral.

28. A method according to claim 26, wherein said administration or treatment is selected from the group consisting of suppository, by way of injection, and by way of infusion.

29. A method according to claim 26, wherein said multifunctional steroid compound is administered by a route selected from intramuscular, intraperitoneal, intravenous, ICV, intracistemal injection or infusion, subcutaneous injection, implant, inhalation spray, nasal, vaginal, rectal, sublingual, and urethral.

30. A method according to claim 26, wherein said mammal is human.

31. A multifunctional steroid compound of formula (4) 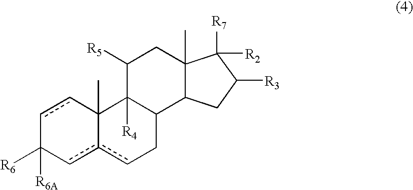
optical isomers thereof, salts thereof, and solvates thereof;
wherein [symbol] is a single or double bond, with the proviso that two double bonds are not adjacent;
R² is NO donor, —H, —OH, —CH₃, —OC(O)R⁸ wherein R⁸ is C₁-C₅ alkyl or 5- or 6-member heteroaryl, or R² and R⁷ together form a substituted N-oxide free radical;
R³ is —H, —OH, or —CH₃, or R² and R³ together form a heterocyclic ring;
R⁴ is —H or halogen;
R⁵ is —H, ═O, NO donor or a substituted N-oxide free radical;
R⁶ is ═O or NO donor, and
R^{6A}, if present, is —H, or R⁶ and R^{6A} together form a substituted N-oxide free radical;
R⁷ is —H, NO donor, or a substituted N-oxide free radical wherein the nitrogen of the N-oxide group in the substituted N-oxide free radical is within a 5- or 6-member ring, which ring is optionally substituted by —OCOCH₂-PEG wherein said PEG may by optionally coupled to another steroid compound, and which ring is further optionally substituted by or one or more independently selected C₁-C₅ alkyl groups which may be further independently substituted by a group selected from an NO donor component, —SR¹¹, -halogen, and —OC(O)R¹³ wherein R¹¹ is C₁-C₅ alkyl and wherein R¹³ is C₁-C₅ alkyl or 5- or 6-member heteroaryl, or R² and R⁷ together form a substituted N-oxide free radical; and
wherein the nitrogen of the N-oxide group in the substituted N-oxide free radical is within a 5- or 6-member ring substituted by one or more independently selected C₁-C₅ alkyl groups which may be further independently substituted by an NO donor component; and wherein said NO donor is a group comprising one of —ONO₂, —ONO, —SNO, and —NONOate; and with the proviso that said compound contains at least one N-oxide free radical and at least one NO donor.

32. A multifunctional steroid compound according claim 31, wherein said PEG links two identical structures selected from the group consisting of Ia to Id, IIa to IId, IIIa to IIId, and IVa to IVd.

33. A compound according to claim 32, having formula (5) 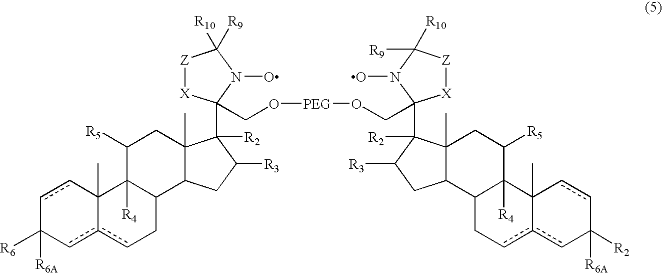
wherein the R², R³, R⁴, R⁵, R⁶, and R^{6A} are as defined in claim 31;
R⁹ and R¹⁰ are independently, linear or branched C₁-C₅ alkyl groups, or substituted linear or branched C₁-C₅ alkyl groups wherein the alkyl group is independently substituted by an NO donor or —OC(O)R¹⁴, wherein R¹⁴ is C₁-C₅ alkyl, or 5- or 6-member heteroaryl;
X is —CH₂—, —O— or —S—;
Z is —CH₂— or —CH₂—CH₂—;
and PEG is a polyethylene glycol of a molecular weight from about 100 to about 4000.

34. A compound according to claim 31, having a formula selected from Ia to Id (page 106) wherein
R² is —H, —ONO, —ONO₂, —SNO, —OH, —CH₃, —NONOate, or —OC(O)R⁸, wherein R⁸ is C₁-C₅ alkyl, or 5- or 6-member heteroaryl;
R³ is —H, —OH, or —CH₃, or R² and R³ together form a heterocyclic ring;
R⁴ is H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups;
R⁶ is ═O, —ONO, —ONO₂, —SNO, —NONOate, and
R^{6A}, if present, is —H, or R⁶ and R^{6A} together form a substituted N-oxide free radical, wherein the nitrogen of the N-oxide group in the substituted N-oxide
free radical is within a 5- or 6-member ring substituted by one or more independently selected C₁-C₅ alkyl groups which may be may be further independently substituted by an NO donor component;
R⁷ is —H, —ONO, —ONO₂, —SNO, —NONOate, or a substituted N-oxide free radical, wherein the nitrogen of the N-oxide group in the substituted N-oxide free radical is within a 5- or 6-member ring optionally substituted by —OCOCH₂-PEG or one or more independently selected C₁-C₅ alkyl groups which may be further independently substituted by an NO donor component, —SR¹¹— halogen, or —OC(O)R¹³, wherein R¹¹ is C₁-C₅ alkyl, and wherein R¹³ is C₁-C₅ alkyl, or 5- or 6-member heteroaryl, or R² and R⁷ together form a substituted N-oxide free radical; and
NO donor is a group comprising one of —ONO₂, —ONO, —SNO, and —NONOate;
wherein at least one of R², R⁵, R⁶, or R⁷ comprises an NO donor; and
wherein at least one of R⁵, R⁶, or R⁷ comprises a substituted N-oxide free radical.

35. A compound according to claim 31, having a formula selected from IIa to IId (page 108) wherein
R² is —H, —ONO, —ONO₂, —SNO, —OH, —CH₃, —NONOate, or —OC(O)R⁸, wherein R⁸ is C₁-C₅ alkyl, or 5- or 6-member heteroaryl;
R³ is —H, —OH, or —CH₃, or R² and R³ together form a heterocyclic ring;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups;
R⁷ is —H, —ONO, —ONO₂, —SNO, —NONOate, or a substituted N-oxide free radical, wherein the nitrogen of the N-oxide group in the substituted N-oxide free radical is within a 5- or 6-member ring substituted by
—OCOCH₂-PEG or by one or more independently selected C₁-C₅ alkyl groups, wherein said alkyl group may be further independently substituted by an NO donor, —SR¹¹—, -halogen, or —OC(O)R¹³, wherein R¹³ is C₁-C₅ alkyl, and wherein R¹³ is C₁-C₅ alkyl or 5- or 6-member heteroaryl;
R⁹ and R¹⁰ are independently, linear or branched C₁-C₅ alkyl groups or substituted linear or branched C₁-C₅ alkyl groups wherein said alkyl group may be substituted by an NO donor or —OC(O)R¹⁴, wherein R¹⁴ is C₁-C₅ alkyl or 5- or 6-member heteroaryl;
X is —CH₂—, —O— or —S—;
Z is —CH₂— or —CH₂—CH₂—;
NO donor is a group comprising one of —ONO₂, —ONO, —SNO, and —NONOate; and
wherein at least one of R², R⁵, R⁷, R⁹ or R¹⁰ comprises an NO donor.

36. A compound according to claim 31, having a formula selected from IIIa to IIId (page 110) wherein
R¹ is —H, —OH, —OCOCH₂-PEG, linear or branched C₁-C₅ alkyl, linear or branched C₁-C₂ alkyl substituted by an NO donor, —SR¹¹, -halogen, or —OC(O)R¹⁵, wherein R¹¹ is C₁-C₅ alkyl, wherein R¹⁵ is C₁-C₅ alkyl;
R² is —H, —ONO, —ONO₂, —SNO, —OH, —CH₃, —NONOate, or —OC(O)R⁸, wherein R⁸ is C₁-C₅ alkyl, or 5- or 6-member heteroaryl;
R³ is —H, —OH, or —CH₃, or R² and R³ together form a heterocyclic ring;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups;
R⁶ is ═O, —ONO, —ONO₂, —SNO, —NONOate, and R^{6A} if present is —H, or R⁶ and R^{6A} together form a substituted N-oxide free radical, wherein the nitrogen of the N-oxide group in the substituted N-oxide free radical is within a 5- or 6-member ring substituted by one or more independently selected C₁-C₅ alkyl groups, wherein said alkyl may be further substituted by an NO donor, or —OC(O)R¹², wherein R¹² is C₁-C₅ alkyl, or 5- or 6-member heteroaryl;
R⁹ and R¹⁰ are independently, linear or branched C₁-C₅ alkyl groups, or substituted linear or branched C₁-C₅ alkyl groups wherein said alkyl group is independently substituted by —ONO, —ONO₂, —SNO, —NONOate or —OC(O)R¹⁴, wherein R⁴ is C₁-C₅ alkyl
X is —CH₂—, —O— or —S—;
Z is —CH₂— or —CH₂—CH₂—;
wherein an NO donor is a group comprising one of —ONO₂, —ONO, —SNO, and —NONOate; and
wherein at least one of R¹, R², R⁵, R⁶, R⁹ or R¹⁰ comprises at least one NO donor.

37. A compound according to claim 31, having a formula selected from IVa to IVd (page 112) wherein
R¹ is —H, —OH, —OCOCH₂-PEG; linear or branched C₁-C₅ alkyl; linear or branched C₁-C₅ alkyl substituted by an NO donor, —SR¹¹, -halogen, or —OC(O)R¹⁵, wherein R¹⁵ is C₁-C₅ alkyl and wherein R¹⁵ is C₁-C₅ alkyl;
R² is —H, —ONO, —ONO₂, —SNO, —OH, —CH₃, —NONOate, or —OC(O)R⁸, wherein R⁸ is C₁-C₅ alkyl, or 5- or 6-member heteroaryl;
R³ is —H, —OH, or —CH₃, or R and R³ together form a heterocyclic ring;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups; R⁹ and R¹⁰ are independently, linear or branched C₁-C₅ alkyl groups, or substituted linear or branched C₁-C₅ alkyl groups wherein the said group is independently substituted by an NO donor or —OC(O)R¹⁴,
wherein R¹⁴ is C₁-C₅ alkyl;
X is —CH₂—, —O— or —S—;
Z is —CH₂— or —CH₂—CH₂—;
wherein an NO donor is a group comprising one of —ONO₂, —ONO, —SNO, and —NONOate; and
wherein at least one of R¹, R², R⁵, R⁶ or R¹⁰ comprises at least one NO donor.

38. A compound according to claim 31, having a formula selected from Va to Vd (page 114) wherein
R³ is —H, H, or —CH₃;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups;
R⁶ is ═O, —ONO, —ONO₂, —SNO, —NONOate,
and R^{6A}, if present, is —H, or R⁶ and R^{6A} together form a substituted N-oxide free radical, wherein the nitrogen of the N-oxide group in the substituted N-oxide free radical is within a 5- or 6-member ring substituted by one or more independently selected C₁-C₅ alkyl groups wherein said alkyl groups may be further substituted by an NO donor, or —OC(O)R¹², wherein R¹² is C₁-C₅ alkyl, or 5- or 6-member heteroaryl;
R⁹ and R¹⁰ are independently, linear or branched C₁-C₅ alkyl groups, or substituted linear or branched C₁-C₅ alkyl groups wherein the alkyl group is independently substituted by an NO donor or —OC(O)R¹⁴, wherein R¹⁴ is C₁-C₅ alkyl, or 5- or 6-member heteroaryl;
X is —CH₂—, —O— or —S—;
Z is —CH₂— or —CH₂—CH₂—;
wherein an NO donor is a group comprising one of —ONO₂, —ONO, —SNO, and —NONOate; and
wherein at least one of R⁵, R⁶, R⁹ or R¹⁰ comprises an NO donor.

39. A compound according to claim 32, having a formula selected from VIa to VId (page 115) wherein
R² is —H, —ONO, —ONO₂, —SNO, —OH, —CH₃, —NONOate, or —OC(O)R⁸, wherein R⁸ is C₁-C₅ alkyl, or 5- or 6-member heteroaryl;
R³ is —H, —OH, or —CH₃, or R² and R³ together form a heterocyclic ring;
R⁴ is —H or halogen;
R⁵ is —H, ═O, —ONO, —ONO₂, —SNO, —NONOate or a substituted N-oxide free radical, wherein the nitrogen of the N-oxide group in the substituted N-oxide free radical is within a 5- or 6-member ring substituted by one or more independently selected C₁-C₅ alkyl groups;
R⁹ and R¹⁰ are independently, linear or branched C₁-C₅ alkyl groups, or substituted linear or branched C₁-C₅ alkyl groups wherein the alkyl group is independently substituted by an NO donor or —OC(O)R⁴, wherein R⁴ is C₁-C₅ alkyl, or 5- or 6-member heteroaryl;
X is —CH₂—, —O— or —S—;
Z is —CH₂— or —CH₂—CH₂—;
wherein an NO donor is a group comprising one of —ONO₂, —ONO, —SNO, and —NONOate; and
wherein at least one of R², R⁵, R⁹ or R¹⁰ comprises at least one NO donor.

40. A compound according to claim 31 wherein R³ is —H, —OH, or —CH₃; R⁴ is —H, F, or Cl; and R⁵ is —H, ═O, or —ONO₂.

41. A compound according to claim 33 wherein X is —CH₂— or —O—, Z is —CH₂—, and R⁹ and R¹⁰ are independently methyl or ethyl.

42. A compound according to claim 31 wherein R² is —H or —ONO₂.

43. A compound according to claim 31 wherein R⁶ is ═O, —ONO₂, and R^{6A}, if present, is —H, or R⁶ and R^{6A} together form a substituted N-oxide free radical selected from substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical and substituted thiazinyloxy N-oxide free radical.

44. A compound according to claim 31 wherein R⁷ is —ONO₂ or a substituted N-oxide free radical selected from substituted pyrrolidinyloxy N-oxide free radical, substituted piperidinyloxy N-oxide free radical, substituted oxazolidinyloxy N-oxide free radical, substituted oxazinyloxy N-oxide free radical, substituted thiazolidinyloxy N-oxide free radical and substituted thiazinyloxy N-oxide free radical.

45. A compound according to claim 31 wherein said N-oxide free radical is selected from the substituted 5- or 6-member rings of general formulae 3a and 3b 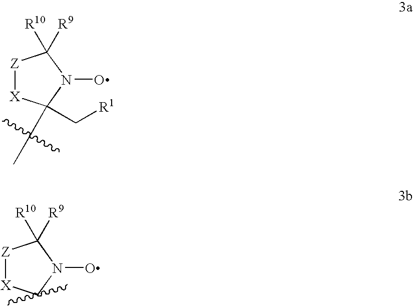
wherein X is —CH₂—, —O— or —S—;
Z is —CH₂— or —CH₂—CH₂—;
R¹ is —H, —OH, —OCOCH₂-PEG, linear or branched C₁-C₅ alkyl, linear or branched C₁-C₅ alkyl substituted by —ONO, —ONO₂, —SNO, or —NONOate or —OC(O)R¹⁵, wherein R¹⁵ is C₁-C₅ alkyl, or 5- or 6-member heteroaryl; and R⁹ and R¹⁰ are independently, linear or branched C₁-C₅ alkyl groups, or substituted linear or branched C₁-C₅ alkyl groups, wherein said alkyl group may be is independently substituted by —ONO, —oNO₂, —SNO, —NONOate or —OC(O)R¹⁴, wherein R¹⁴ is C₁-C₅ alkyl, or 5- or 6-member heteroaryl.

46. A pharmaceutical composition comprising a compound according to claim 31.

47. A pharmaceutical composition according to claim 46, further comprising a component selected from carrier, binding agent, stabilizer, adjuvant, diluent, excipient, surfactant, odorant, and second pharmaceutically active agent.

48. A pharmaceutical composition according to claim 46, for use as a medicament in treating and preventing a disorder selected from the group consisting of asthma, chronic bronchitis, bronchiectasis, bronchospasms, emphysema, pneumonia, Chronic Obstructive Pulmonary Diseases (COPDs), bronchial hyperreactivity, respiratory distress syndrome or Chronic Obstructive Airway Disease (COADs), allergic conditions, arthritis, autoimmune hematologic disorders, systemic lupus erythematosus, systemic dermatomyositis, thrombocytopenia, psoriasis, contact dermatitis, atopic dermatitis, exfoliative dermatitis, acne, hirsutism, erythema nodosum, inflamed cysts, discoid lupus, bullous diseases, collagen vascular diseases, malignancies, neoplastic disease, trauma, shock, acute and chronic inflammatory conditions, sarcoidosis, Sweet's disease, graft-versus-host disease, multiple sclerosis, Alzheimer diseases, Parkinson's diseases, amyotrophic lateral sclerosis, convulsive disorders, AIDS-dementia, disorders related to learning, disorders related to olfaction, disorders related to nociception, cerebral edema, migraine, ophthalmic disorders, chronic adrenal insufficiency, congenital adrenal hyperplasia, gastrointestinal diseases, hepatic diseases, inflammatory bowel disease, Crohn's disease, ulcerative colitis, renal disease, gastric secretory and peristaltic functions, drug and disease-induced neuropathies and nephropathies, pathological uterine contractions, sinus tachycardia, ischaemic heart disease, angina pectoris, myocardial infarction, congestive heart failure, atherosclerosis, rheumatic disorders, hypertension, arrhythmia, hyperthyroidism, cellular defense impairment, hypercholestemia, Reaven's Syndrome, vasculitis, arteritis, endothelial dysfunction-induced diseases, diabetes mellitus, insulin-resistance and glucose intolerance in diabetes, ischemia-reperfusion tissue injury, chemotaxis and phagocytic impairment in immunological disorders, aging-mediated changes, cerebrovascular diseases, thyrotoxicosis, aggregation disorders, fertility conditions and reproductive disorders, menopause, ovarian dysfunction, testicular dysfunction, and penile erection.

49. A kit for administration of a multifunctional steroid compound comprising i) a dosage amount of at least one multifunctional steroid compound that comprises a steroid component, at least one SOD mimic component, and optionally at least one NO donor component;
ii) instructions for use; and
iii) optionally means for the delivery of said compound.

50. A kit according to claim 49 comprising one of items selected from inhaler, spray dispenser, syringe, or suppositories.
